(19) United States
(12) Patent Application Publication
(10) Pub. No.: US 2009/0176837 A1
(43) Pub. Date: Jul. 9, 2009

(54) COMPOUNDS WITH ACTIVITY AT RETINOIC ACID RECEPTORS

(75) Inventors: Roger Olsson, Bunkeflostrand (SE); Fabrice Piu, San Diego, CA (US); Birgitte Lund, Bagsvaerd (DK)

Correspondence Address: KNOBBE MARTENS OLSON & BEAR LLP, 2040 MAIN STREET, FOURTEENTH FLOOR, IRVINE, CA, 92614, US

(73) Assignee: SONY CORPORATION, TOKYO (JP)

(21) Appl. No.: 11/995,528

(22) PCT Filed: Jul. 12, 2006
(86) PCT No.: PCT/US06/27448

§ 371 Date: Aug. 28, 2008

(51) Int. Cl. A61K 31/192; A61K 31/443; A61K 31/44; A61K 31/426; A61K 31/4196; A61K 31/4164; A61K 31/34; C07D 405/12; C07D 213/72; C07D 213/55; C07D 277/34; C07D 249/08; C07D 233/18; C07D 307/54; C07C 63/331; A61P 29/00; A61P 25/00; A61P 35/00

(52) U.S. Cl. 514/336; 514/277; 514/352; 514/369; 514/383; 514/400; 514/461; 514/568; 546/283.4; 546/312; 546/342; 548/187; 548/266.8; 548/334.1; 549/501; 562/492

(57) ABSTRACT

Disclosed herein are novel compounds with activity at RARβ 2 receptors. Further disclosed are the use of such compounds for treatment of or to alleviate symptoms of cancer, neurological disorders such as memory deficits and schizophrenia, neurodegenerative disorders such as Parkinson's and Alzheimer's diseases, inflammatory disorders such as psoriasis and rheumatoid arthritis, eye disorders and depression.

### RELATED APPLICATIONS

This application claims the benefit of U.S. Provisional Applications Nos. 60/698,622, filed Jul. 12, 2005, and 60/775,523, filed Feb. 21, 2006, both of which are incorporated herein by reference in their entirety including all examples, figures, and appendices therein.

### FIELD OF THE INVENTION

Aspects of the invention described below generally relate to compounds affecting a response at receptors of the nuclear receptor family, and more specifically the retinoic acid receptor subtype β isoform 2 (RARβ 2). Additionally, disclosed are the use of such compounds to alleviate symptoms of cancer, schizophrenia, depression, memory deficits, Parkinson's and Alzheimer's diseases, inflammatory disorders such as psoriasis, and rheumatoid arthritis and to improve the development and maintenance of the ocular surface in eye disorders/conditions.

### BACKGROUND OF THE INVENTION

Retinoids are small, lipophilic molecules that derive from the metabolism of vitamin A, a dietary vitamin. Natural and synthetic retinoid derivatives exert pleiotropic effects on cellular growth, differentiation, apoptosis, homeostasis and embryogenesis. A number of non-selective retinoids are currently marketed or undergoing clinical trials for use in dermatology and oncology. For instance, Tretinoin (all-trans-retinoic acid), Isotretinoin (13-cis retinoic acid) and Etretinate (a synthetic retinoic acid analog) are being used successfully in the treatment of acne, psoriasis, photoaging and squamous cell carcinoma. However, acute and chronic toxic side effects (skeletal abnormality, skin toxicity, triglyceride elevation, teratogenesis) are commonly observed which can lead to the discontinuation of the treatment.

The biological effects of retinoids are mediated by two classes of nuclear hormone receptors, the retinoic acid receptors (RARs) and the retinoid X receptors (RXRs). RARs and RXRs are ligand-dependent transcription factors belonging to the steroid nuclear receptor superfamily. Like the majority of nuclear receptors, the retinoid receptors display a modular structure: a N-terminus ligand-independent activation domain (AF-1), a DNA-binding domain (DBD) adjacent to the ligand-dependent domain (LBD) and the ligand-dependent activation domain (AF-2) contiguous to the LBD and located at the C-terminus end. Three receptors subtypes have been reported for each of the RARs and RXRs, classified as α, β, and γ-All six subtypes have reportedly distinct expression patterns in the developing embryo and in the adult, thus are believed to exhibit specific and non-overlapping functions.

Moreover, several isoforms have been described for each RAR and RXR subtypes. In particular, RARβ consists of four known isoforms generated from the use of two promoters P1 (RARβ 1 and RARβ 3) and P2 (RARβ 2 and RARβ 4). The isoforms only differ in the nature of their AF-1 transcriptional activation domains located at the very N-terminus. In particular, RARβ 1 and RARβ 3 have very similar AF-1 domains, the only difference being the presence of an additional 27 amino acids insert in RARβ 3. RARβ 2, on the other hand, has a unique AF-1 domain, while RARβ 4 lacks such a domain as well as a portion of its DNA binding domain (DBD). Data from the literature supports the notion that because of its truncated DNA domain, RARβ 4 could act as a dominant negative mutant. Interestingly, the isoforms have distinct spatial and temporal distribution. For example, in the mouse, RARβ 1 and RARβ 3 display a relatively restricted pattern, highly present the brain, and in limited amounts in the lung and skin. RARβ 2 is more broadly expressed, in particular in the brain and heart, and at much lower levels in the liver, kidney and skeletal muscle. In humans, only the RARβ 1, 2 and 4 isoforms are expressed.

RARβ 2 modulating compounds may be used to treat cancer. A growing body of evidence supports the hypotheses that the RARβ 2 gene is a tumor suppressor gene and the chemopreventive effects of retinoids are due to induction of RARβ 2. For example, a strategy commonly used to inactivate genes with tumor-suppressor properties, hypermethylation of the RARβ 2 gene is evident in colorectal cancer, small cell lung carcinoma and breast carcinoma. Moreover, higher methylation frequencies are also evident in the bone, brain, and lung metastasis stemming from breast carcinoma. RARβ 2 expression is reduced in many malignant tumors including breast carcinoma, head and neck, lung, esophagus, mammary gland, pancreas, and cervix. RARβ, and in particular RARβ 2, is thus currently used as a surrogate endpoint biomarker in different clinical prevention trials of various cancers. RARβ 2 ligands could be used alone or in combination with existing chemo- or radiation therapy. Synergistic cytotoxicity by combination treatment of selective retinoid RARα/β ligands with taxol (Paclitaxel) has already been demonstrated.

RARβ2 modulating compounds may be used to treat a variety of neurological disorders. For instance RARβ null mice exhibit locomotor defects related to dysfunction of the mesolimbic dopamine signaling pathway. Moreover these animals lack hippocampal long-term potentiation (LTP) and long-term depression (LTD), widely studied forms of synaptic plasticity. This results in substantial perfornance deficits in spatial learning and memory tasks. Interestingly, the expression of RARβ 2 in the brain strikingly overlaps that of the dopamine D1 and D2 receptors. Other animal studies reveal that deficiency in vitamin A, a precursor of retinoids, results in spatial learning and memory impairment as well as a loss in hippocampal long-term synaptic plasticity. Moreover, age-related relational memory deficit in mouse is associated with decreased expression of RARβ. Administration of retinoic acid, a pan RAR agonist, is accompanied by a complete restoration of the behavioral impairment and associated increase in RARβ expression. These effects can be antagonized by the use of a RAR antagonist. Finally, a growing body of evidence indicates RARβ 2 is involved in neurite outgrowth from peripheral and central nervous systems. Thus, RARβ 2 modulating compounds would be therapeutically relevant to the treatment of neurodegenerative disorders including Parkinson's and Alzheimer's diseases. Because of its involvement in cognitive function, neurological disorders where cognition is altered are also relevant, in particular schizophrenia. Finally, clinical data from the use of Isotretinoin has suggested an association with depression and suicide.

RARβ 2 modulating compounds may be used to treat a variety of hyper-proliferative and inflammatory disorders. Even though RARβ expression is below detection limits in the skin, RARβ 2 modulating compounds could act indirectly through transrepression of the activating protein 1 (AP1) complex, a heterodimeric transcription factor composed of Fos- and Jun-related proteins. AP1 is involved in the expression of metalloproteases, cytokines and other factors which play critical roles in the turnover of extracellular matrix, inflammation and hyperproliferation in diseases such as psoriasis, rheumatoid arthritis and in tumor metastases. The transrepressive effects of retinoids are mediated through a mechanism unrelated to transcriptional activation, involving the RAR-dependent control of transcription factors and cofactor assembly on AP1-regulated promoters. Relevant therapeutic indications include acne, psoriasis, photoaging and other dermatological disorders. RARβ2 modulating compounds may also be used to chronic inflammatory disorders and especially rheumatoid arthritis. For instance, retinoids through interaction with the AP-1 complex suppress collagenase gene expression. The fibroblast interstitial collagenase MMP-1, which degrades collagen, is thought to play a critical role in the degradation of the cartilage matric in arthritis. In animal models of arthritis, a RAR antagonist improves clinical and histological scores of arthritis.

RARβ 2 modulating compounds may be used to treat eye disorders/conditions. Vitamin A, the precursor of natural retinoids, is essential for the normal development and maintenance of the ocular surface. In the eye, RARβ mRNA transcripts are detectable in corneal stroma cells, conjunctival fibroblasts and corneal epithelial cells. RARβ expression is predominantly confined to the periocular mesenchyme and ciliary body. Moreover, retinoic acid further induces the expression of RARβ 3 in corneal and conjunctival fibroblasts. Knockout of RARβ 3 indicates that RARβ is the main RAR subtype involved in modulation of retinal cell populations. In chicken, retinoic acid through its actions on RARβ is associated with form-deprivation myopia.

### SUMMARY OF THE INVENTION

One embodiment disclosed herein includes a compound of Formula I

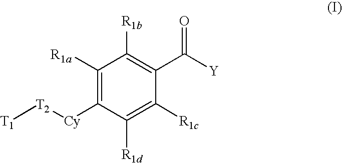

or a single isomer, mixture of isomers, racemic mixture of isomers, solvate, polymorph, metabolite, or pharmaceutically acceptable salt or prodrug thereof,
wherein:

R₁ₐ R_{1b}, R_{1c}, R_{1d} are independently selected from the group consisting of hydrogen, cyano, halogen, C₁₋₅ substituted or unsubstituted straight chained or branched alkyl, and substituted or unsubstituted cycloalkyl;

Cy is selected from the group consisting of substituted or unsubstituted aryl, substituted or unsubstituted heteroaryl, substituted or unsubstituted cycloalkyl, and substituted or unsubstituted heterocycle;

T₁ is selected from the group consisting of hydrogen, substituted or unsubstituted C₁-C₁₀ straight chained or branched alkyl, substituted or unsubstituted C₁-C₁₀ straight chained or branched alkenyl, substituted or unsubstituted C₁-C₁₀ straight chained or branched alkynyl, C₁-C₁₀ substituted or unsubstituted cycloalkyl, haloalkyl, —OR₂, —R₃OR₂, —OR₃OR₂, —N(R₂)(R₂ₐ), —C(═O)R₂, —C(═O)OR₂, —OC(═O)R₂, —C(═O)N(R₂)(R₂ₐ), —N(R₂)C(═O)(R₂ₐ), —N(R₂)C(═O)N(R₂ₐ) (R_{2b}), and —C═NN(R₂)(R₂ₐ);

T₂ is selected from the group consisting of C₁-C₁₀ substituted or unsubstituted straight chained or branched alkylene, C₁-C₁₀ substituted or unsubstituted straight chained or branched alkenylene, C₁-C₁₀ substituted or unsubstituted straight chained or branched acetylene, C₁-C₁₀ substituted or unsubstituted cycloalkylene, C₁-C₁₀ substituted or unsubstituted heterocycloalkylene, —OR₃—, —O—, —N(R₂)—, —C(O)—, —C(═O)O—, —OC(═O)—, —C(═O)N(R₂)—, —N(R₂)C(═O)—, —N(R₂)C(═O)N(R₂)—, and —C═NN(R₂)—;

Y is selected from the group consisting of —OH, —N(R₄ₐ, —C(═O)OH, OR₉, and —C(═O)OR₉;

R₄ and R₄ₐ are independently selected from the group consisting of hydrogen, —NH₂, —OH, —SO₂CH₃, C₁-C₁₀ substituted or unsubstituted alkyl, substituted or unsubstituted heteroaryl, substituted or unsubstituted aryl, and substituted or unsubstituted heterocycle, or R₄ and R₄ₐ together form a C₃-C₈ heteroaryl optionally substituted with —NR₄C(═O)R₂;

R₂, R₂ₐ, and R_{2b} are independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, substituted or unsubstituted C₃-C₉ cycloalkyl, substituted or unsubstituted C₅-C₇ cycloalkenyl, substituted or unsubstituted aryl, and substituted or unsubstituted heteroaryl;

R₃ is selected from the group consisting of substituted or unsubstituted C₁-C₁₀ straight chained or branched alkylene, substituted or unsubstituted C₂-C₆ straight chained or branched alkenylene, C₂-C₆ substituted or unsubstituted straight chained or branched alkynylene, substituted or unsubstituted C₃-C₇ cycloalkylene, CH₂CH₂CH═C(CHCH₂CH₂)₂, and substituted or unsubstituted C₅-C₇ cycloalkenylene; and

R₉ is selected from C₁-C₂₀ substituted or unsubstituted, straight chained or branched alkyl, substituted or unsubstituted heteroaryl, substituted or unsubstituted heterocycle, substituted or unsubstituted cycloalkyl, and substituted or unsubstituted aryl.

In an embodiment of the present invention Cy is selected from the group consisting of substituted or unsubstituted aryl, substituted or unsubstituted heteroaryl, substituted or unsubstituted cycloalkyl, and substituted or unsubstituted heterocycle.

In one embodiment, the prodrug of the compound of formula I is selected from an ester derivative, amide derivative, carbohydroxamic acid derivative, imidazole derivative, carbohydrazide derivative, or peptide derivative of the compound.

In one embodiment, Y is —OR₉ or —C(═O)OR₉.

In an embodiment, Cy is selected from the group consisting of:

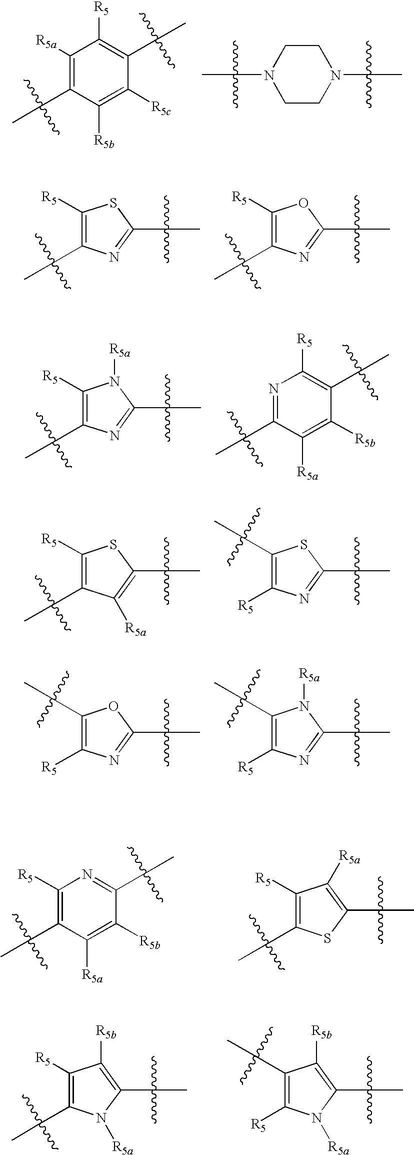

wherein:

R₅, R₅ₐ, R_{5b} and R_{5c} are independently selected from the group consisting of hydrogen, optionally substituted C₁-C₅ straight chained or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, —CN, —C(=Z)R₆, —C(=Z)OR₆, —C(=Z)N(R₆)R₆ₐ, —N(R₆)—C(=Z)R₆, —N(R₆)—C(=Z)N(R₆ₐ)R_{6b}, —NR₆)—S(═O)₂R₆ₐ, —OC(=Z)R₆, —S(═O)₂N(R₆)R₆ₐ, —S(O)N(R₆)R₆ₐ, —SO₂R₆, and —SR₆; and

R₆, R₆ₐ, and R_{6b} are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle.

In one embodiment, Cy is selected from the group consisting of:

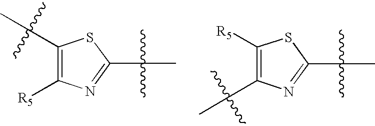

wherein:

R₅ is independently selected from the group consisting of hydrogen, C₁-C₅ straight chained or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, and —CN;

R₆ and R₆ₐ are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle.

In one embodiment, the compound according to Formula I is selected from the group comprising

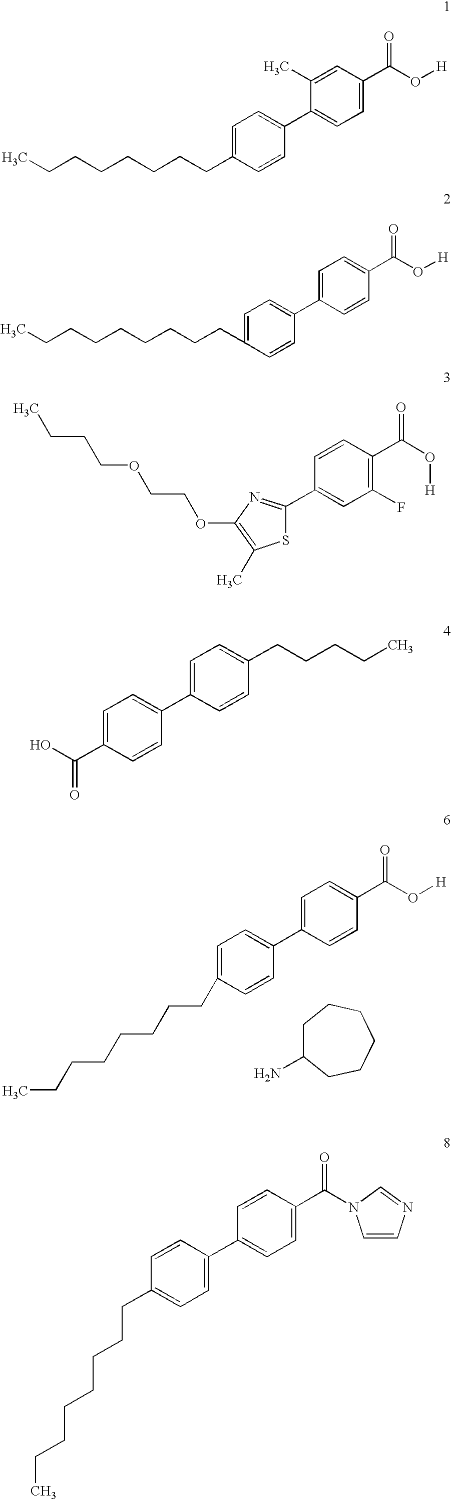

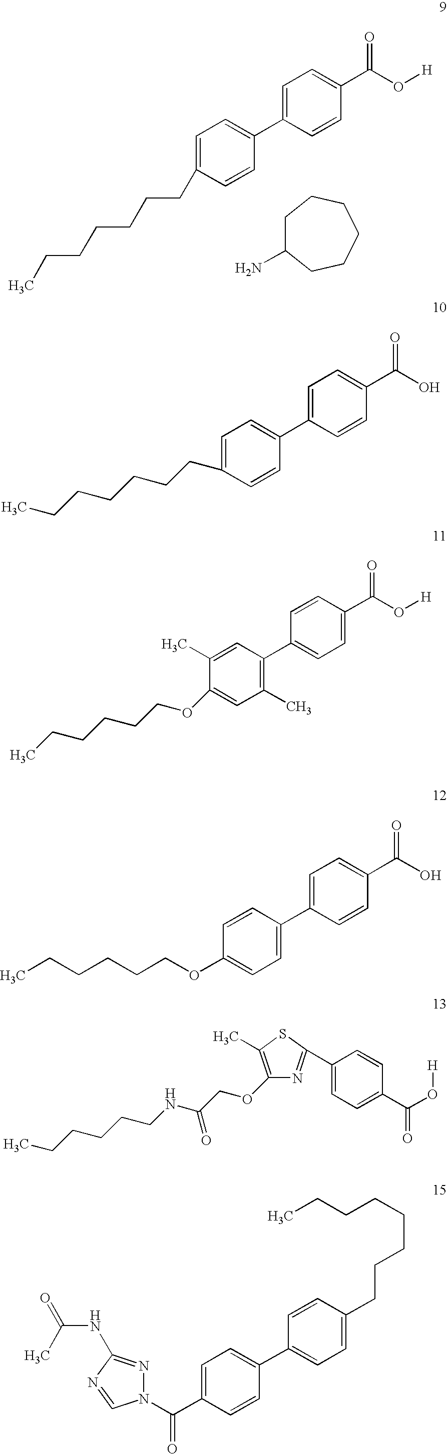

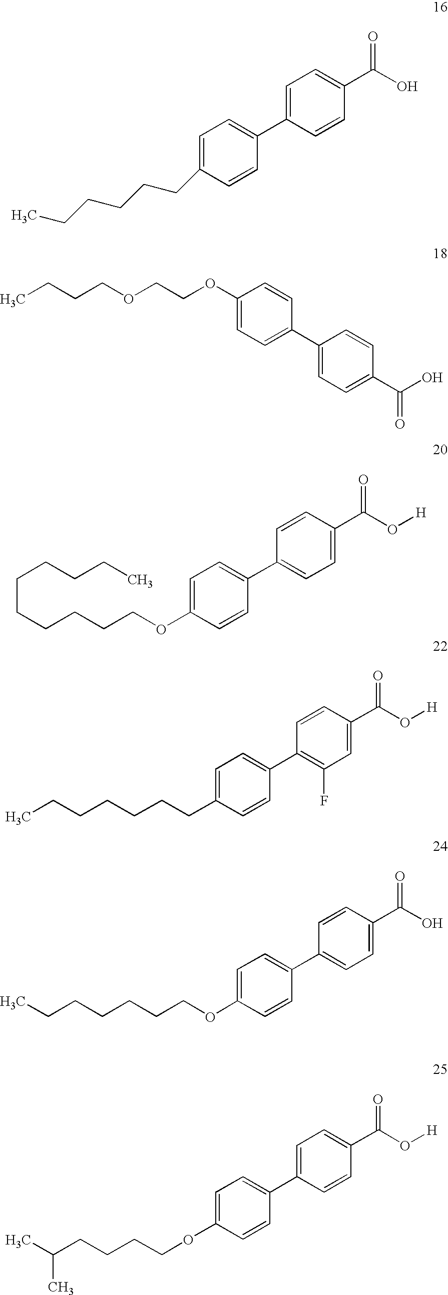

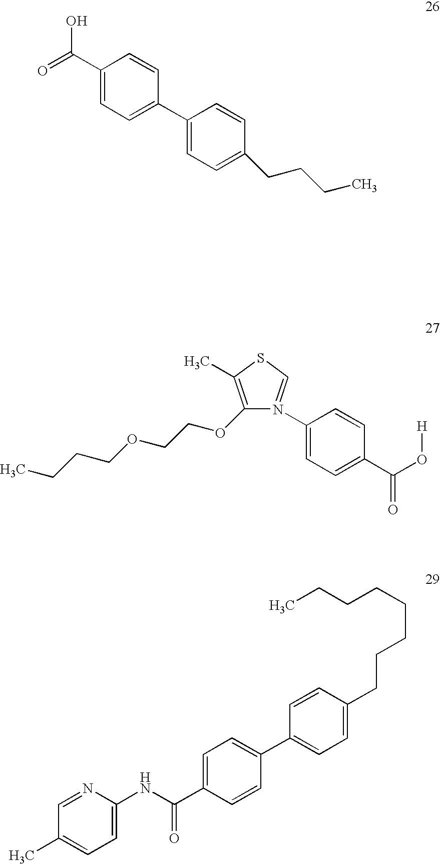

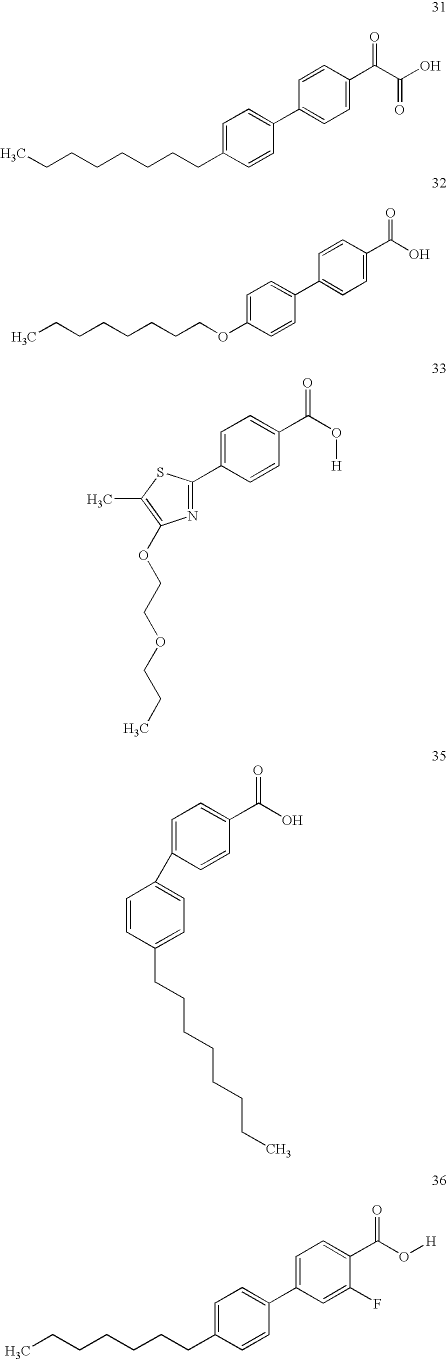

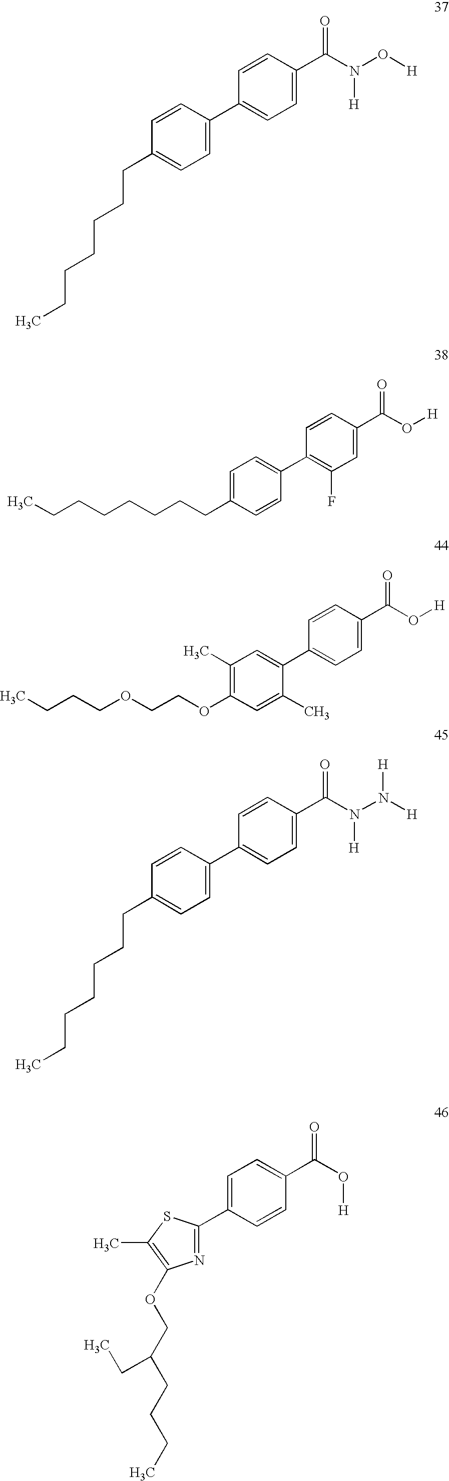

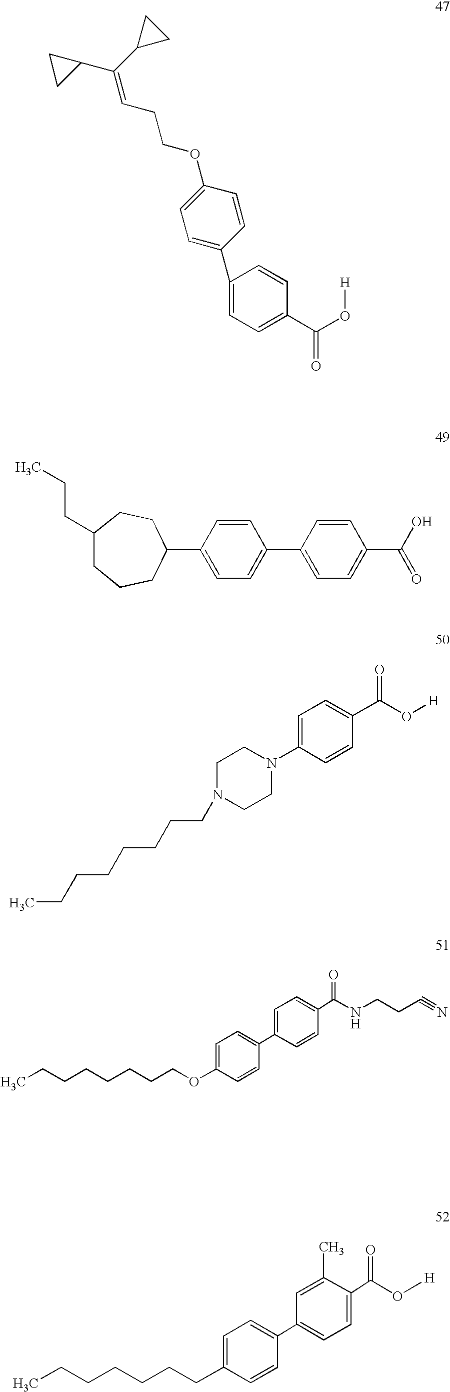

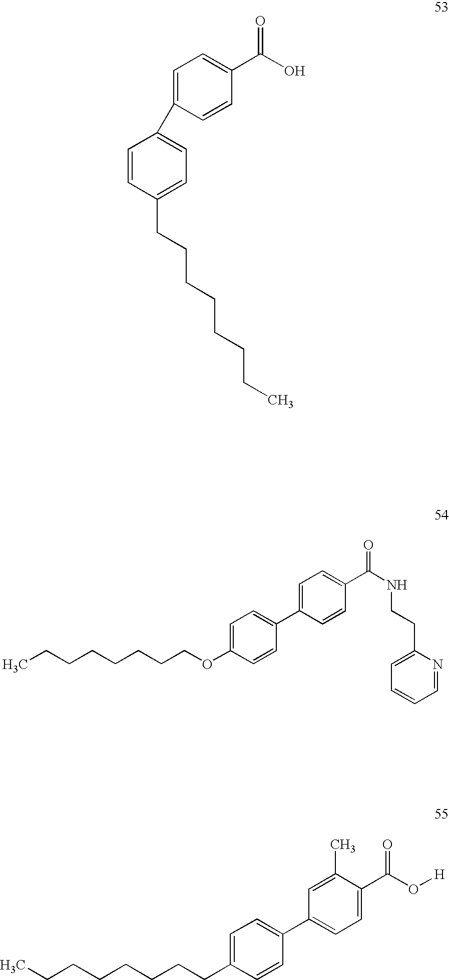

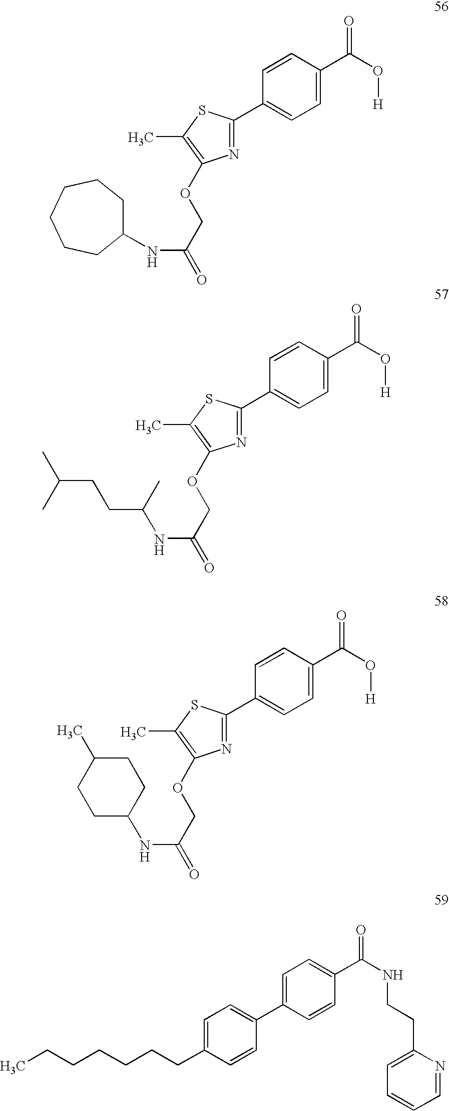

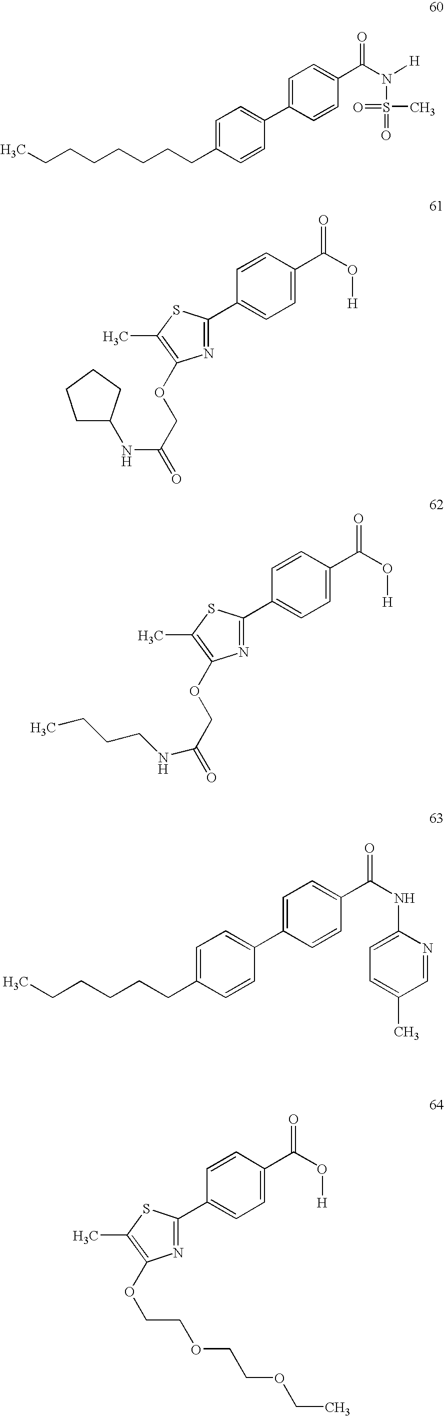

In an embodiment of the present invention preferred prodrugs are esters of Formula I, selected from the group consisting of:

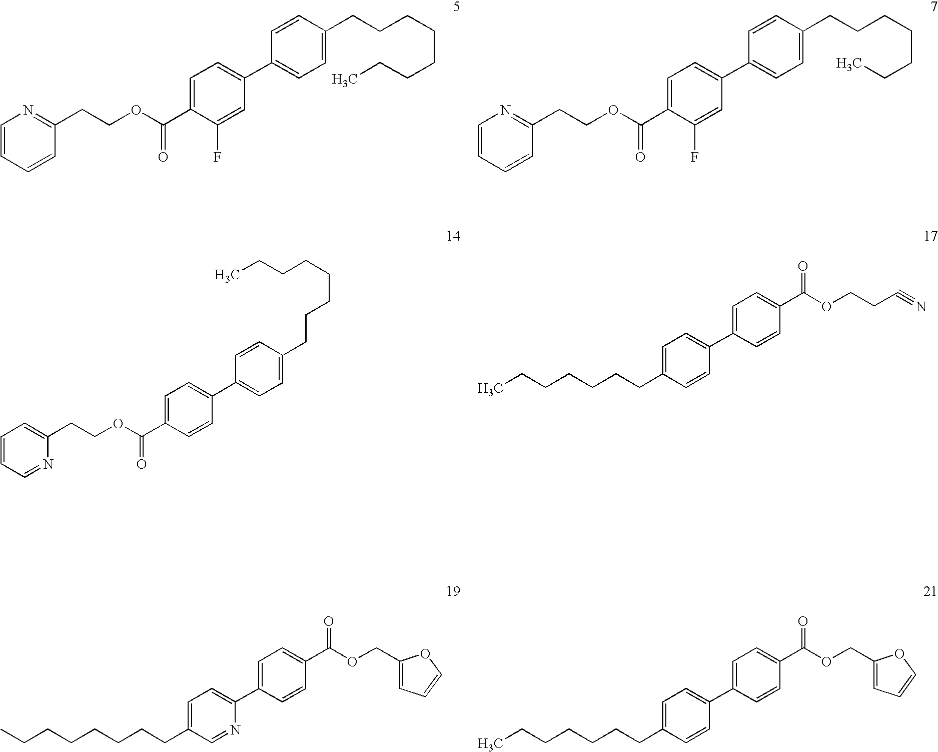

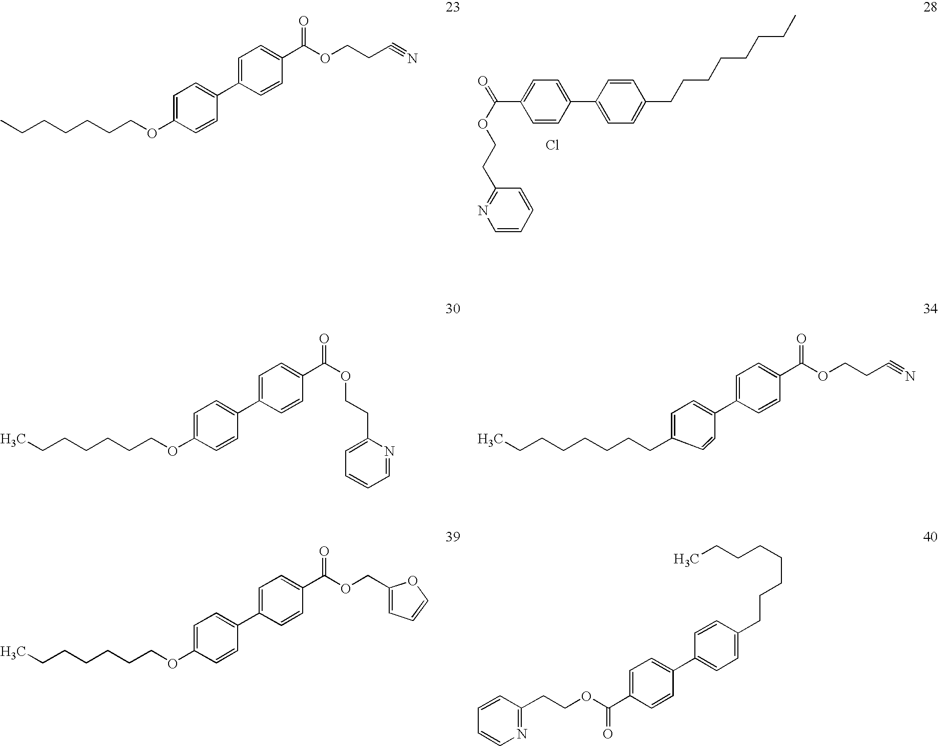

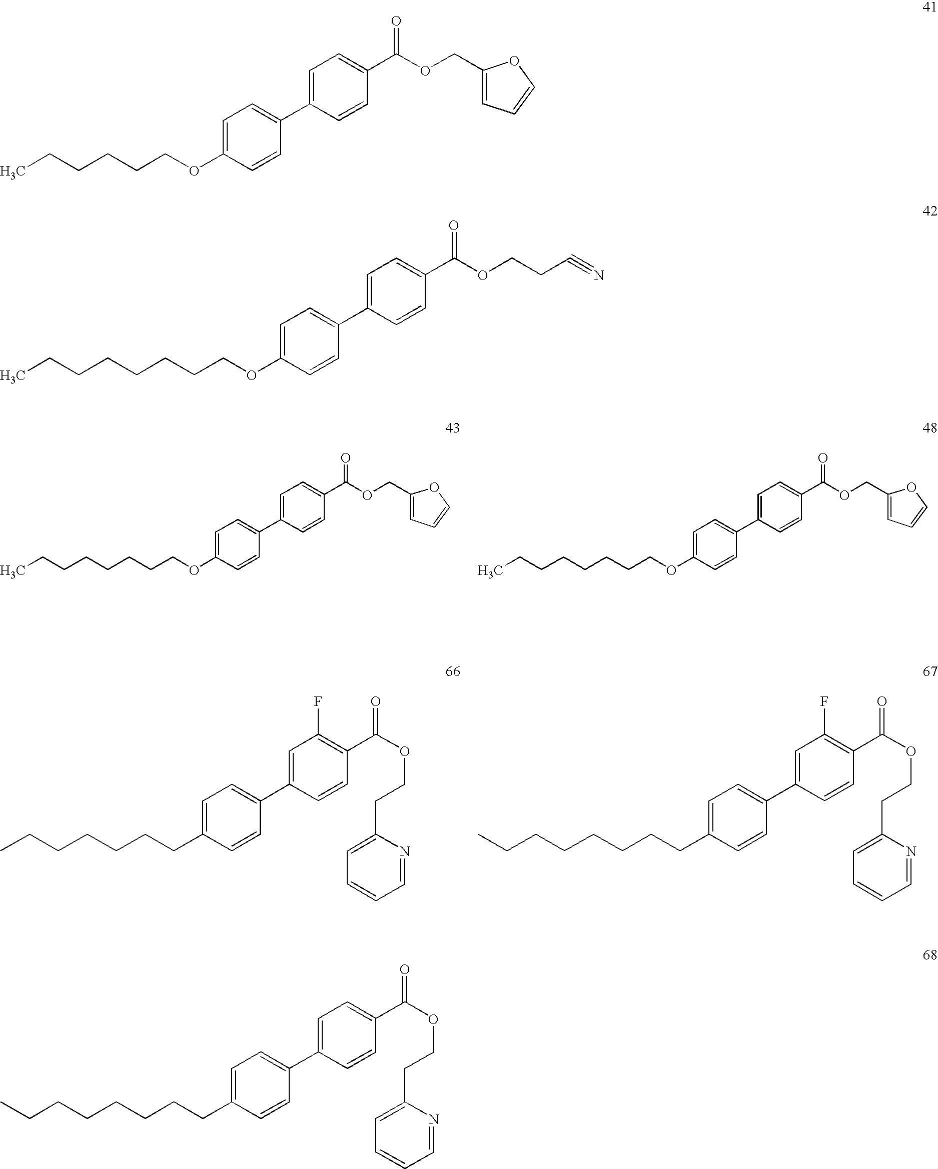

In one embodiment, the compound of Formula I has activity at RARβ receptor subtypes. In one embodiment, the compound has activity at the retinoic acid receptor subtype β isoform 2 (RARβ 2).

Another embodiment disclosed herein includes a method for the treatment of cancer or for alleviating cancer symptoms, comprising administering to a subject a therapeutically effective amount of at least one compound described above. An aspect of this embodiment includes administering at least one of the compounds described above in conjunction with at least one chemotherapeutic agent and/or radiation therapy. The term “in conjunction with” means given prior, concurrently, or subsequently to the other treatment. In one embodiment, the cancer is associated with malignant tumors. In one embodiment, the cancer is selected from the group consisting of breast carcinoma and tumors in head, neck, lung, esophagus, mammary gland, pancreas, or cervix.

An embodiment disclosed herein includes a method for the treatment of or for alleviating symptoms of a neurological disorder, comprising administering to a subject a therapeutically effective amount of at least one compound described above. In one embodiment, the neurological disorder is selected from the group consisting of performance deficits in spatial learning and memory tasks and age-related memory deficit. In one embodiment, the neurological disorder is a disorder wherein cognition is altered. In one embodiment, the neurological disorder is schizophrenia.

An embodiment disclosed herein includes a method for the treatment of or for alleviating symptoms of a neurodegenerative disorder, comprising administering to a subject a therapeutically effective amount of at least one compound described above. In one embodiment, the neurodegenerative disorder is Parkinson's disease or Alzheimer's disease.

Another embodiment disclosed herein includes a method for the treatment of or for alleviating symptoms of a neurodegenerative disorder, comprising administering to a subject a therapeutically effective amount of at least one compound described above. In one embodiment, the neurodegenerative disorder relates to a method for the treatment of neurodegenerative disorders where nerve regeneration is necessary after, e.g. a spinal cord injury, a stroke, damage to the cardiac musles, damage caused to myelin due to multiple sclerosis and damage to islet cells in diabetes.

Another embodiment disclosed herein includes a method for the treatment of or for alleviating symptoms of a hyperproliferative or inflammatory disorder, comprising administering to a subject a therapeutically effective amount of at least one compound described above. In one embodiment the inflammatory disorder is a chronic inflammatory disorder. In one embodiment, the inflammatory disorder is psoriasis or rheumatoid arthritis. One embodiment includes administering at least one compound of Formula I in combination with other treatments for inflammatory disorders such as corticorticoids, TNF modulaters, e.g., adalimumab, infliximab, etanercept, and T-cell activation modulators, such as efalizumab.

Another embodiment disclosed herein includes a method for treatment of or for alleviating symptoms of an eye disorder or an eye condition, comprising administering to a subject a therapeutically effective amount of at least one compound described above.

Another embodiment disclosed herein includes a method for treatment of or for alleviating symptoms of depression, comprising administering to a subject a therapeutically effective amount of at least one compound described above.

Another embodiment disclosed herein includes a method of identifying a compound which is an agonist, inverse agonist, or antagonist of one or more RARβ receptors, comprising contacting an RARβ receptor with at least one test compound described above and determining any change in activity level of the one or more RARβ receptors so as to identify the test compound as an agonist, inverse agonist, or antagonist of one or more RARβ receptors.

Another embodiment disclosed herein includes a pharmaceutical composition comprising a compound described above and at least one pharmaceutically acceptable adjuvant, excipient or carrier.

Another embodiment disclosed herein includes a compound described above for use in the treatment of cancer or for alleviation of cancer symptoms. In one embodiment, the compound is for use in combination with chemotherapy or radiation therapy.

In one embodiment, the cancer is associated with malignant tumors. In one embodiment, the cancer is selected from the group consisting of breast carcinoma and tumors in head, neck, lung, esophagus, mammary gland, pancreas, or cervix.

Another embodiment disclosed herein includes a compound described above for use in the treatment of or for alleviating symptoms of a neurological disorder. In one embodiment, the neurological disorder is a performance deficit in spatial learning and memory tasks and/or an age-related memory deficit. In one embodiment, the neurological disorder is a disorder wherein cognition is altered. In one embodiment, the neurological disorder is schizophrenia.

Another embodiment disclosed herein includes a compound described above for use in the treatment of or for alleviating symptoms of a neurodegenerative disorder. In one embodiment, the neurodegenerative disorder is Parkinson's disease or Alzheimer's disease.

Another embodiment disclosed herein includes a compound described above for use in the treatment of or for alleviating symptoms of a hyperproliferative or inflammatory disorder. In one embodiment, the inflammatory disorder is a chronic inflammatory disorder. In one embodiment, the inflammatory disorder is psoriasis or rheumatoid arthritis.

Another embodiment disclosed herein includes a compound described above for use in the treatment of or for alleviating symptoms of an eye disorder or an eye condition.

Another embodiment disclosed herein includes a compound described above for use in the treatment of or for alleviating symptoms of depression.

### DETAILED DESCRIPTION OF CERTAIN EMBODIMENTS

The present invention relates to a compound of Formula I

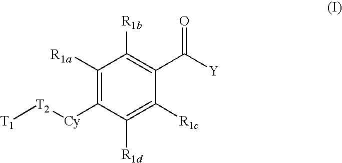

or a single isomer, mixture of isomers, racemic mixture of isomers, solvate, polymorph, metabolite, or pharmaceutically acceptable salt or prodrug thereof,
wherein:

R₁ₐ R_{1b}, R_{1c}, R_{1d} are independently selected from the group consisting of hydrogen, cyano, halogen, C₁₋₅ substituted or unsubstituted straight chained or branched alkyl, and substituted or unsubstituted cycloalkyl;

Cy is selected from the group consisting of substituted or unsubstituted aryl, substituted or unsubstituted heteroaryl, substituted or unsubstituted cycloalkyl, and substituted or unsubstituted heterocycle;

T₁ is selected from the group consisting of hydrogen, substituted or unsubstituted C₁-C₁₀ straight chained or branched alkyl, substituted or unsubstituted C₁-C₁₀ straight chained or branched alkenyl, substituted or unsubstituted C₁-C₁₀ straight chained or branched alkynyl, C₁-C₁₀ substituted or unsubstituted cycloalkyl, haloalkyl, —OR₂, —R₃OR₂, —OR₃OR₂, —N(R₂)(R₂ₐ), —C(═O)R₂, —C(═O)OR₂, —OC(═O)R₂, —C(═O)N(R₂)(R₂ₐ), —N(R₂)C(═O)(R₂ₐ), —N(R₂)C(═O)N(R₂ₐ)(R_{2b}), and —C═NN(R₂)(R₂ₐ);

T₂ is selected from the group consisting of C₁-C₁₀ substituted or unsubstituted straight chained or branched alkylene, C₁-C₁₀ substituted or unsubstituted straight chained or branched alkenylene, C₁-C₁₀ straight chained or branched acetylene, C₁-C₁₀ substituted or unsubstituted substituted or unsubstituted cycloalkylene, C₁-C₁₀ substituted or unsubstituted heterocycloalkylene, —OR₃—, —O—, —N(R₂)—, —C(═O)—, —C(═O)O—, —OC(═O)—, —C(═O)N(R₂)—, —N(R₂)C(═O)—, —N(R₂)C(═O)N(R₂)—, and —C═NN(R₂)—;

Y is selected from the group consisting of —OH, —NR₄R₄ₐ, —C(═O)OH, —OR₉, and —C(═O)OR₉;

R₄ and R₄ₐ are independently selected from the group consisting of hydrogen, —NH₂, —OH, —SO₂CH₃, C₁-C₁₀ substituted or unsubstituted alkyl, substituted or unsubstituted heteroaryl, substituted or unsubstituted aryl, and substituted or unsubstituted heterocycle, or R₄ and R₄ₐ together form a C₃-C₈ heteroaryl optionally substituted with —NR₄C(═O)R₂;

R₂, R₂ₐ, and R_{2b} are independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, substituted or unsubstituted C₃-C₉ cycloalkyl, substituted or unsubstituted C₅-C₇ cycloalkenyl, substituted or unsubstituted aryl, and substituted or unsubstituted heteroaryl;

R₃ is selected from the group consisting of substituted or unsubstituted C₁-C₁₀ straight chained or branched alkylene, substituted or unsubstituted C₂-C₆ straight chained or branched alkenylene, C₂-C₆ substituted or unsubstituted straight chained or branched alkynylene, C₃-C₇ substituted or unsubstituted cycloalkylene, CH₂CH₂CH═C(CHCH₂CH₂)₂, and C₅-C₇ substituted or unsubstituted cycloalkenylene; and

R₉ is selected from C₁-C₂₀ substituted or unsubstituted, straight chained or branched alkyl, substituted or unsubstituted heteroaryl, substituted or unsubstituted heterocycle, substituted or unsubstituted cycloalkyl, and substituted or unsubstituted aryl.

In some embodiments, certain compounds of Formula I are prodrugs that readily metabolize to other compounds according to Formula I. Some embodiments include prodrugs that are derivatives of compounds of Formula I. In some embodiments of the present invention prodrugs are ester derivatives, amide derivatives, carbohydroxamic acid derivative, imidazole derivatives, carbohydrazide derivative, or peptide derivatives of the compound according to Formula I. Suitable prodrugs include compounds of Formula I and derivatives of compounds according to Formula I that are metabolically labile, e.g. hydrolysable, in vivo. In some embodiments, such prodrugs include compounds of Formula I where Y₁ is selected from —OR₉ and —C(—O)OR₉.

In a preferred embodiment of the compound according to the invention Cy is selected from the group consisting of substituted or unsubstituted aryl, substituted or unsubstituted heteroaryl, substituted or unsubstituted cycloalkyl, and substituted or unsubstituted heterocycle.

In the above-mentioned embodiments Cy is preferably selected from the group consisting of:

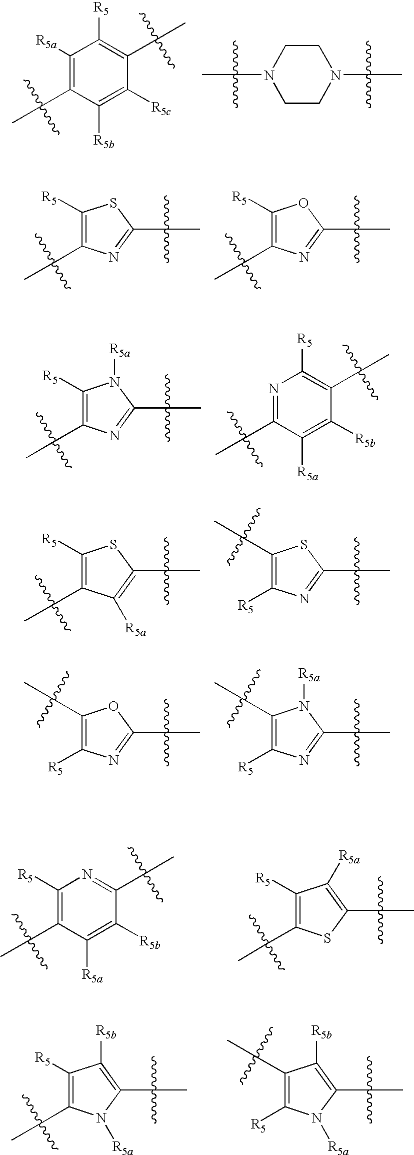

wherein:

R₅, R₅ₐ, R_{5b} and R_{5c} are independently selected from the group consisting of hydrogen, substituted or unsubstituted C₁-C₅ straight chained or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, —CN, —C(═O)R₆, —C(═O)OR₆, —C(═O)N(R₆)R₆ₐ, —N(R₆)—C(═O)R₆ₐ, —N(R₆)—C(═O)N(R₆ₐ)R_{6b}, —N(R₆)—S(═O)₂R₆ₐ, —OC(═O)R₆, —S(═O)₂N(R₆)R₆ₐ, —S(═O)N(R₆)R₆ₐ, and —SO₂R₆ and —SR₆; and

R₆, R₆ₐ and R_{6b} are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle

In a preferred embodiment of the compound of the invention Cy is selected from the group consisting of:

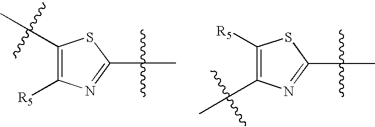

In certain embodiments, the compound of Formula I are selected from the group consisting of the following compounds:

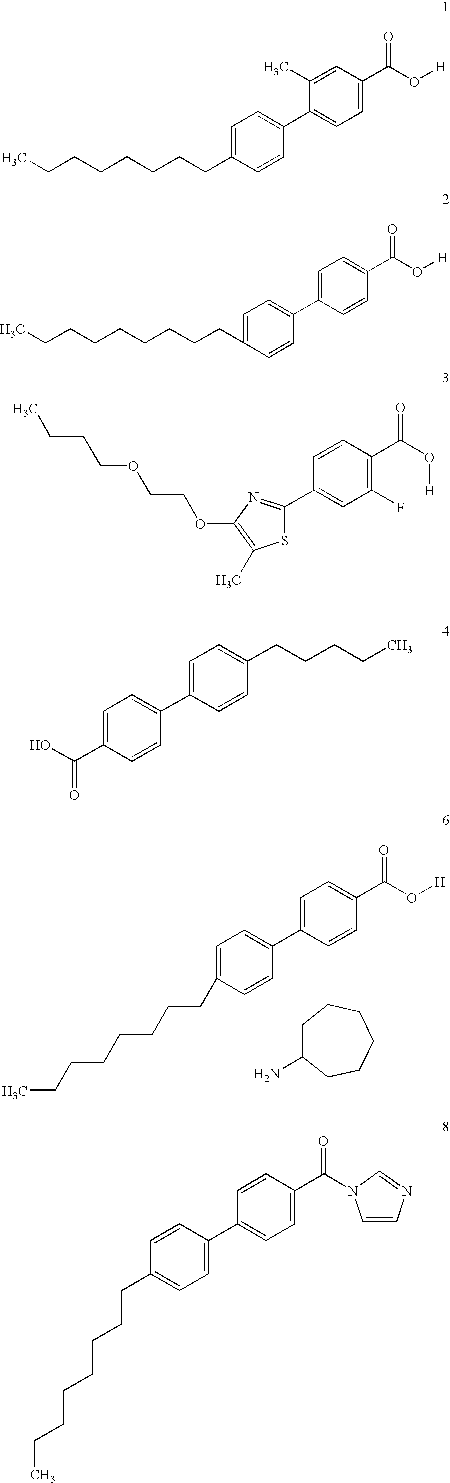

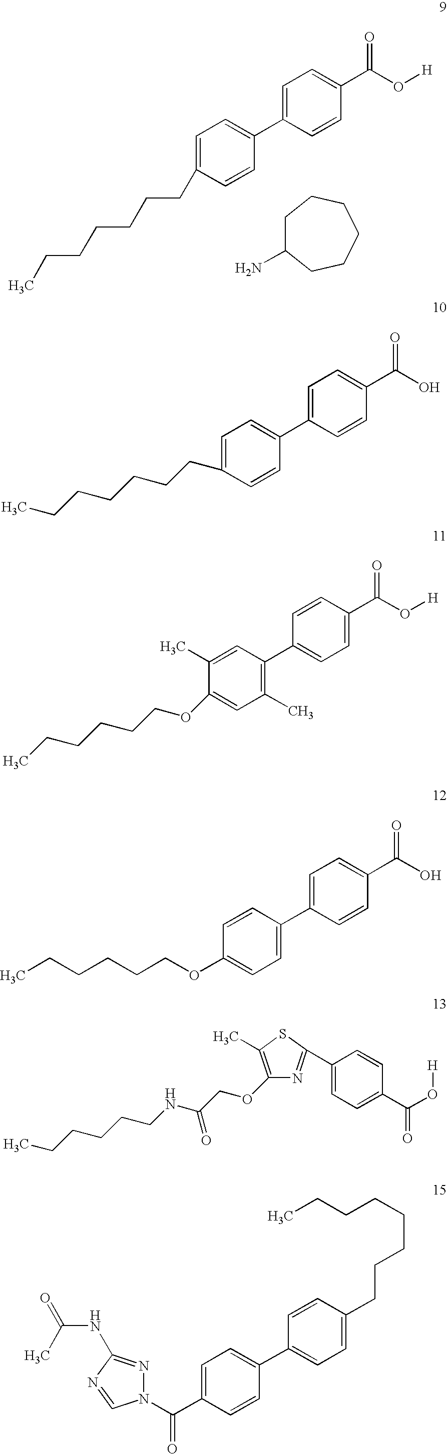

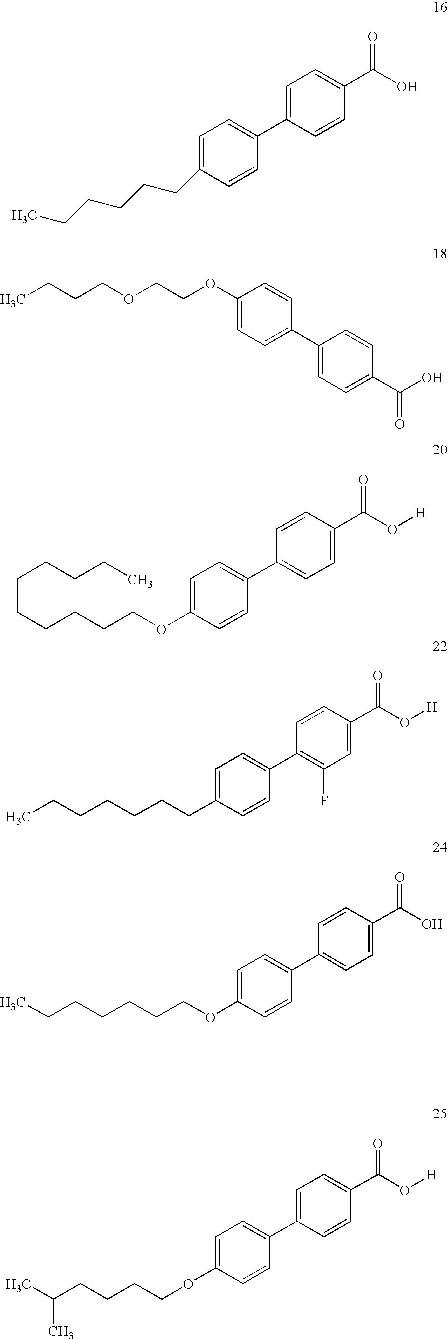

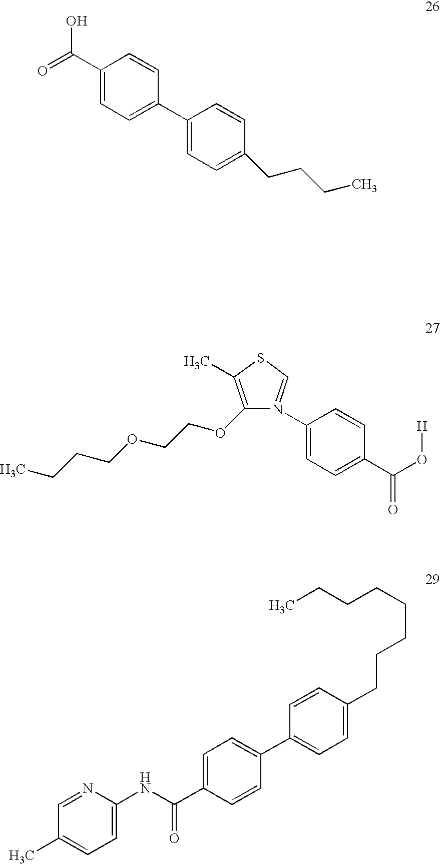

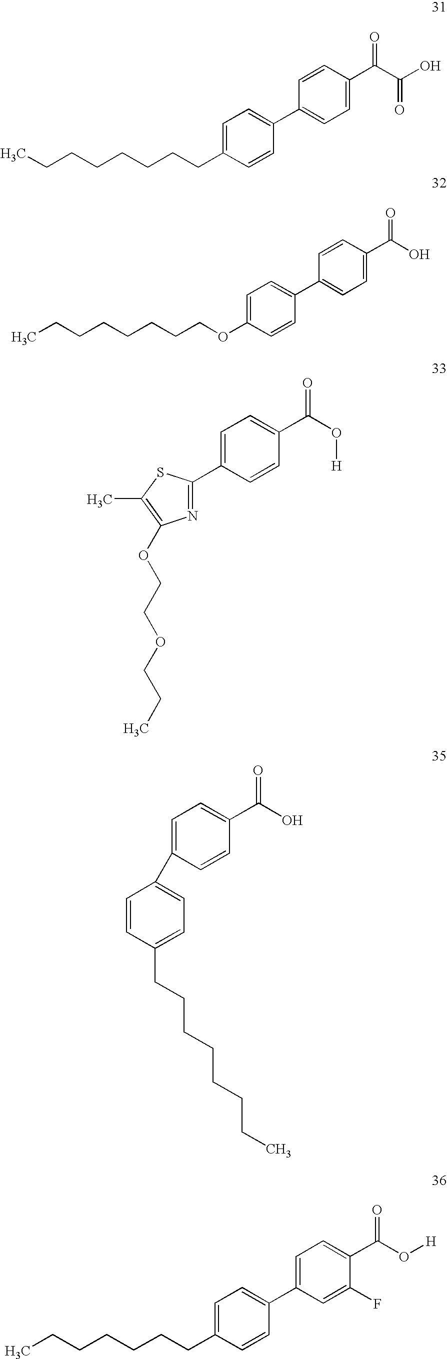

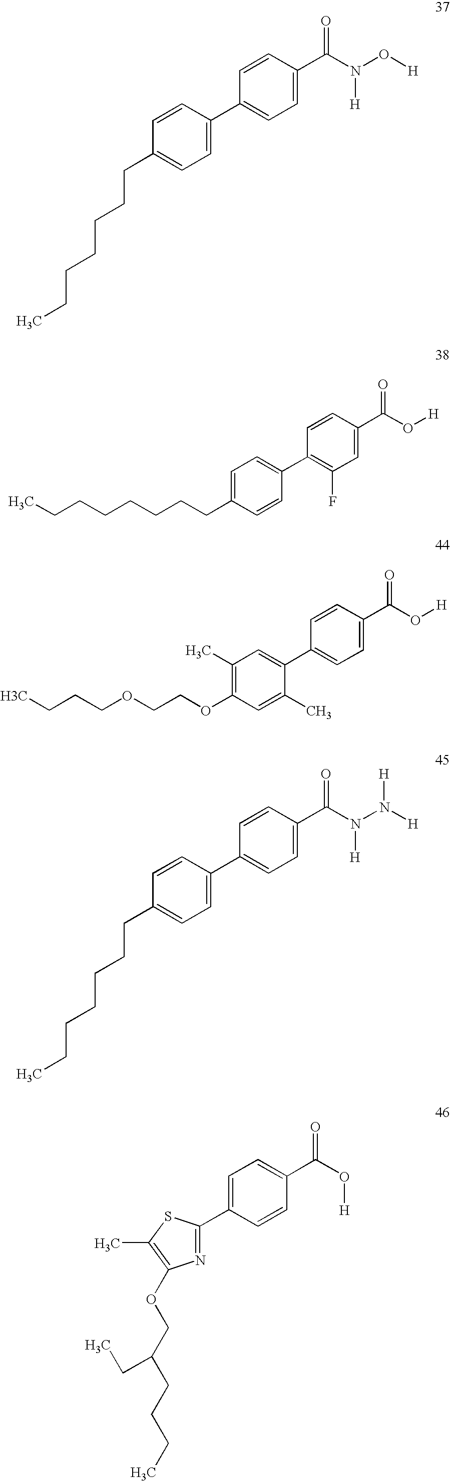

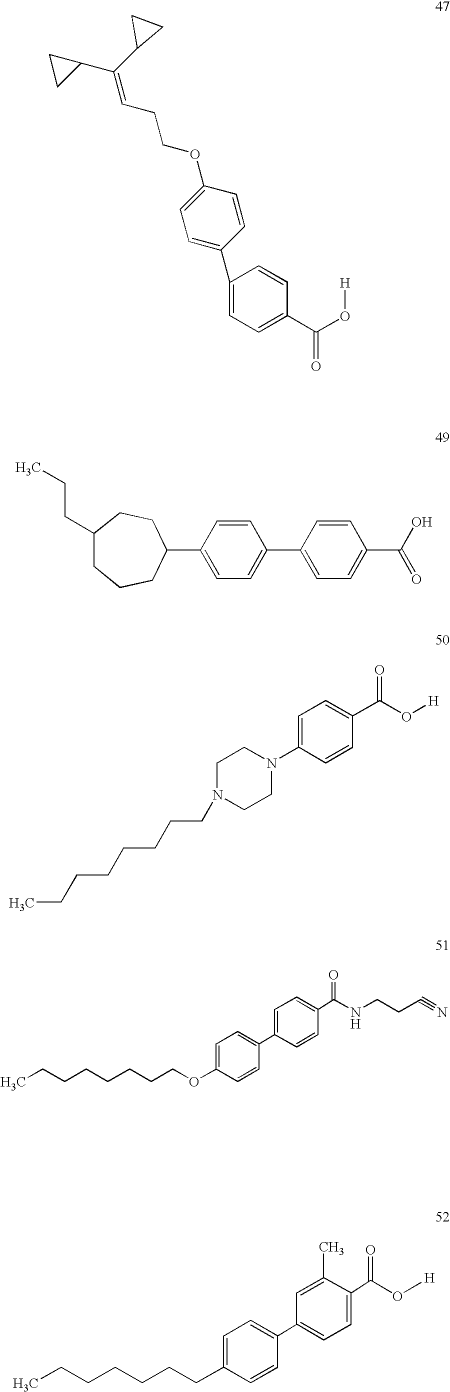

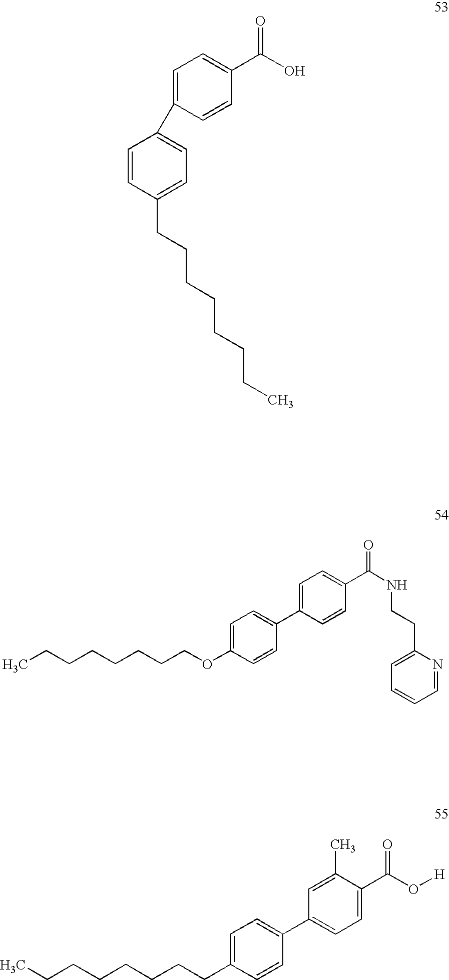

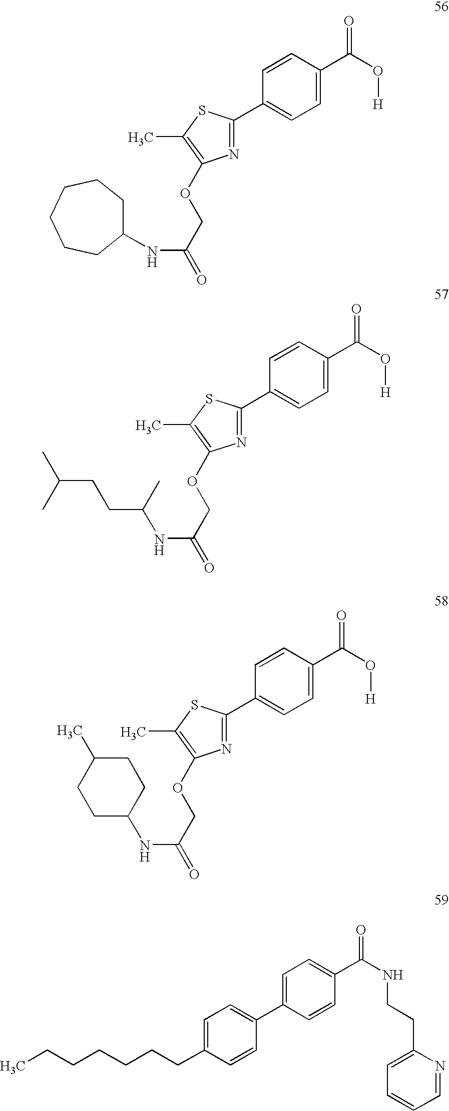

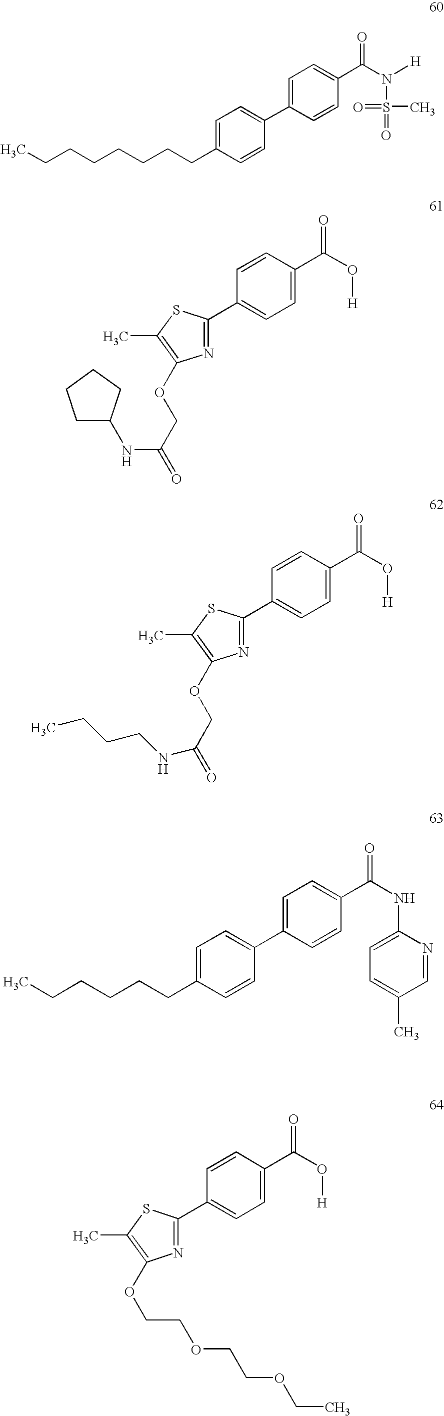

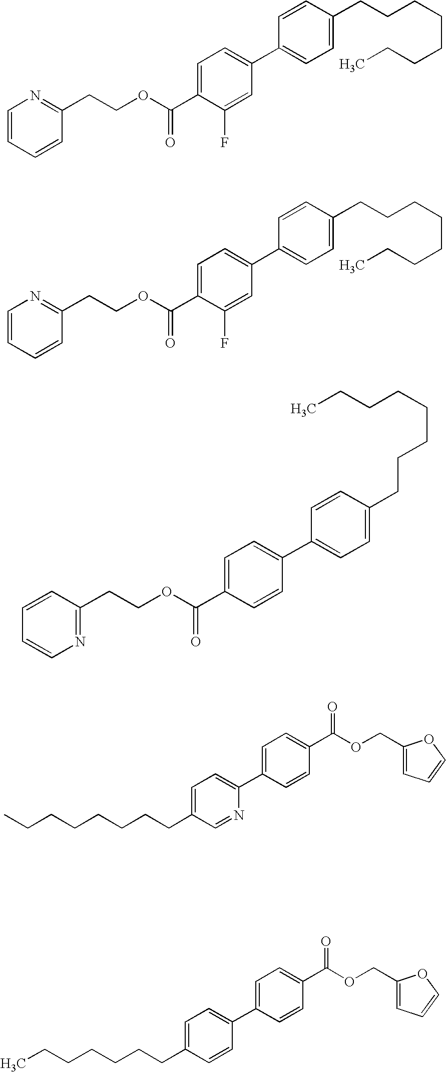

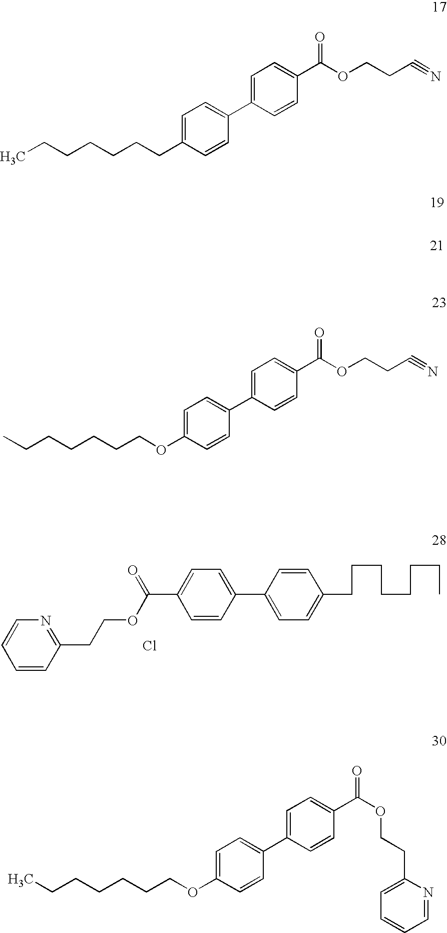

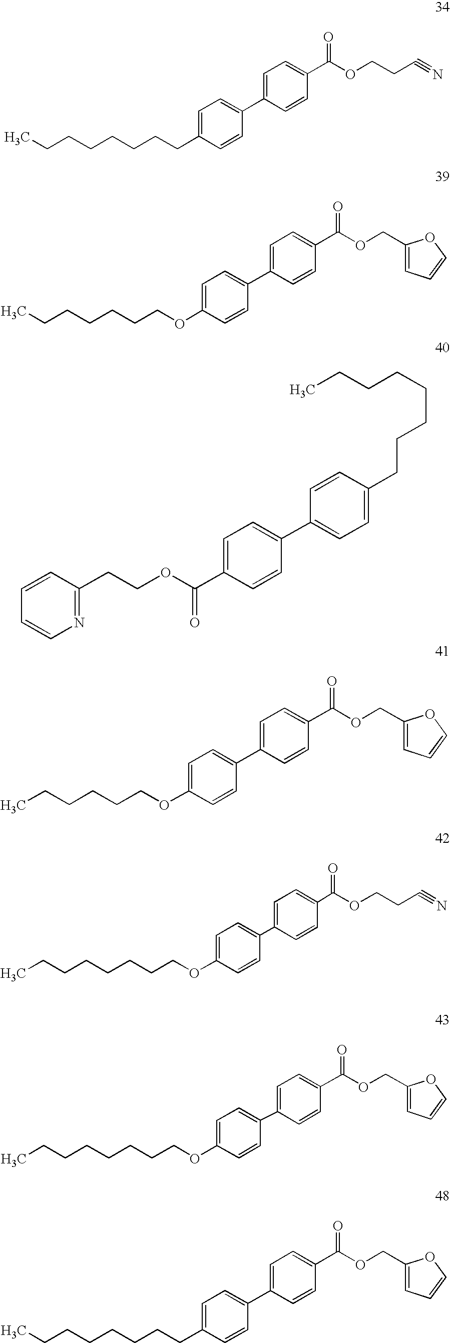

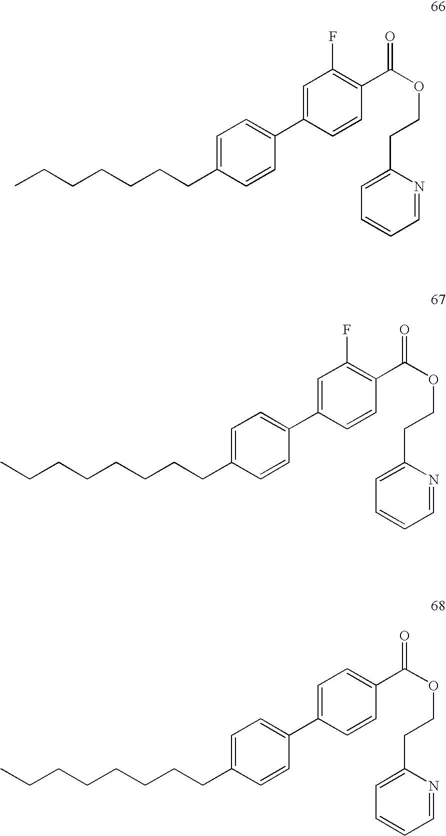

In one aspect, the present invention relates to a method for treatment of cancer or for alleviating cancer symptoms comprising administering to a subject an effective amount of at least one compound of Formula I. The method could be used alone or in combination with existing chemo- or radiation therapy.

In one aspect, the invention relates to a method for treatment of cancer wherein the cancer is associated with malignant tumors including breast carcinoma, head and neck, lung, esophagus, mammary gland, pancreas, and cervix.

In one aspect, the present invention relates to a method to treat or alleviate symptoms of a variety of neurological disorders which includes but is not limited to performance deficits in spatial learning and memory tasks and age-related memory deficit, comprising administering to a subject an effective amount of at least one compound of Formula I.

In one aspect, the present invention relates to a method for the treatment of neurodegenerative disorders including Parkinson's and Alzheimer's diseases comprising administering to a subject an effective amount of at least one compound of Formula I.

In one aspect, the present invention relates to a method for the treatment of neurodegenerative disorders where nerve regeneration is necessary after, e.g. a spinal cord injury, a stroke, damage to the cardiac musles, damage caused to myelin in multiple sclerosis and damage to the islet cells in diabetes comprising administering to a subject an effective amount of at least one compound of Formula I.

In one aspect, the present invention relates to a method for alleviating symptoms of neurological disorders such as where cognition is altered e.g., schizophrenia, wherein said method comprises administering to a subject an effective amount of at least one compound of Formula I.

In one aspect, the present invention relates to a method for treatment of a variety of hyperproliferative and inflammatory disorders comprising administering to a subject an effective amount of at least one compound of Formula I.

In one aspect, the present invention relates to a method for treatment of inflammation when associated with chronic inflammatory disorders, e.g. rheumatoid arthritis.

In one aspect, the present invention relates to a method to treat eye disorders/conditions comprising administering to a subject an effective amount of at least one compound of Formula I.

In one aspect, the present disclosure is related to a method to identify a compound which is an agonist, inverse agonist or antagonist of one or more RARβ receptors, the method comprising: contacting a RARβ receptor with at least one test compound of Formula I; and determining any change in activity level of the one or more RARβ receptors so as to identify a test compound, which is an agonist, inverse agonist or antagonist of one or more RARβ receptors.

In certain embodiments, the above methods for alleviating different diseases and conditions further comprise the step of identifying a subject in need of alleviating symptoms of cancer, neurological disorders, neurodegenerative disorders, hyperproliferative and inflammatory disorders, eye disorders/conditions prior to the contacting step.

In some embodiments, the compound of Formula I modulates activity at the RARβ receptor subtypes.

Compounds or prodrugs, 1-68, disclosed herein, represent preferred compounds or prodrugs to be used in the methods disclosed herein.

In one aspect, disclosed herein is a method of identifying a compound which is an agonist, inverse agonist or antagonist of a RARβ receptor, the method comprising culturing cells that express the RARβ receptor; incubating the cells with at least one compound of Formula I as defined herein; and determining any increase or decrease in activity of the RARβ receptor so as to identify a compound of Formula I which is an agonist, inverse agonist or antagonist of a RARβ receptor.

In certain embodiments, the cultured cells overexpress the RARβ receptor In other embodiments, the identified agonist, inverse agonist or antagonist is selective for the RARβ receptor. In order to decide if a compound has activity at RARβ receptor subtypes or has activity at the retinoic acid receptor subtype β isoform 2 (RARβ 2), the test method disclosed below in Example 40 may be used. When using this test, a compound is considered to have an activity at the receptor if the pEC50 is ≧5.0 and the % Eff is ≧25.

In one aspect, the present invention relates to a pharmaceutical composition comprising a compound of Formula I as described herein, and a physiologically acceptable component such as a carrier, a diluent, or an excipient, or a combination thereof.

In one embodiment, compounds disclosed herein induce neuronal differentiation. For example, compounds of Formulae 6 and 68 have been discovered to induce neuronal differentiation in NTERA-2 Cells.

The term “pharmaceutically acceptable salt” refers to a formulation of a compound that does not cause significant irritation to a subject to which it is administered and does not abrogate the biological activity and properties of the compound. Pharmaceutical salts can be obtained by reacting a compound disclosed herein with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound disclosed herein with a base to form a salt such as an ammonium salt, an alkali metal salt, such as a sodium or a potassium salt, an alkaline earth metal salt, such as a calcium or a magnesium salt, a salt of organic bases such as dicyclohexylatnine, N-methyl-D-glucamine, tris(hydroxymethyl)nethylamine, and salts with amino acids such as arginine, lysine, and the like.

According to the present invention the term “ester” refers to a chemical moiety with formula —(R)ₙ—COOR′, where R and R′ are independently selected from the group consisting of alkyl, cycloalkyl, aryl, heteroaryl (bonded through a ring carbon) and heterocyclic (bonded through a ring carbon), and where n is 0 or 1.

An “amide” is a chemical moiety with formula —(R)ₙ—C(O)NHR′ or —(R)ₙ—NHC(O)R′, where R and R′ are independently selected from the group consisting of alkyl, cycloalkyl, aryl, heteroaryl (bonded through a ring carbon) and heterocyclic (bonded through a ring carbon), and where n is 0 or 1. An amide may be an amino acid or a peptide molecule attached to a molecule of disclosed herein, thereby forming a prodrug.

In some embodiments, any amine, hydroxy, or carboxyl side chain on the compounds disclosed herein may be esterified or amidified. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999, which is incorporated by reference herein in its entirety.

In some embodiments, any ester, amide or any other carboxylic acid derivative on the compounds disclosed herein can be hydrolyzed. The procedures and specific groups to be used to achieve this end is known to those of skill in the art and can readily be found in reference sources such as Greene and Wuts, Protective Groups in Organic Synthesis, 3^{rd} Ed., John Wiley & Sons, New York, N.Y., 1999.

A “prodrug” refers to an agent that is converted into the parent drug in vivo. Prodrugs are often useful because, in some situations, they may be easier to administer than the parent drug, they may for instance be metabolically labile or hydrolysable. They may, for instance, be bioavailable by oral administration whereas the parent is not. The prodrug may also have improved solubility in pharmaceutical compositions over the parent drug. An example, without limitation, of a prodrug would be a compound disclosed herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

The term “aromatic” refers to an aromatic group which has at least one ring having a conjugated pi electron system and includes both carbocyclic aryl (e.g., phenyl) and heterocyclic aryl groups (e.g., pyridine). The term includes monocyclic or fused-ring polycyclic (i.e., rings which share adjacent pairs of carbon atoms) groups. The term “carbocyclic” refers to a compound which contains one or more covalently closed ring structures, and that the atoms forming the backbone of the ring are all carbon atoms. The term “heteroaromatic” or “heteroaryl” refers to an aromatic group, which contains at least one heterocyclic ring, which may be optionally substituted. In various embodiments, these groups may be substituted or unsubstituted.

Examples of aryl ring and fused aryl include, but are not limited to, benzene, and substituted benzene, such as toluene, aniline, xylene, and the like, naphthalene and substituted naphthalene, and azulene.

Examples of heteroaryl ring include, but are not limited to, furan, thiophene, pyrrole, oxazole, thiazole, imidazole, imidazoline, pyrazole, pyrazoline, quinoline, indole, isoxazole, isothiazole, triazole, thiadiazole, pyran, pyridine, pyridazine, pyrimidine, pyrazine, piperazine and triazine.

The terms “heterocyclic” or “heterocycle” refers to saturated or unsaturated rings with from one to twenty carbon atoms, which contains at least one heteroatom selected from nitrogen, oxygen, and sulfur, optionally condensed with another aromatic or non-aromatic ring. The ring may be optionally substituted by one or more groups, the same or different. Optionally the ring may be bicyclic. Examples of heterocyclic rings are: pyllodidine, pyrroline, piperidine, imidazolidine, pyrazolidine, dihydropiperidine, dihydropyridine, piperazine, morpholine, thiomorpholine, thiazine and indoline. In various embodiments, these groups may be substituted or unsubstituted.

The term “cycloalkyl” refers to the univalent group derived from monocyclic hydrocarbons (with or without side chains) (E.g. cyclobutane) by removal of a hydrogen atom from the ring. In various embodiments, these groups may be substituted or unsubstituted.

Examples of cycloalkyl groups, are cyclopropyl, cyclobutyl, cyclopentyl, cyclohexyl.

The term “cycloalkene” and “cycloalkynes” refers to unsaturated monocyclic hydrocarbons having one endocyclic double or one triple bond, respectively. Those having more than one such multiple bond are cycloalkadienes, cycloalkatrienes, etc. The inclusive terms for any cyclic hydrocarbons having any number of such multiple bonds are cyclic olefins or cyclic acetylenes. In various embodiments, these groups may be substituted or unsubstituted.

As used herein, the term “alkyl” refers to an aliphatic hydrocarbon group. The alkyl moiety may be a “saturated alkyl” group, which means that it does not contain any alkene or alkyne moieties. The alkyl moiety may also be an “unsaturated alkyl” moiety, which means that it contains at least one alkene or alkyne moiety. The alkyl moiety, whether saturated or unsaturated, may be branched, straight chain, or cyclic. An “alkene” moiety refers to a group consisting of at least two carbon atoms and at least one carbon-carbon double bond, and an “alkyne” moiety refers to a group consisting of at least two carbon atoms and at least one carbon-carbon triple bond. An “alkenyl” moiety refers to a linear or branched alkenyl group, e.g. ethenyl, propenyl, butenyl. An “alkynyl” moiety refers to a branched or unbranched alkynyl group, e.g. ethynyl, propargyl. An “acetylene” moiety refers to acyclic (branched or unbranched) and cyclic (with or without side chain) hydrocarbons having one or more carbon-carbon triple bonds. An “alkylidene” moiety refers to the divalent groups formed from alkanes by removal of two hydrogen atoms from the same carbon atom the free valencies of which are part of a double bond, such as ═CR′R″. Alkylidene groups include, but are not limited to methylidene (═CH₂) and ethylidene (═CHCH₃).

The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds disclosed herein may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl.

The alkyl group may be substituted or unsubstituted. When substituted, the substituent group(s) is(are) one or more group(s) individually and independently selected from cycloalkyl, aryl, heteroaryl, heterocycle, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, tribalomethanesulfonyl, and amino, including mono- and di-substituted amino groups, and the protected derivatives thereof. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like. Wherever a substituent is described as being “optionally substituted” that substituent may be substituted with one of the above substituents.

The term “alkylene” refers to an alkyl group, as defined here, which is a biradical and is connected to two other moieties. Thus, methylene (—CH₂—), ethylene (—CH₂CH₂—), propylene (—CH₂CH₂CH₂—), isopropylene (—CH₂—CH(CH₃)—), and isobutylene (—CH₂—CH(CH₃)—CH₂—) are examples, without limitation, of an alkylene group. In various embodiments, these groups may be substituted or unsubstituted.

The term “alkenylene” refers to an alkylene group, as defined here, that contains in the straight or branched hydrocarbon chain and one or more double bonds. The group is a bivalent radical derived by removing a hydrogen atom from each of the terminal carbon atoms. If only one double bond is present in the hydrocarbon chain is it represented by the formula —(CₙH₂ₙ₋₂)—. An alkenylene group of this invention may be unsubstituted or substituted. When substituted, the substituent(s) may be selected from the same groups disclosed above with regard to alkyl group substitution. Alkenylene groups include, but are not limited to, propenylene —IC═C═CH— and vinylene (ethenylene) —HC═CH—. In various embodiments, these groups may be substituted or unsubstituted.

The term “alkoxy” and “alkylthio” refers to RO— and RS—, in which R is an alkyl. In various embodiments, these groups may be substituted or unsubstituted.

The substituent “R” appearing by itself and without a number designation refers to a substituent selected from the group consisting of alkyl, cycloalkyl, aryl, heteroaryl (bonded through a ring carbon) and heteroalicyclic (bonded through a ring carbon).

An “O-carboxy” group refers to a RC(═O)O— group, where R is as defined herein. In various embodiments, these groups may be substituted or unsubstituted.

A “C-carboxy” group refers to a —C(═O)OR groups where R is as defined herein. In various embodiments, these groups may be substituted or unsubstituted.

An “acetyl” group refers to a —C(═O)CH₃ group.

A “trihalomethanesulfonyl” group refers to a X₃CS(═O)₂— group where X is a halogen.

A “cyano” group refers to a —CN group.

An “isocyanato” group refers to a —NCO group. In various embodiments, these groups may be substituted or unsubstituted.

A “thiocyanato” group refers to a —CNS group.

An “isothiocyanato” group refers to a —NCS group.

A “sulfinyl” group refers to a —S(═O)—R group, with R as defined herein. In various embodiments, these groups may be substituted or unsubstituted.

A “S-sulfonamido” group refers to a —S(—O)₂NR group, with R as defined herein. In various embodiments, these groups may be substituted or unsubstituted.

A “N-sulfonamido” group refers to a RS(═O)₂NH— group with R as defined herein. In various embodiments, these groups may be substituted or unsubstituted.

A “trihalomethanesulfonamido” group refers to a X₃CS(═O)₂NR— group with X and R as defined herein. In various embodiments, these groups may be substituted or unsubstituted.

An “O-carbamyl” group refers to a —OC(═O)—NR group-with R as defined herein. In various embodiments, these groups may be substituted or unsubstituted.

An “N-carbamyl” group refers to a ROC(═O)NH— group, with R as defined herein. In various embodiments, these groups may be substituted or unsubstituted.

An “O-thiocarbamyl” group refers to a —OC(═S)—NR group with R as defined herein. In various embodiments, these groups may be substituted or unsubstituted.

An “N-thiocarbamyl” group refers to an ROC(═S)NH— group, with R as defined herein. In various embodiments, these groups may be substituted or unsubstituted.

A “C-amido” group refers to a —C(═O)—NR₂ group with R as defined herein. In various embodiments, these groups may be substituted or unsubstituted.

An “N-amido” group refers to a RC(—O)NH— group, with R as defined herein. In various embodiments, these groups may be substituted or unsubstituted.

The term “haloalkyl” refers to an alkyl group where one or more of the hydrogen atoms are replaced by halogen. Such groups include but are not limited to, chloromethyl, fluoromethyl, difluoromethyl, trifluoromethyl and 1-chloro-2-fluoromethyl, 2-fluoroisobutyl. In various embodiments, these groups may be substituted or unsubstituted.

Where the numbers of substituents not are specified (e.g. haloalkyl) there may be one or more substituents present. For example “haloalkyl” may include one or more of the same or difference halogens. As another example “C₁-C₃ alkoxy phenyl” may include one or more of the same of different alkoxy groups containing one, two or three atoms.

As used herein, the abbreviations for any protective groups, amino acids and other compounds are, unless indicated otherwise in accord with their common usage, recognized abbreviations or the IUPAC-IUB Commission on Biochemical Nomenclature (Biochem., 1972, 11, 942-944).

As employed herein, the following terms have their accepted meaning in the chemical literature:

AcOH acetic acid

anhydr anhydrous

CDI 1,1′-carbonyldiimidazole

DCM dichloromethane

DIPA diisopropylamine

DIPEA diisoproylethylamine

DMAP 4-dimethylaminopyridine

DMDO dimethyldioxirane

DMF N,N-dimethylformamide

DMSO dimethyl sulfoxide

EDCI.HCl 1-ethyl-3-(3-dimethylaminopropyl)carbodiimide hydrochloride

Et₂O diethyl ether

EtOAc ethyl acetate

EtOH ethanol

HOBt 1-hydroxybenzotriazole

LG leaving group

MeOH methanol

MW microwave reactor

NMP N-methylpyrrolidine

NH₄Oac ammonium acetate

o.n. over night

Pd/C palladium on actived carbon

r.t. room temperature

TBAI tetrabutylammonium iodide

TEA triethylamine

Tfp tri-2-furylphosphine

THF tetrahydrofuran

When two substituents and the atoms to which they are attached form a ring, it is meant that that the two substituents are linked and that at least some of the atoms in the two substituents together with the atoms to which the substituents are attached make up the atoms in a ring. For example, for the following structure:

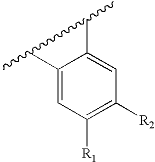

R₁ and R₂ may be linked to form a ring such as the following structure:

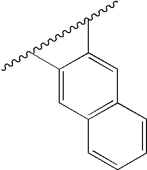

In the above example, R₁ and R₂ and the carbons to which they are attached form a six-membered aromatic ring.

When two substituents and the nitrogen to which they are attached form a fused heteroaryl, or heterocyclic ring, it is meant that the following structure:

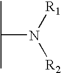

is representative of, for example, the following structures:

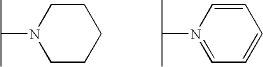

Unless otherwise indicated when a substituent is deemed to be “optionally substituted,” it is meant that the substitutent is a group that may be substituted with one or more group(s) individually and independently selected from alkyl, cycloalkyl, aryl, heteroaryl, heterocyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives thereof. The protecting groups that may form the protective derivatives of the above substituents are known to those of skill in the art and may be found in references such as Greene and Wuts, above.

If any compound described herein has one or more chiral centers, and an absolute stereochemistry is not expressly indicated, each center may independently be of R-configuration of S-configuration or a mixture thereof. Thus, the compounds provided herein may be enantiomerically pure or be stereoisomers of diastercomeric mixtures. In addition it is understood that, in any compound of this invention having one or more double bonds generating geometrical isomers that can be defined as E or Z, each double bond may independently be E or Z or a mixture thereof. Likewise, all tautomeric forms are also intended to be included.

Certain of the compounds disclosed herein may exist as stereoisomers including optical isomers. The scope of the present disclosure includes all stereoisomers and both the racemic mixtures of such stereoisomers as well as the individual enantiomers that may be separated according to methods that are well known to those of ordinary skill in the art.

The schemes, set forth below, provide examples of reaction schemes for the synthesis of the compounds of Formula I disclosed herein. For example compounds of Formula I may be synthesized according to the method depicted in Scheme 1.

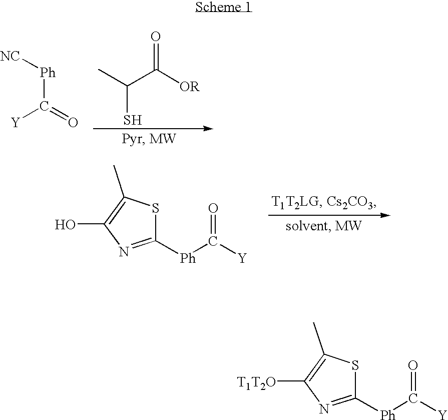

The condensation between a nitrile and a carboxylic acid derivative followed by an O-alkylation can provide compounds of Formula I. The condensation reaction is preferably carried out in a microwave reactor, preferably with a temperature about 150-180° C. and preferably between 5-15 min. The alkylation is preferably carried out in a microwave reactor, with a temperature about 150-180° C. and preferably between 15-25 min. In one embodiment, the reaction is carried out with acetonitrile as solvent. When necessary an in situ formation of T₁T₂I can be performed with e.g. NaI or KI. The final product is isolated by conventional means, and preferably purified by re-crystallization. Y, T₁ and T₂ have the definitions as described herein. R is defined as a branched or un-branched C₁-C₆ alkyl or haloalkyl, or phenyl optionally substituted. Ph is a phenyl, optionally additionally substituted at any of the open positions. LG is defined as a leaving group e.g. halide. Alternatively the compounds having general Formula I can be obtained according to the method depicted in Scheme 2.

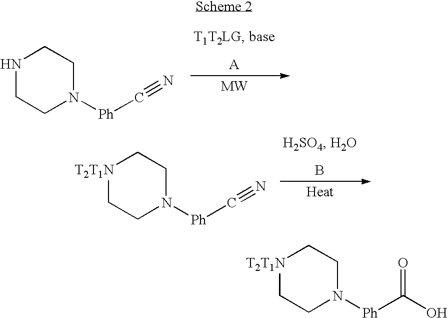

The alkylation of an amine followed by an acidic hydrolysis of a nitrile can provide compounds of Formula I. The alkylation is preferably carried out in a microwave reactor, with a temperature about 150-180° C., preferably at 160° C. and for about 5-15 min but preferably for 10 min. In one embodiment, the reaction is carried out with acetonitrile as a solvent and when needed, the reaction can be carried out with the presence of potassium iodide. The presence of a base is favored, preferably K₂CO₃. The hydrolysis of the nitrile is preferably carried out in a microwave reactor with a temperature about 100-130° C., preferably at 120° C. and for about 5-15 min but preferably for 5 min. T₁ and T₂ have the definitions as described above. Ph is a phenyl, optionally additionally substituted at any of the open positions. LG is defined as a leaving group, e.g. halide or another leaving group, that favors the reaction.

Alternatively the compounds having general Formula I can be obtained according to the method depicted in Scheme 3.

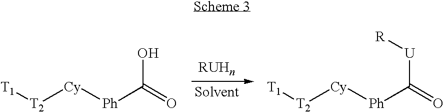

The acylation of a carboxylic acid can provide compounds of Formula I. The acylation is preferably carried out in the presence of coupling reagents, such as EDCI.HCl, and at a temperature about of 25-150° C., preferably at 25° C., and for about 5-24 h but preferably for 16 h. In one embodiment, the reaction is carried out with acetonitrile or DMF as solvent. A base is used when suitable, preferably DIPEA, TEA or DIPA. Cy, T₁, and T₂ have the definitions as described herein. n is an integer from 1 to 2. Ph is a phenyl, optionally additionally substituted at any of the open positions. RU may be defined as, but is not limited to, NR—NRR, OR and NR. However it is also possible to carry out the acylation reactions under conditions described by Green (373-451; Green et al., in Protective groups in organic synthesis; 3^{rd} edition; John Wiley & sons, Inc: New York, USA, 1999), which is incorporated herein by reference.

Alternatively the compounds having general Formula I can be obtained according to the method depicted in Scheme 4.

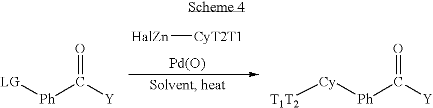

The sp²-sp³ cross coupling reaction between a cyclic electrophile and an aliphatic nucleophile or the sp²-sp² coupling between a cyclic electrophile and a cyclic nucleophile and can provide compounds of Formula I. The cross coupling reaction is carried out in the presence of a palladium catalyst, preferably Pd(PtBu₃)₂ or Pd₂(dba)₃ with tfp as a ligand. The reaction is preferably carried out with NMP/THF 1:2 as solvent. The temperature is about 25-100° C. and the reaction has gone to completion after 10 min-16 h. The product is isolated by conventional means and is preferably purified by flash chromatography. When LG is a triflate, the reaction is preferably carried out with the presence of TBAI. LG is defined as a leaving group e.g. halide, nonaflate or triflate. Cy, Y, T₁ and T₂ are defined as described herein. Ph is a phenyl, optionally additionally substituted at any of the open positions.

Alternatively the compounds having general Formula I can be obtained according to the method depicted in Scheme 5.

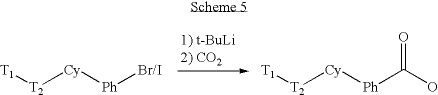

Compounds of Formula I where Y is —C(═O)OH, can be obtained through lithiation of bromides or iodides, at temperatures about −78° C.-20° C., but preferably at −20° C., in THF for 0.5-2 h, preferably 0.5 h, followed by addition of CO₂(g). The final product is obtained by conventional means and it is purified by use of an ion exchange column. Cy, T₁ and T₂ are defined as described herein. Ph is a phenyl, optionally additionally substituted at any of the open positions.

Alternatively the compounds having general Formula I can be obtained according to the method depicted in Scheme 6.

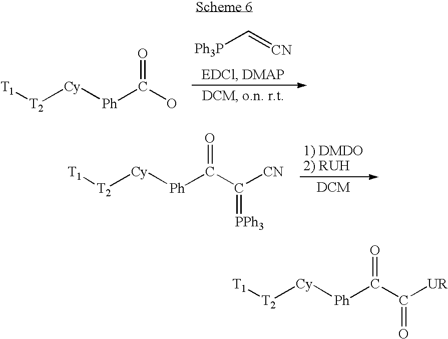

wherein Cy, T₁ and T₂ are defined as described herein. Ph is a phenyl, optionally additionally substituted at any of the open positions.

Alternatively the compounds having general Formula I can be obtained in two steps by first generating cyano keto phospheranes (Harry H. Wasserman, H. H., Hot, W-B.; J. Org. Chem., 1994, 59′ 4364-4366), then an oxidation by DMDO is performed (Wong, M-K. et al; J. Org. Chem., 2001, 66, 3606-3609) and the a keto acid is generated. Cy₁, Cy₂, T₁, T₂ and T₃ have the definitions as described above. RU can be defined as but is not limited to NR—NRR, OR and NR.

Alternatively the compounds having general Formula I can be obtained according to the method depicted in Scheme 7.

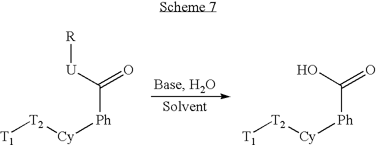

The hydrolysis of a carboxylic acid derivative can provide compounds of Formula I. The hydrolysis is preferably carried out in the presence of water and at a temperature about of 25-180° C., preferably at 160° C. and for a few minutes but preferably for 5 minutes when performed in a microwave reactor. However, the reaction can also be performed under traditional heating conditions, such as at reflux temperature. Preferably, the reaction is carried out with THF as solvent. A base is used when suitable, preferably, LiOH or NaOH. Cy, T₁ and T₂ are defined as described herein. Ph is a phenyl, optionally additionally substituted at any of the open positions. RU can be defined but is not limited to NR—NRR, OR and NR. However it is also possible to carry out the hydrolysis under conditions described by Green (Green et al. in Protective groups in organic synthesis; 3^{rd} edition; John Wiley & sons, Inc: New York, USA, 1999).

Alternatively the compounds having general Formula I can be obtained according to the method depicted in Scheme 8.

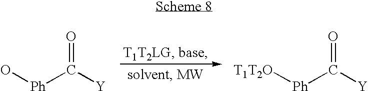

The O-alkylation can provide compounds of Formula I. The alkylation is preferably carried out in a microwave reactor, with a temperature about 150-180° C. and preferably between 15-25 min. In one embodiment, the reaction carried out with acetonitrile as solvent. The base is preferably Cs₂CO₃ but also other bases can be used as K₂CO₃. When necessary an in situ formation of T₁T₂I can be performed with e.g. NaI or KI. The final product is isolated by conventional means, and preferably purified by re-crystallization. Y, T₁ and T₂ have the definitions as described herein. R is defined as a branched or un-branched C₁-C₆ alkyl or halo-alkyl, or phenyl optionally substituted. Ph is a phenyl, optionally additionally substituted at any of the open positions. LG is defined as a leaving group e.g. halide.

In the context of the present disclosure, a “modulator” is defined as a compound that is an agonist, a partial agonist, an inverse agonist or an antagonist of one or more RARβ receptors. In the context of the present disclosure, an “agonist” is defined as a compound that increases the basal activity of a receptor (i.e. signal transduction mediated by the receptor). An “antagonist” is defined as a compound, which blocks the action of an agonist on a receptor. A “partial agonist” is defined as an agonist that displays limited, or less than complete, activity such that it fails to activate a receptor in vitro, functioning as an antagonist in vivo. An “inverse agonist” is defined as a compound that decreases the basal activity of a receptor.

The term “subject” refers to an animal, preferably a mammal, and most preferably a human, who is the object of treatment, observation or experiment. The mammal may be selected from the group consisting of mice, rats, rabbits, guinea pigs, dogs, cats, sheep, goats, cows, primates, such as monkeys, chimpanzees, and apes, and humans.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

The term “pharmaceutical composition” refers to a mixture of a compound disclosed herein with other chemical components, such as diluents or carriers. The pharmaceutical composition facilitates administration of the compound to a subject. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of a subject.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present disclosure thus may be formulated in a conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose about the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXAMPLES

The following examples are provided as an illustration of the present invention, but should in no way be considered as limiting the scope of invention itself.

### Example 1

### 2-Fluoro-4-(4-hydroxy-5-methyl-thiazol-2-yl)-benzoic acid ethyl ester (Scheme 1)

4-Cyano-2-fluoro-benzoic acid ethyl ester (386 mg, 2.0 mmol), 2-mercaptopropionic acid (178 μl, 2.0 mmol) and pyridine (15 μl, 1.0 mmol) were transferred to a MW-vial. The mixture was thoroughly mixed on a Whirl mixer and heated in the MW for 15 minutes at 150° C. this yielded a yellow solid. Pyridine was removed in vacuo. This procedure was repeated five times. The reaction mixtures were combined and wash with CH₃CN yielded 1.90 g (67%) of the title compound as a yellow solid. ¹H NMR (CDCl₃): δ 8.02-7.98 (m, 1H); 7.63-7.59 (m, 2H); 4.44 (q, J=7.04, 2H); 2.39 (s, 3H), 1.41 (t, J=7.03, 3H). ¹³C NMR (100 MHz, CDCl₃): δ 164.0; 164.0; 163.7; 161.1; 159.0; 157.6; 138.4; 138.3; 133.1; 132.9; 132.7; 121.0; 1209; 119.7; 119.6; 116.0; 114.1; 113.8; 110.5; 106.9; 61.7; 14.4; 9.6.

### Example 2

### 4-[4-(2-Butoxy-ethoxy)-5-methyl-triazol-2-yl]-2-fluoro-benzoic acid (Compound of Formula 3) (Scheme 1)

2-Fluoro-4-(4-hydroxy-5-methyl-thiazol-2-yl)-benzoic acid ethyl ester (281 mg, 1.0 mmol) was transferred to a MW-vial and added 2-butoxy ethyl bromide (362 mg, 2.0 mmol), Cs₂CO₃ (652 mg, 2.0 mmol) and CH₃CN (4 mL). The vial was heated in the MW for 25 minutes at 180° C. This procedure was repeated six times. The reaction mixtures were combined, filtered, concentrated in vacuo and the low boiling impurities were removed by Kugel-Rohr distillation (distillation stopped at 160° C., 5×10⁻² Torr). The resulting dark oil was divided into three aliquots and transferred to three MW-vials. Lithium hydroxide monohydrate (252 mg, 6.0 mmol.) and a 1:2 mixture of H₂O/THF (3 mL) were added to the vials. The vials were capped and heated to 160° C. for 5 minutes in the MW. The resulting mixtures were combined and transferred to a separation funnel with EtOAc. The organic phase was extracted with 2M NaOH and water. The water phase was acidified with 2M HCl and extracted with EtOAc. The organic phase was dried over Na₂SO₄, filtered and concentrated in vacuo to yield 1.45 g (68%) of the title compound as a yellow solid. ¹H NMR (CDCl₃): δ 8.04-8.00 (m, 1H); 7.66-7.61 (m, 2H); 4.53-4.51 (m, 2H); 3.80-3.78 (m, 2H); 3.57-3.53 (m, 2H); 2.33 (s, 3H); 1.62-1.56 (m, 2H); 1.41-1.30 (m, 2H); 0.92 (t, J=7.60, 3H). ¹³C NMR (CDCl₃): δ 168.6; 164.4; 161.8; 160.7; 160.6; 159.6; 156.0; 140.9; 140.8; 133.6; 120.7; 120.7; 117.7; 113.7; 113.4; 110.3; 71.5; 70.0; 69.7; 32.0; 19.5; 14.1; 9.7.

### Example 3

### 4-(5-Heptyl-pyrimidin-2-yl)-benzoic acid (Scheme 2B)

4-(5-Heptylpyrimidine-2-yl)benzonitrile (100 mg, 0.36 mmol) was mixed with water (0.4 mL), sulfuric acid (1.0 mL) and glacial acetic acid (1.0 mL). The mixture was heated to 120° C. and after 8 h the mixture was cooled to r.t. The reaction mixture was filtered and the solid was washed with 50% NaOH and then 4M HCl. Yield: 91 mg (85%). ¹³C NMR (100 MHz, DMSO): 167.0; 160.4; 157.3 (2C); 141.1; 134.1; 132.3; 129.7 (2C); 127.5 (2C); 31.2; 30.1; 29.2; 28.5; 28.4; 22.0; 13.9. LC/MS: Purity (UV/MS): 99/98.

### Example 4

### 4-Cyano-2-fluoro-benzoic acid ethyl ester (Scheme 3)

4-Cyano-2-fluorobenzoic acid (2.15 g, 13 mmol), abs. EtOH (1.59 mL, 27.3 mmol), 1-ethyl-3-(3-dimethylaminopropyl)carbodiimide hydrochloride (5.23 g, 27.3 mmol) and 1-hydroxybenzotriazole (3.69 g, 27.3 mmol) were transferred to a dry Schlenk flask and the flask was degassed and filled with argon. DMF (70 mL) was added and the mixture was cooled to 0° C. on an ice bath before DIPEA (3.9 mL, 27.3 mmol) was added. The reaction mixture was slowly warmed to r.t. and stirred for 14 hours. The reaction mixture was transferred to a separation funnel with EtOAc and washed with 5% citric acid, H₂O, 1M NaOH and brine. The organic phases were collected and dried over Na₂SO₄, filtered and concentrated in vacuo. Re-crystallisation with EtOAc and heptane yielded 2.34 g (93%) of the title compound as a white solid. ¹H NMR (CDCl₃): δ 8.07-8.03 (m, 1H); 7.53-7.51 (m, 1H); 7.47-7.44 (m, 1H); 4.44 (q, J=7.03, 2H); 1.41 (t, J=7.04, 3H). ¹³C NMR (100 MHz, CDCl₃): δ 163.1; 163.1; 162.7; 160.1; 133.3; 133.3; 127.9; 127.8; 123.9; 123.8; 121.2; 120.9; 117.6; 116.9; 116.9; 62.4; 14.4.

### Example 5

### 4′-Hydroxy-biphenyl-4-carboxylic acid ethyl ester (Scheme 3)

4′-Hydroxy-biphenyl-4-carboxylic acid (1071 mg, 5.0 mmol), abs. ethanol (3.0 mL) and cone. sulphuric acid (0.5 mL) were transferred to a 5 mL MW vial and the vial was capped. The mixture was heated to 170° C. for 1 min in a microwave reactor. After cool down the mixture was transferred with EtOAc to a separation funnel and washed with aqueous NaHCO₃ and brine. The organic phases were collected and dried over Na₂SO₄ and concentrated. Creamy white crystals (927 mg, 77% yield) of the title compound were obtained. ¹H NMR (400 MHz, CDCl₃): δ 8.12-8.07 (m, 2H); 7.63-7.58 (m, 2H), 7.54-7.50 (m, 2H); 6.97-6.92 (m, 2H); 4.41 (q, J=7.24, 2H); 1.41 (t, J=7.03, 3H). ¹³C NMR (100 MHz, CDCl₃): δ 166.9; 156.1, 145.3; 132.9; 130.3 (2C); 128.8 (2C); 126.7 (2C); 116.1 (2C); 61.2; 14.6.

### Example 6

### 4′-Nonyl-biphenyl-4-carboxylic acid ethyl ester (Scheme 4)

A Schlenk flask was dried and flushed with argon. Zinc dust (1059 mg, 16.2 mmol) was transferred to the flask where after the flask was evaporated again and filled with argon. 0.4 mL THF and 1,2-dibromoethane (46 μl, 101 mg, 0.54 mmol, 6 mol %) were added. The mixture was gently heated with a heat gun to boil the THF. After ca. 1 min the reaction mixture foamed and the heating was interrupted. After ca. 1 min the heating-cooling process was repeated two more times. 0.04 mL trimethylsilylchloride was added and the reaction mixture was stirred for 5 min. Then a solution of nonyl iodide (2287 mg, 9 mmol) in 3 mL THE with 3 drops of n-decane was slowly added. The mixture was then heated at r.t. for 1 h then at 50° C. for 2 h. The reaction mixture was checked by GC analysis and iodolysis. After completion of reaction the zinc suspension was allowed to settle. A Schlenk flask was dried and the tris(dibenzylideneacetone)depalladium(0) (115 mg, 0.125 mmol, 5 mol %), 2-(dicyclohexylphosphino)biphenyl (175 mg, 0.5 mmol, 20 mol %) and tetrabutylammonium-fluoride (924 mg, 2.5 mmol) was transferred to it. 2 mL NMP was added followed by 4′-trifluoromethanesulfonyloxy-biphenyl-4-carboxylic acid ethyl ester (935 mg, 2.5 mmol) and the previously made solution of the zinc reagent. The mixture was heated to 60° C. for 14 h. After cooling the mixture was quenched with aqueous NH₄Cl and an aqueous work-up with EtOAc was performed. The organic phases were combined and concentrated onto celite. The mixture was purified by flash chromatography (4 g column, 0-5% EtOAc in heptane) to give 744 mg (84% yield) of the title compound as white crystals.

¹H NMR (CDCl₃): δ 8.14-8.08 (m, 2H); 7.69-7.63 (m, 2H); 7.58-7.53 (m, 2H); 7.31-7.26 (m, 2H); 4.40 (q, J=7.03, 2H); 2.66 (t, J=7.63, 2H); 1.70-1.60 (m, 2H); 1.41 (t, J=7.04, 3H); 1.39-1.20 (m, 12H); 0.88 (t, J=7.63, 3H). ¹³C NMR (100 MHz, CDCl₃): δ 166.8; 145.7; 143.4; 137.6; 130.2 (2C); 129.2 (2C); 127.3 (2C); 127.0 (2C); 61.1; 35.9; 32.1; 31.6; 29.8; 29.7; 29.6; 29.5; 22.9; 14.6; 14.3.

### Example 7

### 4′-(trans-4-Pentyl-cyclohexyl)-biphenyl-4-carboxylic acid (Scheme 2)

Trans-4-cyano-4′-(4-N-pentylcyclohexyl)biphenyl (50 mg, 0.16 mmol) was mixed with water (0.2 mL), sulfuric acid (0.5 mL) and glacial acetic acid (0.5 mL). The mixture was heated to 120° C. and after 8 h the mixture was cooled to r.t. The reaction mixture was filtered and the solid was washed with 50% NaOH and then 4M HCl. Yield: 51 mg (91%). LC/MS: Purity (UV/MS): 85/90.

### Example 8

### 4′-Nonyl-biphenyl-4-carboxylic acid (Compound of Formula 2) (Scheme 8)

352 mg (1 mmol) 4′-nonyl-biphenyl-4-carboxylic acid ethyl ester and 126 mg (3 mmol) lithium hydroxide monohydrate was placed in a 5 mL microwave vial and was added a 1:2 mixture of H₂O/THF. The mixture was capped and heated to 160° C. for 5 min. The reaction mixture was washed with water and the org. phases were combined, dried over Na₂SO₄, filtered and concentrated in vacuo yielding 221 mg (68%) of the title compound as white crystals. ¹H NMR (CDCl₃): δ 8.22-8.14 (m, 2H); 7.72-7.67 (m, 2H); 7.60-7.54 (m, 2H); 7.32-7.27 (m, 2H); 2.66 (t, J=7.60, 2H); 1.72-1.60 (m, 2H); 1.43-1.22 (m, 12H); 0.88 (t, J=6.80, 3H). ¹³C NMR (CDCl₃): δ 171.0; 143.6; 137.4; 130.9 (2C); 129.3 (2C); 127.8; 127.4 (2C); 127.1 (2C); 35.9; 32.1; 31.6; 29.8; 29.7; 29.5; 29.5; 22.9; 14.3.

### Example 9

### General Procedure 1 (GP1) (Based on General Scheme 3)

### 4′-Octyl-biphenyl-4-carboxylic acid furan-2-ylmethyl ester

PS-Triphenylphospine (3 mmol PPh₃/resin) (167 mg, 0.5 mmol) was added carbon tetrachloride (1 mL) and DCM (3 mL) followed by 4-octylbiphenyl-4′-carboxylicacid (78 mg, 0.25 mmol) and the reaction mixture was heated to 80° C. for 22 h. The mixture was cooled to 50° C. and furfuryl alcohol (0.02 mL, 0.23 mmol) and N-methyl morpholine (0.03 mL, 0.27 mmol) were added and the mixture was heated at 50° C. for 64 h. Then the mixture was cooled to r.t. and filtered through a glass funnel. Half of the reaction mixture was poured into a mixture of DCM (4 mL) and PS-trisamine (106 mg, ˜0.3 mmol). The mixture was stirred at r.t. for 16 h. The mixture was filtered and the filtrate conc. in vacuo. Yield: 18 mg (40%). ¹H NMR (CDCl₃): δ 8.12-8.07 (m, 2H); 7.67-7.61 (m, 21); 7.56-7.51 (m, 2H); 7.47-7.44 (m, 1H); 7.40-7.24 (m, 2H); 6.51-6.48 (m, 1H); 6.41-6.38 (m, 1H), 5.33 (s, 2H); 2.65 (q, J=7.60, 2H); 1.70-1.59 (m, 2H); 1.42-1.20 (m, 12H); 0.89 (t, J=6.80, 3H). ¹³C NMR (100 MHz, CDCl₃): δ 166.4; 149.9; 146.1; 143.5; 143.5; 137.5; 130.5 (2C); 129.3 (2C); 128.6; 127.4 (2C); 127.0 (2C); 111.0; 110.8; 58.7; 35.9; 32.1; 31.7; 30.3; 29.7; 29.6; 29.5; 22.9; 14.3.

### 4′-Octyl-biphenyl-4-carboxylic acid phenethyl-amide

PS-Triphenylphospine (3 mmol PPh₃/resin) (183 mg, 0.55 mmol) was added carbon tetrachloride (2 mL) and DCM (3 mL) followed by 4-octylbiphenyl-4′-carboxylicacid (77 mg, 0.25 mmol). Finally phenethylamine (0.03 mL, 0.25 mmol) and N-methyl morpholine (0.03 mL, 0.27 mmol) were added and the mixture was heated at 50° C. for 64 h. Then the mixture was cooled to r.t. and filtered through a glass funnel, followed by wash of the collected resin with DCM. The combined filtrate and washed was washed with 0.1M HCl, brine, NaHCO₃ (sat.) and brine. The organic layer was dried over Na₂SO₄, filtered and conc. in vacuo. Yield: 74 mg (71%). ¹H NMR (CDCl₃): δ 7.76-7.72 (m, 2H); 7.64-7.59 (m, 2H); 7.53-7.49 (m, 2H); 7.35-7.31 (m, 2H); 7.29-7.23 (m, 5H); 6.13 (s, 1H); 3.75 (q, J=0.83, 2H); 2.96 (t, J=6.84, 2H); 2.65 (t, J=7.81, 2H); 1.70-1.60 (m, 2H); 1.42-1.20 (m, 10H); 0.88 (t, J=6.20, 3H). ¹³C NMR (100 MHz, CDCl₃): δ 166.2; 143.2; 142.0, 138.0; 136.3; 132.0; 128.0 (2C); 127.8 (2C); 127.7 (2C); 126.3 (2C); 126.0 (2C); 125.6; 40.1; 34.8; 34.6; 30.9; 30.4; 28.5; 28.3; 28.2; 21.7; 13.1.

### Example 10

### General Procedure 2 (GP2) (Based on General Scheme 3)

### 4′-Octyl-biphenyl-4-carboxylic acid benzylamide

PS-Triphenylphospine (3 mmol PPh₃/resin) (184 mg, 0.55 mmol) was added carbon tetrachloride (2 mL) and DCM (3 mL) followed by 4-octylbiphenyl-4′-carboxylicacid (93 mg, 0.30 mmol). Finally benzylamine (0.03 mL, 0.25 mmol) and N-methyl morpholine (0.04 mL, 0.27 mmol) were added and the mixture was heated at 50° C. for 64 h. Then the mixture was cooled to r.t. and filtered through a glass funnel, followed by wash of the collected resin with a small amount of DCM. The combined wash and filtrate was added PS-trisamine (50 mg, ˜0.3 mmol). The mixture was stirred at r.t. for 16 h. The mixture was filtered and the filtrate conc. in vacuo. Yield: 123 mg (quan. yield). ¹H NMR (DMSO): δ 9.19-9.20 (m, 1H); 8.00-7.95 (m, 2H); 7.78-7.73 (m, 2H); 7.67-7.61 (m, 2H); 7.37-7.18 (m, 7H); 4.53-4.48 (m, 2H); 2.66-2.58 (m, 2H), 1.66-1.55 (m, 2H); 1.46-1.18 (m, 10H); 0.86 (t, J=6.65, 3H).

### 4-Octyl-biphenyl-4-carboxylic acid (2-cyano-ethyl)-amide

The title compound was prepared according to GP2 from 3-aminopropionitrile (19 mg, 0.27 mmol) and 4-octylbiphenyl-4′-carboxylicacid (93 mg, 0.30 mmol), Yield: 49 mg (50%). ¹H NMR (CDCl₃): δ 7.88-7.83 (m, 2H); 7.68-7.63 (m, 2H); 7.56-7.50 (m, 2H); 7.30-7.25 (m, 2H); 6.82 (s, 2H); 3.73 (q, J=6.25, 2H); 2.76 (t, J=6.25, 2H); 2.65 (t, J=7.62, 2H); 1.73-1.60 (m, 2H); 1.40-1.20 (m, 10H); 0.89 (t, J=6.45, 3H). ¹³C NMR (100 MHz, CDCl₃): δ 167.9; 145.0; 143.3; 137.2; 132.0; 129.2 (2C); 127.7 (2C); 127.3 (2C); 127.2 (2C); 118.4; 36.4; 35.8; 32.0; 31.6; 29.6; 29.5; 29.4; 22.8; 18.7; 14.2.

### 4′-Octyl-biphenyl-4-carboxylic acid furan-2-ylmethyl ester

The title compound was prepared according to GP2 from furfurylamine (26 mg, 0.27 mmol) and 4-octylbiphenyl-4′-carboxylicacid (93 mg, 0.30 mmol). Yield: 66 mg (50%). LC/MS: Purity (UV/MS): 36/-.

### Example 11

### General Procedure 3 (GP3) (Based on General Scheme 3)

### 4′-Hexyloxy-biphenyl-4-carboxylic acid (2-pyridin-2-yl-ethyl)-amide

PS-Triphenylphospine (3 mmol PPh₃/resin) (230 mg, 0.69 mmol) was added carbon tetrachloride (2 mL) and DCM (3 mL) followed by 4′-hexyloxybiphenyl-4′-carboxylic acid (104 mg, 0.35 mmol). Finally, 2-2-aminoethylpyridine (0.04 mL, 43 mg, 0.35 mmol) and N-methyl morpholine (0.05 mL, 0.42 mmol) were added and the mixture was heated at 50° C. for 64 h. The crude mixture was run through a PSA and a SCX ion-exchange column. The filtrate was conc. in vacuo. Yield: 100 mg (69%). ¹H NMR: Purity: >90%. ¹H NMR (CDCl₃): δ 8.60-8.56 (m, 1H); 7.84-7.80 (m, 2H); 7.67-7.57 (m, 3H); 7.57-7.50 (m, 2H); 7.24-7.15 (m, 2H); 7.00-6.95 (m, 2H); 4.00 (t, J=6.64, 2H); 3.90 (q, J=5.67, 2H); 3.12 (t, J=5.86, 2H); 1.85-1.70 (m, 2H), 1.50-1.25 (m, 8); 0.90 (t, J=6.84, 3H). ¹³C NMR (100 MHz, CDCl₃): δ 160.1; 149.3; 143.9; 136.9; 128.4 (2C); 127.6 (2C); 127.7 (2C); 123.7; 121.8; 115.1 (2C); 68.3; 39.3; 36.8; 31.9; 29.4; 29.2; 26.2; 22.6; 14.2.

### 4′-Hexyloxy-biphenyl-4-carboxylic acid (2-cyano-ethyl)-amide

The title compound was prepared according to GP3 from 3-aminopropionitrile (25 mg, 0.35 mmol) and 4′-hexyloxybiphenyl-4′-carboxylic acid (104 mg, 0.35 mmol). Yield: 75 mg (61%). ¹H NMR: Purity: >90%.

### 4′-Heptyloxy-biphenyl-4-carboxylic acid (2-cyano-ethyl)-amide

The title compound was prepared according to GP3 from 3-aminopropionitrile (25 mg, 0.35 mmol) and 4′-heptyloxybiphenyl-4′-carboxylicacid (109 mg, 0.35 mmol). Yield: 59 mg (46%). LC/MS:

### 4′-Hepyyloxy-biphenyl-4-carboxylic Acid (furan-2-ylmethyl)-amide

The title compound was prepared according to GP3 from 4-furfurylamine (0.03 mL, 33 mg, 0.35 mmol) and 4′-heptyloxybiphenyl-4′-carboxylicacid (109 mg, 0.35 mmol). Yield: 116 mg (85%). Purity (UV/MS): 76/-.

### 4′-Octyloxy-biphenyl-4-carboxylic acid (2-pyridin-2-yl-ethyl)-amide (Compound of Formula 54)

The title compound was prepared according to GP3 from 2-2-aminoethylpyridine (0.04 mL, 43 mg, 0.35 mmol) and 4′-octyloxybiphenyl-4′-carboxylicacid (114 mg, 0.35 mmol). Yield: 146 mg (97%).

### 4′-Octyloxy-biphenyl-4-carboxylic acid (2-cyano-ethyl)-amide (Compound of Formula 51)

The title compound was prepared according to GP3 from 3-aminopropionitrile (25 mg, 0.35 mmol) and 4′-octyloxybiphenyl-4′-carboxylicacid (114 mg, 0.35 mmol). Yield: 70 mg (53%).

### 4′-Octyloxy-biphenyl-4-carboxylic acid (furan-2-ylmethyl)-amide

The title compound was prepared according to GP3 from 4-furfurylamine ((0.03 mL, 33 mg, 0.35 mmol) and 4′-octyloxybiphenyl-4′-carboxylicacid (114 mg, 0.35 mmol). Yield: 121 mg (85%). Purity (UV/MS): 73/90.

### Example 12

### General Procedure 4 (GP4) (Based on General Scheme 3)

### 4′-Hexyl-biphenyl-4-carboxylic acid (2-pyridin-2-yl-ethyl)-amide

PS-Triphenylphospine (3 mmol PPh₃/resin) (161 mg, 0.48 mmol) was added carbon tetrachloride (2 mL) and DCM (3 mL) followed by the 4′-hexylbiphenyl carboxylic acid (50 mg, 0.15 mmol). Finally, 2-(2′-aminoethyl)pyridine (22 mg, 0.18 mmol) and N-methyl morpholine (0.05 mL, 0.42 mmol) were added and the mixture was heated to 50° C. for 64 h. The crude mixture was ran through a PSA ion-exchange column and a SCX ion exchange. The filtrate was conc. in vacuo. The crude solid was added 4 mL DCM and PS-trisamine (50 mg, ˜0.3 mmol). The mixture was stirred at r.t. for 16 h. The mixture was filtered and run through another PSA ion-exchange column and the subsequent filtrate conc. in vacuo Yield: 4 mg (6%). ¹H NMR (MeOD): δ 8.51-8.47 (m, 1H); 7.84-7.74 (m, 3H); 7.67-7.63 (m, 2H); 7.56-7.51 (m, 2H); 7.40-7.36 (m, 1H); 7.31-7.22 (m, 3H); 3.75 (t, J=7.03, 2H); 3.12 (t, J=7.23, 2H); 2.63 (t, J=7.62, 2H); 1.68-1.58 (m, 2H), 1.40-1.25 (m, 6H); 0.89 (t, J=6.65, 3H). ¹³C NMR (100 MHz, CDCl₃): δ 169.9; 160.2; 149.4; 145.6; 144.1; 139.2; 138.5; 134.0; 130.1 (2C); 128.8 (2C); 128.0 (2C); 127.8; 125.4; 123.4; 40.9; 38.2; 36.5; 32.9; 32.6; 30.0; 23.6; 14.4. LC/MS: Purity (UV/MS): 94/68.

### 4-Hexyl-biphenyl-4-carboxylic acid (furan-2-ylmethyl)-amide

The title compound was prepared according to GP4 from 4-furfurylamine (17 mg, 0.18 mmol) and 4′-hexylbiphenyl-4′-carboxylicacid (50 mg, 0.35 mmol). Yield: 4 mg (6%). LC/MS: Purity (UV/MS): 100/100.

### 4′-Hexyl-biphenyl-4-carboxylic acid (5-methyl-pyridin-2-yl)-amide (Compound of Formula 63) (71BG53-1D)

The title compound was prepared according to GP4 from 2-amino-5-methylpyridine (19 mg, 0.18 mmol) and 4′-hexylbiphenyl-4′-carboxylicacid (50 mg, 0.35 mmol). Yield: 33 mg (51%). LC/MS: Purity (UV/MS): 90/97.

### 4′-Heptyl-biphenyl-4-carboxylic acid (2-pyridin-2-yl-ethyl)-amide (Compound of Formula 59)

The title compound was prepared according to GP4 from 2-(2′-aminoethyl)pyridine (22 mg, 0.18 mmol) and 4′-heptylbiphenyl-4′-carboxylicacid (52 mg, 0.35 mmol). Yield: 30 mg (42%). LC/MS: Purity (UV/MS): 90/81.

### 4′-Heptyl-biphenyl-4-carboxylic acid (2-cyano-ethyl)-amide

The title compound was prepared according to GP4 from 3-aminopropionitrile (13 mg, 0.18 mmol) and 4′-heptylbiphenyl-4′-carboxylicacid (52 mg, 0.35 mmol). Yield: 6 mg (10%). LC/MS: Purity (UV/MS): 99/99.

### 4′-Heptyl-biphenyl-4-carboxylic Acid (furan-2-ylmethyl)-amide

The title compound was prepared according to GP4 from furfurylamine (17 mg, 0.18 mmol) and 4′-heptylbiphenyl-4′-carboxylicacid (52 mg, 0.35 mmol). Yield: 19 mg (28%). LC/MS: Purity (UV/MS): 100/100.

### 4′-Heptyl-biphenyl-4-carboxylic acid (5-methyl-pyridin-2-yl)-amide

The title compound was prepared according to GP4 from 2-amino-5-methylpyridine (19 mg, 0.18 mmol) and 4′-heptylbiphenyl-4′-carboxylicacid (52 mg, 0.35 mmol). Yield: 17 mg (25%). LC/MS: Purity (UV/MS): 98/100.

### Example 13

### General Procedure 5 (GP5) (Based on General Scheme 3)

### 4′-Octyl-biphenyl-4-carboxylic acid furan-2-ylmethyl ester (Compound of Formula 48)

4-Octyl-biphenyl-4′-carboxylic acid (200 mg, 0.64 mmol) was in a 4 mL screw cap vial and thionyl chloride (1.0 mL) was carefully added and the mixture was heated to 79° C. After 2 h the mixture was concentrated in vacuo. The mixture was dissolved in 2.0 mL pyridine. The mixture was divided into two aliquots (1.1 mL each).

Furfuryl alcohol (22 μl, 0.26 mmol) was transferred to another 4 mL screw cap vial and the vial was flushed with argon. One of the pyridine aliquots (0.55 mL) was added to the alcohol. The vial was capped and heated to 90° C. for 15 h. The reaction mixture was cooled to r.t., run through a PSA ion exchange column and afterward evaporated in vacuo. Yield: 95 mg (quantitative yield). ¹H NMR (400 MHz, CDCl₃: 8.20-8.08 (m, 2H), 7.66-7.61 (m, 2H), 7.56-7.51 (m, 2H), 7.46-7.44 (m, 1H), 7.29-7.24 (m, 2H), 6.52-6.48 (m, 1H), 6.41-6.37 (m, 1H), 5.35 (s, 2H), 2.63 (t, J=7.63, 2H), 1.75-1.50 (m, 2H), 1.45-1.10 (m, 10H), 0.88 (t, J=7.63, 3H). ¹³C NMR (100 MHz, CDCl₃): 166.4; 149.9; 146.0; 143.5; 143.4; 137.4; 130.4 (2C); 129.2 (2C); 128.5; 127.3 (2C); 127.0 (2C); 110.9; 110.8; 58.7; 35.8; 32.1; 31.6; 29.6; 29.5; 29.4; 22.8; 14.3. LCMS: Purity (UV/MS): 97/-.

### 4′-Octyl-biphenyl-4-carboxylic acid 2-cyano-ethyl ester (Compound of Formula 34)

The title compound was prepared according to GP5 from 3-hydroxy propionitrile (18 μl, 0.26 mmol) and 4′-octylbiphenyl-4-carboxylic acid (200 mg, 0.64 mmol). Yield: 92 mg (94%). ¹H NMR (400 MHz, CDCl3): 8.20-8.08 (m, 2H), 7.66-7.61 (m, 2H), 7.56-7.51 (m, 2H), 7.29-7.24 (m, 2H), 4.58 (t, J=7.63, 2H), 2.95 (t, J=7.63, 2H), 2.63 (t, J=7.63, 2H), 1.75-1.60 (m, 2H), 1.45-1.20 (m, 10H), 0.88 (t, J=7.63, 3H). ¹³C NMR (100 MHz, CDCl₃): 166.1; 146.5; 143.6; 137.3; 130.5 (2C); 129.3 (2C); 127.7 (2C); 127.3 (2C); 117.0; 59.3; 35.9; 32.1; 31.6; 29.7; 29.6; 29.5; 22.9; 18.4; 14.3.

### 4′-Octyl-biphenyl-4-carboxylic acid (4-hydroxy-phenyl)-amide

The title compound was prepared according to GP5 from 4-Aminophenol (16 mg, 0.14 mmol) and 4′-octylbiphenyl-4-carboxylic acid (100 mg, 0.32 mmol). Yield: 27 mg (48%). ¹H NMR (Pyridine): δ 8.00-7.85 (m, 2H); 7.62-7.58 (m, 2H); 7.58-7.52 (m, 2H); 7.46-7.44 (m, 1H); 7.05-6.95 (m, 2H); 6.51-6.48 (m, 1H); 6.41-6.38 (m, 1H), 5.35 (s, 2H); 4.00 (q, J=7.03, 2H); 1.85-1.55 (m, 2H); 1.55-1.42 (m, 2H); 1.42-1.20 (m, 8H); 0.88 (t, J=7.63, 3H). LC/MS: Purity (UV/MS): 99/100.

### Example 14

### General Procedure 6 (GP6) (Based on General Scheme 3)

### 4′-Hexyl-biphenyl-4-carbolic acid 2-cyano-ethyl ester

4-Hexyl-biphenyl-4′-carboxylic acid (235 mg, 0.83 mmol) was in a 4 mL screw cap vial and thionyl chloride (1.5 mL) was carefully added and the mixture was heated to 79° C. After 2 h the mixture was concentrated in vacuo. The mixture was dissolved in 2.0 mL pyridine. The mixture was divided into four aliquots (0.55 mL each). 3-Hydroxy propionitrile (13 μl, 0.19 mmol) was transferred to another 4 mL screw cap vial and the vial was flushed with argon. One of the pyridine aliquots (0.55 mL) was added to the alcohol. The vial was capped and heated to 90° C. for 15 h. The reaction mixture was cooled to r.t., run through a PSA ion exchange column and afterward evaporated in vacuo. Yield: 23 mg (37%). ¹H NMR (400 MHz, CDCl₃): 8.20-8.08 (m, 2H), 7.70-7.65 (m, 2H), 7.58-7.55 (m, 2H), 7.29-7.24 (m, 2H), 4.58 (t, J=7.63, 2H), 2.95 (t, J=7.63, 2H), 2.63 (t, J=7.63, 2H), 1.75-1.60 (m, 2H), 1.45-1.20 (m, 10H), 0.88 (t, J=7.63, 3H). LC/MS: purity (UV/MS): 55/98.

### 4′-Heptyl-biphenyl-4-carboxylic acid 2-cyano-ethyl ester (Compound of Formula 19)

The title compound was prepared according to GP6 from 3-hydroxy propionitrile (13 μL, 13 mg, 0.19 mmol) and 4′-heptylbiphenyl-4′-carboxylic acid (247 mg, 0.21 mmol). Yield: 29 mg (45%). LC/MS: purity (UV/MS): 94/98.

### 4′-Heptyloxy-biphenyl-4-carboxylic acid 2-cyano-ethyl ester (Compound of Formula 23)

The title compound was prepared according to GP6 from 3-hydroxy propionitrile (13 μL, 13 mg, 0.19 mmol) and 4′-heptyloxy-biphenyl-4′-carboxylic acid (260 mg, 0.21 mmol). Yield: 42 mg (62%). LC/MS: purity (UV/MS): 92/100.

### 4′-Octyloxy-biphenyl-4-carboxylic acid 2-cyano-ethyl ester (Compound of Formula 42)

The title compound was prepared according to GP6 from 3-hydroxy propionitrile (13 μL, 13 mg, 0.19 mmol) and 4′-octyloxy-biphenyl-4′-carboxylic acid (272 mg, 0.21 mmol). Yield: 47 mg (67%).

¹H NMR (400 MHz, CDCl₃): 8.15-8.05 (m, 2H), 7.66-7.61 (m, 2H), 7.60-7.55 (m, 2H), 7.00-6.90 (m, 2H), 4.58 (t, J=7.63, 2H), 4.05 (t, J=7.63, 2H), 2.85 (t, J=7.63, 2H), 2.63 (t, J=7.63, 2H), 1.85-1.65 (m, 2H), 1.55-1.20 (m, 10H), 0.88 (t, J=7.63, 3H). ¹³C NMR (100 MHz, CDCl₃): 166.1; 159.8; 146.2; 132.1; 130.5 (2C); 128.6 (2C); 127.3 (2C); 127.8 (2C); 117.0; 115.2; 68.4; 59.3; 32.0; 29.6; 29.5; 29.4; 26.3; 22.9; 18.4; 14.3. LC/MS: purity (UV/MS): 91/100.

### 4′-Hexyl-biphenyl-4-carboxylic acid furan-2-ylmethyl ester

The title compound was prepared according to GP6 from furfuryl alcohol (16 μL, 18 mg, 0.19 mmol) and 4′-hexyl-biphenyl-4′-carboxylic acid (235 mg, 0.21 mmol). Yield: 37 mg (55%). LC/MS: purity (UV/MS): 52/97.

### 4′-Heptyl-biphenyl-4-carboxylic acid furan-2-ylmethyl Ester (Compound of Formula 17)

The title compound was prepared according to GP6 from furfuryl alcohol (16 μL, 18 mg, 0.19 mmol) and 4′-heptylbiphenyl-4′-carboxylic acid (247 mg, 0.21 mmol). Yield: 42 mg (60%). ¹H NMR (400 MHz, CDCl₃): 8.13-8.09 (m, 2H), 7.67-7.62 (m, 2H), 7.56-7.51 (m, 2H), 7.47-7.44 (m, 1H), 7.30-7.24 (m, 2H), 6.52-6.49 (m, 1H), 6.41-6.38 (m, 1H), 5.35 (s, 2H), 2.65 (t, J=7.63, 2H), 1.75-1.60 (m, 2H) (1.40-1.20 (m, 8H) (0.88 (t, J=7.63, 3H).

### 4′-Hexyloxy-biphenyl-4-carboxylic acid furan-2-ylmethyl ester (Compound of Formula 41)

The title compound was prepared according to GP6 from furfuryl alcohol (16 μL, 18 mg, 0.19 mmol) and 4′-hexyloxy-biphenyl-4-carboxylic acid (249 mg, 0.21 mmol). Yield: 67 mg (96%). LC/MS: purity (UV/MS): 97/99.

### 4′-Heptyloxy-biphenyl-4-carboxylic acid 2-cyano-ethyl ester (Compound of Formula 39)

The title compound was prepared according to GP6 from 3-hydroxy propionitrile (13 μL, 13 mg, 0.19 mmol) and 4′-heptyloxy-biphenyl-4-carboxylic acid (260 mg, 0.21 mmol). Yield: 47 mg (64%). ¹H NMR (400 MHz, CDCl₃)-8.15-8.05 (m, 2H), 7.70-7.65 (m, 2H), 7.65-7.50 (m, 2H), 7.45-7.40 (m, 1H), 7.00-6.90 (m, 2H), 6.50-6.45 (m, 1H), 6.40-6.35 (m, 1H), 5.35 (s, 2H), 3.95 (t, J=7.63, 2H), 1.85-1.75 (m, 2H), 1.55-1.20 (m, 8H), 0.88 (t, J=7.63, 3H).

### 4′-Octyloxy-biphenyl-4-carboxylic acid furan-2-ylmethyl ester (Compound of Formula 43)

The title compound was prepared according to GP6 from furfuryl alcohol (16 μL, 18 mg, 0.19 mmol) and 4′-octyloxy-biphenyl-4-carboxylic acid (272 mg, 0.21 mmol). Yield: 31 mg (41%). ¹H NMR (400 MHz, CDCl₃): 8.12-8.10 (m, 2H), 7.64-7.58 (m, 2H), 7.58-7.52 (m, 2H), 7.48-7.42 (m, 1H), 7.03-6.95 (m, 2H), 6.53-6.47 (m, 1H), 6.42-6.36 (m, 1H), 5.33 (s, 2H), 4.00 (t, J=6.63, 2H), 1.87-1.75 (m, 2H), 1.56-1.23 (m, 10H), 0.90 (t, J=6.14, 3H).

### 4′-Heptyloxy-biphenyl-4-carboxylic acid 2-pyridin-2-yl-ethyl ester (Compound of Formula 30)

The title compound was prepared according to GP6 from 2-(2-hydroxyethyl)pyridine (21 μL, 23 mg, 0.19 mmol) and 4′-heptyloxy-biphenyl-4-carboxylic acid (260 mg, 0.21 mmol). Yield: 13 mg (17%). LC/MS: purity (UV/MS): 13/58.

### 4′-Octyloxy-biphenyl-4-carboxylic acid furan-2-ylmethyl ester

The title compound was prepared according to GP6 from furfuryl alcohol (16 μL, 18 mg, 0.19 mmol) and 4′-octyloxy-biphenyl-4′-carboxylic acid (272 mg, 0.21 mmol). Yield: 31 mg (41%). ¹H NMR (CDCl₃): δ 8.12-8.06 (m, 2H); 7.62-7.58 (m, 2H); 7.58-7.52 (m, 2H); 7.46-7.44 (m, 1H); 7.05-6.95 (m, 2H); 6.51-6.48 (m, 1H); 6.41-6.38 (m, 1H), 5.35 (s, 2H); 4.00 (q, J=7.03, 2H); 1.85-1.55 (m, 2H); 1.55-1.42 (m, 2H); 1.42-1.20 (m, 8H); 0.88 (t, J=7.63, 3H).

### 4′-Hexyl-biphenyl-4-carboxylic acid (4-hydroxy-phenyl)-amide

The title compound was prepared according to GP6 from 4-aminophenol (21 mg, 0.19 mmol) and 4′-hexyl-biphenyl-4-carboxylic acid (235 mg, 0.21 mmol). Yield: 33 mg (48%). ¹H NMR (DMSO): δ 8.00-7.85 (m, 2H); 7.62-7.58 (m, 2H); 7.58-7.52 (m, 2H); 7.46-7.44 (m, 1H); 7.05-6.95 (m, 2H); 6.51-6.48 (m, 1H); 6.41-6.38 (m, 1H), 5.35 (s, 2H); 4.00 (q, J=7.03, 2H); 1.85-1.55 (m, 2H); 1.55-1.42 (m, 2H); 1.42-1.20 (m, 8H); 0.88 (t, J=7.63, 3H). LC/MS: Purity (UV/MS): 95/-.

### 4′-Heptyl-biphenyl-4-carboxylic acid (4-hydroxy-phenyl)-amide

The title compound was prepared according to GP6 from 4-aminophenol (21 mg, 0.19 mmol) and 4′-heptylbiphenyl-4′-carboxylic acid (247 mg, 0.21 mmol). Yield: 50 mg (70%). LC/MS: Purity (UV/MS): 95/99.

### 4′-Hexyloxy-biphenyl-4-carboxylic acid (4-hydroxy-phenyl)-amide

The title compound was prepared according to GP6 from 4-aminophenol (21 mg, 0.19 mmol) and 4′-hexyloxy-biphenyl-4-carboxylic acid (249 mg, 0.21 mmol). Yield: 53 mg (72%). LC/MS: Purity (UV/MS): 97/68.

### 4′-Heptyloxy-biphenyl-4-carboxylic acid (4-hydroxy-phenyl)-amide

The title compound was prepared according to GP6 from 4-aminophenol (21 mg, 0.19 mmol) and 4′-heptyloxy-biphenyl-4-carboxylic acid (260 mg, 0.21 mmol). Yield: 45 mg (60%). ¹H NMR (Pyridine): δ 8.00-7.85 (m, 2H); 7.62-7.58 (m, 2H); 7.58-7.52 (m, 2H); 7.46-7.44 (m, 1H); 7.05-6.95 (m, 2H); 6.51-6.48 (m, 1H); 6.41-6.38 (m, 1H), 5.35 (s, 2H); 4.00 (q, J=7.03, 2H); 1.85-1.55 (m, 2H); 1.55-1.42 (m, 2H); 1.42-1.20 (m, 5H); 0.88 (t, J=7.63, 3H). LC/MS: Purity (UV/MS): 100/-.

### 4′-Octyloxy-biphenyl-4-carboxylic acid (4-hydroxy-phenyl)-amide

The title compound was prepared according to GP6 from 4-aminophenol (21 mg, 0.19 mmol) and 4′-octyloxy-biphenyl-4′-carboxylic acid (272 mg, 0.21 mmol). Yield: 58 mg (73%). ¹H NMR (Pyridine): δ 8.00-7.85 (m, 2H); 7.62-7.58 (m, 2H); 7.58-7.52 (m, 2H); 7.46-7.44 (m, 1H); 7.05-6.95 (m, 2H); 6.51-6.48 (m, 1H); 6.41-6.38 (m, 1H), 5.35 (s, 2H); 4.00 (q, J=7.03, 2H); 1.85-1.55 (m, 2H); 1.55-1.42 (m, 2H); 1.42-1.20 (m, 8H); 0.88 (t, J=7.63, 3H). LC/MS: Purity UV/MS): 92/-.

### Example 15

### General Procedure 7 (GP7) (Based on General Scheme 8)

### 4′-[2-(Hexylamino)-2-oxoethoxy][1,1′-biphenyl]-4-carboxylic acid

4′-Hydroxy-biphenyl-4-carboxylic acid ethyl ester (48 mg, 0.2 mmol), 2-chloro-N-hexylacetamide (71 mg, 0.2 mmol), potassium carbonate (60 mg, 0.4 mmol), potassium iodide (35 mg, 0.2 mmol) and acetonitrile (1 mL) was transferred to a 0.5-2.0 mL MW vial and was heated to 180° C. for 25 min. The mixture was purified by combiflash (5:1 Hep/EtOAc). ¹H NMR (Pyridine): δ 8.51 (s, 1H); 8.27-8.21 (m, 2H); 7.75-7.71 (m, 2H); 7.67-7.62 (m, 2H); 7.15-7.09 (m, 2H); 4.84 (s, 2H); 4 35 (t, J=6.80, 2H); 3 48 (t, J=6.80, 2H); 1.61-1.52 (m, 2H); 1.39-1.12 (m, 9H); 0.79 (t, J=6.80, 3H). ¹³C NMR (CDCl₃): δ 168.5; 166.7; 145.4; 133.6; 130.8 (2C); 129.7; 129.2 (2C); 127.2 (2C); 116.1 (2C); 68.8; 61.4; 39.7; 32.0; 30.4; 27.2; 23.1; 14.7; 14.4. The reaction mixture was transferred to at 4 mL screw cap vial and was added lithium hydroxide monohydrate (7 mg, 0.2 and H₂O (0.3 mL). The mixture was capped and heated to 60° C. and agitated o.n. The reaction mixture was transferred to a separation funnel with DCM and was washed with water. The org. phases were combined and transferred to a PSA ion-exchange column, which was wash with MeOH several times. The product was released by treating the column with 1% TFA in MeOH. The filtrate was concentrated in vacuo yielding 6 mg (8%) of the title compound. LC/MS: Purity (UV/MS): 100/-.

### 4′[(4,4-Dicyclopropyl-3-butenyl)oxy][1,1-biphenyl]-4-carboxylic acid (26)

The title compound was prepared according to GP7 from (4-chloro-1-cyclopropyl-1-butenyl)cyclopropane (70 mL, 68 mg, 0.4 mmol) and 4′-Hydroxy-biphenyl-4-carboxylic acid ethyl ester (48 mg, 0.2 mmol). Yield: 31 mg (44%). LC/MS: Purity (UV/MS): 90/-.

### Example 16

### General Procedure 8 (GP8) (Based on General Scheme 3)

### 2-(2-Pyridinyl)ethyl 4-bromo-2-fluorobenzoate

In a dry, argon flushed round bottom flask 4-bromo-2-fluorobenzoic acid (4.5 mmol, 986 mg), 2-(2-hydroxyethyl)pyridine (6.75 mmol, 831 mg), EDCI (6.75 mmol, 1.29 g) and 1-hydroxybenzotriazole (6.75 mmol, 912 mg) was taken up in dry DMF (20 mL), the reaction mixture was cooled to 0° C. on an icebath under argon before adding DIPEA (6.75 mmol, 683 mg). The reaction mixture was allowed to warm to r.t. o.n., then taken up in EtOAc and washed with 5% citric acid, NaOH (1M), water and brine, then dried over Na₂SO₄ and concentrated in vacuo. Yield: 1.17 g (80%). ¹H NMR (400 MHz, CDCl₃) δ: 8.52 (d, J=3.2 Hz, 1H), 7.70 (t, J=8.0 Hz, 1H), 7.60-7.56 (m, 1H), 7.29-7.10 (m, 4H), 4.69 (t, J=6.7 Hz, 2H), 3.21 (t, J=6.7 Hz, 2H).

### 2-(2-Pyridinyl)ethyl 4-bromo-2-methylbenzoate

The title compound was prepared according to GP8 from 4-bromo-2-methylbenzoic acid (4.5 mmol, 968 mg) and 2-(2-hydroxyethyl)pyridine (6.75 mmol, 831 mg). Yield after extraction: 1.14 g (79%). ¹H NMR (400 MHz, CDCl₃) δ: 8.55 (d, J=2.8 Hz, 1H), 7.66-7.59 (m, 2H), 7.35-7.13 (m, 4H), 4.68 (t, J=6.6, 2H), 3.23 (t, J=6.6, 2H), 2.46 (s, 3H).

### 2-Cyanoethyl 4-bromo-3-methylbenzoate

The title compound was prepared according to GP8 from 4-bromo-3-methylbenzoic acid (4.5 mmol, 968 mg) and 3-hydroxypropionitrile (6.75 mmol, 480 mg). Yield after extraction: 1.10 g (91%). ¹H NMR (400 MHz, CDCl₃) δ: 7.89 (d, J=1.2 Hz, 1H), 7.70 (dd, J=1.2 and 8.4 Hz, 1H), 7.60 (d, J=8.4 Hz, 1H), 4.51 (t, J=6.3, 2H), 2.83 (t, J=6.3, 2H), 2.44 (s, 3H).

### Example 17

### General Procedure 9 (GP9) (Based on General Scheme 4)

### 2-(2-Pyridinyl)ethyl 4-heptyl-2-methyl[1,1′-biphenyl]-4-carboxylate

In a dry and argon flushed vial 1-bromo-4-n-octylbenzene (0.30 mmol, 77 mg) was taken up in dry THF (0.5 mL), the vial was cooled to −20° C. before slow addition of tBuLi (1.6 M, 0.60 mmol, 0.40 mL). After 1 h at −20° C. ZnBr₂ (1.5 M, 0.33 mmol, 0.22 mL) was added and the cooling stopped. In another dry and argon flushed vial Pd₂ dba₃ (0.015 mmol, 14 mg) and tfp (0.2 mmol, 14 mg) was taken up in dry NMP (0.5 mL), the activated catalyst was added to the zinc reagent via a syringe. Finally and 2-(2-pyridinyl)ethyl 4-bromo-3-methylbenzoate (0.2 mmol, 64 mg)was taken up in dry THF (0.5 mL) and added to the reaction mixture. The reaction was then heated to 50° C. for 16 h. After cooling to r.t., the reaction was quenched with NH₄Cl (aq.) and poured onto a hydromatrix, then extracted with EtOAc and concentrated in vacuo. 20 mg of crude product was purified by prep. LC/MS. Yield: 5.0 mg. LC/MS: purity (UV/MS): 100/97.

### 3-Fluoro-4′-octyl-biphenyl-4-carboxylic acid 2-pyridin-2-yl-ethyl ester (Compound of Formula 5)

The title compound was prepared according to GP9 from 1-bromo-4-n-octylbenzene (0.30 mmol, 81 mg) and 4-bromo-2-fluoro-benzoic acid 2-pyridin-2-yl-ethyl ester (0.2 mmol, 65 mg). Yield: 1.6 mg. LC/MS: purity (UV/MS): 80/80.

### 3-Fluoro-4′-heptyl-biphenyl-4-carboxylic acid 2-pyridin-2-yl-ethyl ester (Compound of Fomula 7)

The title compound was prepared according to GP9 from 1-bromo-4-n-heptylbenzene (0.30 mmol, 76 mg) and 4-bromo-2-fluoro-benzoic acid 2-pyridin-2-yl-ethyl ester (0.2 mmol, 65 mg). Yield: 1.6 mg. ¹H NMR (400 MHz, CDCl₃) δ: 8.60-8.54 (m, 1H), 7.95-7.86 (m, 1H), 7.67-7.58 (m, 1H), 7.54-7.47 (m, 2H), 7.43-7.36 (m, 1H), 7.36-7.20 (m, 4H), 7.19-7.13 (m, 1H), 4.74 (t, J=6.5, 2H), 3.27 (t, J=6.5, 2H), 2.65 (t, J=7.7, 2H), 1.71-1.59 (H, 2H), 1.42-1.18 (m, 8H), 0.89 (t, J=5.3, 3H).

LC/MS: purity (UV/MS): 70/70.

### Example 20

### General Procedure 12 (GP12) (Based on General Scheme 7)

### 4′-Octyl[1,1′-biphenyl]-4-carboxylic acid (Compound of Formula 34) (Compound of Formula 53)

Methyl 4′-octyl[1,1′-biphenyl]-4-carboxylate (0.2 mmol crude) was taken up in THF (1 mL) and water (0.5 mL), then LiOH (0.6 mmol, 20 mg) was added and the reaction heated to 80° C. o.n. on a shaker. After cooling the reaction mixture was poured onto a hydromatrix and extracted with EtOAc and concentrated in vacuo. 20 mg of crude product was purified by prep. LC/MS. Yield: 2.4 mg. LC/MS: purity (UV/MS): 100/100.

### 4′-Heptyl[1,1′-biphenyl]-4-carboxylic acid (Compound of Formula 10)

The title compound was prepared according to GP12 from methyl 4′-heptyl[1,1′-biphenyl]-4-carboxylate (0.2 mmol crude). Yield, 3.5 mg. LC/MS: purity (UV/MS): 92/96.

### 4′-(2-Butoxyethoxy)-2′,5′-dimethyl[1,1-biphenyl]-4-carboxylic acid (Compound of Formula 44)

The title compound was prepared according to GP12 from methyl 4′-(2-butoxyethoxy)-2′,5′-dimethyl[1,1′-biphenyl]-4-carboxylate (0.2 mmol crude). Yield: 4.0 mg. LC/MS: purity (UV/MS): 97/99.

### 4′-(Hexyloxy)-2′,5′-dimethyl[1,1′-biphenyl]-4-carboxylic acid (Compound of Formula 11)

The title compound was prepared according to GP12 from methyl 4′-(hexyloxy)-2′,5′-dimethyl[1,1′-biphenyl]-4-carboxylate (0.2 mmol crude). Yield: 3.5 mg. LC/MS: purity (UV/MS): 96/97.

### 2-Nitro-4′-octyl[1,1′-biphenyl]-4-carboxylic acid

The title compound was prepared according to GP12 from methyl 2-nitro-4′-octyl[1,1′-biphenyl]-4-carboxylicate (0.2 mmol crude). Yield: 2.4 mg. LC/MS: purity (UV/MS): 100/100.

### 4′-Heptyl-2-nitro[1,1′-biphenyl]-4-carboxylic acid

The title compound was prepared according to GP12 from methyl 4′-heptyl-2-nitro[1,1′-biphenyl]-4-carboxylicate (0.2 mmol crude). Yield: 5.2 mg. LC/MS: purity (UV/MS): 95/99.

### 3-Fluoro-4′-heptyl[1,1′-biphenyl]-4-carboxylic acid (Compound of Formula 36) (91aej50-5d)

The title compound was prepared according to GP12 from 2-pyridineethanol 3-fluoro-4′-heptyl[1,1′-biphenyl]-4-carboxylate (0.2 mmol crude). Yield: 3.2 mg. LC/MS: purity (UV/MS): 100/100.

### 3-Methyl-4′-octyl[1,1′-biphenyl]-4-carboxylic acid (Compound of Formula 55)

The title compound was prepared according to GP12 from 2-pyridineethanol 3-methyl-4′-octyl[1,1′-biphenyl]-4-carboxylate (0.2 mmol crude). Yield: 3.6 mg. LC/MS: purity (UV/MS): 100/100.

### 4-Heptyl-3-methyl[1,1′-biphenyl]-4-carboxylic acid (Compound of Formula 52)

The title compound was prepared according to GP12 from 2-pyridineethanol 4′-heptyl-3-methyl[1,1′-biphenyl]-4-carboxylate (0.2 mmol crude). Yield: 4.1 mg. LC/MS: purity (UV/MS): 95/100.

### 2-Methyl-4′-octyl[1,1′-hiphenyl]-4-carboxylic acid (Compound of Formula 1)

The title compound was prepared according to GP12 from 2-pyridineethanol 2-methyl-4′-octyl[1,1′-biphenyl]-4-carboxylate (0.2 mmol crude). Yield: 5.7 mg. LC/MS: purity (UV/MS): 99/100.

### 4-Heptyl-2-methyl[1,1′-biphenyl]-4-carboxylic acid

The title compound was prepared according to GP12 from 2-pyridineethanol 4′-heptyl-2-methyl[1,1′-biphenyl]-4-carboxylate (0.2 mmol crude). Yield: 3.4 mg. LC/MS: purity (UV/MS): 100/100.

### 2-Fluoro-4-octyl[1,1′-biphenyl]-4-carboxylic acid (Compound of Formula 38)

The title compound was prepared according to GP12 from 2-pyridineethanol 2-fluoro-4′-octyl[1,1′-biphenyl]-4-carboxylate (0.2 mmol crude). Yield: 4.1 mg. LC/MS: purity (UV/MS): 100/94.

### 3,5-Dimethyl-4′-octyl[1,1′-biphenyl]-4-carboxylic acid

The title compound was prepared according to GP12 from methyl 3,5-dimethyl-4′-octyl[1,1′-biphenyl]-4-carboxylate (0.2 mmol crude). Yield: 1.8 mg. LC/MS: purity (UV/MS): 100/93.

### 2-Fluoro-4′-heptyl[1,1′-biphenyl]-4-carboxylic acid (Compound of Formula 22)

The title compound was prepared according to GP12 from 2-pyridineethanol 2-fluoro-4′-heptyl[1,1′-biphenyl]-4-carboxylate (0.2 mmol crude). Yield: 2.8 mg. LC/MS: purity (UV/MS): 100/100.

### Example 21

### General Procedure 13 (GP13) (Based on General Scheme 8)

### 4′-(2-Butoxyethoxy)-[1,1-biphenyl]-4-carboxylic acid (Compound of Formula 18)

In a microwave vial ethyl 4′-hydroxy-[1,1′-biphenyl]-4-carboxylate (1 mmol, 242 mg), 2-butoxyethyl bromide (2 mmol, 362 mg), K₂CO₃ (2 mmol, 276 mg), KI (2 mmol, 332 mg) was taken up in dry MeCN (4 mL). The vial was capped and heated to 180° C. for 25 min. Filtered and concentrated onto celite, purified by flash chromatography (eluent 0-20% EtOAc in heptane) to yield ethyl 4′-(2-butoxyethoxy)-[1,1′-biphenyl]-4-carboxylate (280 mg, 82%). The ester (0.38 mmol, 130 mg) was cleaved according to GP12, purified by filtration after acidifying the reaction mixture with HCl (aq.) to yield the title compound as a white solid (105 mg, 87%). ¹H NMR (400 MHz, CDCl₃) δ: 7.84 (d, J=8.6 Hz, 2H), 7.41 (d, J=8.6 Hz, 2H), 7.35 (d, J=8.8 Hz, 2H), 6.79 (d, J=8.8 Hz, 2H), 3.94 (t, J=5.1, 2H), 3.59 (t, J=4.7, 2H), 3.34 (t, J=6.7, 2H), 1.40-1.36 (m, 2H), 1.20-1.14 (m, 2H), 0.71 (t, J=7.4, 3H).

¹³C NMR (100 MHz, CDCl₃) δ: 169.2, 159.4, 145.6, 132.9, 130.6, 128.9, 128.6, 126.7, 115.4, 71.7, 69.4, 67.8, 31.9, 19.4, 13.9. LC/MS: Purity (UV/MS): 98/-

### 4′-[2-(Hexylamino)-2-oxoethoxy]-[1,1′-biphenyl]-4-carboxylic acid

The title compound was prepared according to GP13 from ethyl 4′-hydroxy-[1,1′-biphenyl]-4-carboxylate (1 mmol, 242 mg) and 2-chloro-N-hexyl-acetamide (2 mmol, 354 mg). The intermediate ester was purified by flash chromatography (eluent 0-50% EtOAc in heptane) to yield ethyl 4′-[2-(hexylamino)-2-oxoethoxy]-[1,1′-biphenyl]-4-carboxylate (259 g, 68%). The ester (50 mg) was cleaved according to GP12, 20 mg of the crude product was purified by prep. LC/MS. Yield 6.3 mg. LC/MS: Purity (UV/MS): 100/43.

### Example 22

### General Procedure 14 (GP14) (Based on Scheme 8)

### 1-(Hexyloxy)-4-iodo-2,5-dimethylbenzene

2,5-Dimethyl-4-iodophenol (3.1 mmol, 765 mg) was taken up in MeCN (4 mL) and KOH (6.2 mmol, 360 mg) was added, the reaction mixture was left for two hours at 60° C., then KI (3.1 mmol, 520 mg) and 2-butoxyethyl bromide (6.2 mmol, 1.12 g) was added and the reaction was left for an additional 3 h. The reaction mixture was taken up in EtOAc, washed with water and dried over Na₂SO₄ and concentrated in vacuo. Purified by flash chromatography (eluent: 0-5% EtOAc in heptane) to yield the title compound (356 mg, 54%). ¹H NMR (400 MHz, CDCl₃) δ: 7.54 (s, 1H), 6.72 (s, 1H), 3.94 (t, J=6.4 Hz, 2H), 2.40 (s, 3H), 2.17 (s, 3H), 1.83-1.79 (m, 2H), 1.50-1.38 (m, 6H), 0.91 (bs, 3H).

¹³C NMR (100 MHz, CDCl₃) δ: 157.8, 140.2, 139.6, 126.9, 113.1, 89.1, 68.3, 31.8, 29.5, 28.2, 26.0, 22.9, 15.5, 14.3.

### 1-(2-Butoxyethoxy)-4-iodo-2,5-dimethylbenzene

The title compound was prepared according to GP14 from 2,5-dimethyl-4-iodophenol (3.1 mmol, 765 mg) and 1-iodohexane (6.2 mmol, 1.31 g). Purified by flash chromatography (eluent: 0-5% EtOAc in heptane) to yield the title compound (242 mg, 34%). ¹H NMR (400 MHz, CDCl₃) δ: 7.52 (s, 1H), 6.72 (s, 1H), 4.08 (t, J=5.0 Hz, 2H), 3.77 (t, J=5.0 Hz, 2H), 3.54 (t, J=6.6 Hz, 2H), 2.37 (s, 3H), 2.15 (s, 3H), 1.60-1.38 (m, 4H), 0.93 (t, J=7.5 Hz, 3H). ¹³C NMR (100 MHz, CDCl₃) δ: 157.6, 140.2, 139.6, 127.1, 113.5, 89.7, 71.6, 69.4, 68.2, 32.0, 28.2, 19.5, 15.5, 14.1.

### N-butyl-2-(4-iodo-2,5-dimethylphenoxy)acetamide

The title compound was prepared according to GP14 from 2,5-dimethyl-4-iodophenol (4.0 mmol, 992 mg) and 2-chloro-N-butulacetamide (8.0 mmol, 1.19 g). Purified by flash chromatography (eluent: 0-5% EtOAc in heptane) to yield the title compound (175 mg, 37%). ¹H NMR (400 MHz, CDCl₃) δ: 7.56 (s, 1H), 6.65 (s, 1H), 4.43 (s, 2H), 3.37-3.32 (m, 2H), 2.36 (s, 3H), 2.18 (s, 3H), 1.54-1.32 (m, 4H), 0.92 (t, J=7.2 Hz, 3H).

### Example 23

### General Procedure 15 (GP15) (Based on Scheme 8)

### 4-(4-Hexylcarbamoylmethoxy-5-methyl-thiazol-2-yl)-benzoic acid (Compound of Formula 13)

Methyl 4-(4-hydroxy-5-methyl-1,3-thiazol-2-yl)benzenecarboxylate (0.2 mmol, 50 mg), 2-chloro-N-hexylacetamide (0.4 mmol, 71 mg), potassium carbonate (0.4 mmol, 60 mg), potassium iodide (0.2 mmol, 33 mg) was transferred to a MW vial and 1 mL of CH₃CN was added. The vial was capped and the mixture was irradiated for 15 min at 180° C. The vial was decapped and lithium hydroxide monohydrate (0.5 mmol, 21 mg) and 0.3 mL H₂O were added. The mixture was heated to 70° C. for 3 days. The hydrolyzed reaction mixture was extracted with EtOAc and washed with water, aq. NaHCO₃, 4M HCl, H and brine. The organic layer was run through an anion exchange column (PSA). The ion exchange coumn was washed repeatedly with MeOH. After wash the ion exchange was treated with 10% TFA. The filtrate was collected and concentrated in vacuo, yielding the title compound (71 mg, 94%).

### 4-[4-(2-Butoxy-ethoxy)-5-methyl-thiazol-2-yl]-benzoic acid (Compound of Formula 27)

The title compound was prepared according to GP15 from Methyl 4-(4-hydroxy-5-methyl-1,3-thiazol-2-yl)benzenecarboxylate (0.2 mmol, 50 mg) and 2-butoxyethyl bromide (0.4 mmol, 72 mg) yielding the title compound (63 mg, 94%). LC/MS: Purity (UV/MS): 82/74.

### 4-[4-(2-Ethyl-hexyloxy)-5-methyl-thiazol-2-yl]-benzoic acid (Compound of Formula 46)

The title compound was prepared according to GP15 from Methyl 4-(4-hydroxy-5-methyl-1,3-thiazol-2-yl)benzenecarboxylate (0.2 mmol, 50 mg) and 2-ethylhexyl bromide (0.4 mmol, 77 mg) yielding the title compound (1 mg, 1%). LC/MS: Purity (UV/MS): 95/94.

### 4′-(5-Methyl-hexyloxy)-biphenyl-4-carboxylic acid (Compound of Formula 25)

The title compound was prepared according to GP15 from 4′-hydroxy-biphenyl-4-carboxylic acid ethyl ester (0.2 mmol, 48 mg) and 1-bromo-5-methylhexane (0.4 mmol, 72 mg, 0.05 mL) yielding the title compound (1 mg, 2%). LC/MS: Purity (UV/MS): 100/100.

### 4′-(4,4-Dicyclopropyl-but-3-enyloxy)-biphenyl-4-carboxylic acid (Compound of Formula 47)

The title compound was prepared according to GP15 from 4′-hydroxy-biphenyl-4-carboxylic acid ethyl ester (0.2 mmol, 48 mg) and 4-chloro-1,1-dicyclopropylbut-1-ene (0.4 mmol, 68 mg, 0.07 mL) yielding the title compound (31 mg, 44%). LC/MS: Purity (UV/MS): 90/32.

### Example 24

### General Procedure 16 (GP16) (Based on General Scheme 7 and 8)

### 4(5-Methyl-4-{[(4-methyl-cyclohexylmethyl)-carbamoyl]-methoxy}-thiazol-2-yl)-benzoic acid

An argon-flushed vial was charged with chloroacetyl chloride (1.1 mmol, 124 mg) and 1 mL of DCM. The solution was cooled to 0° C. and cyclohexylmethanamine (1.0 mmol, 113 mg) in 1 mL of DCM was added. The temperature was raised to r.t. and the reaction was agitated for 16 h. Then K₂CO₃ (2.0 mmol, 276 mg) was added and the mixture was agitated for another 2 h. The reaction mixture was quenched with water and extracted into DCM. The combined organic phases were washed with 2M HCl, H₂O, 2M NaOH and brine. The organic phase was concentrated in vacuo and transferred to a MW vial. Methyl 4-(4-hydroxy-5-methyl-1,3-thiazol-2-yl)benzenecarboxylate (0.2 mmol, 50 mg), potassium carbonate (0.4 mmol, 60 mg), potassium iodide (0.2 mmol, 33 mg) were added followed by addition of 1 mL of CH₃CN. The vial was capped and the mixture was irradiated for 15 min at 180° C. The vial was decapped and lithium hydroxide monohydrate (0.5 mmol, 21 mg) and 0.3 mL H₂O were added and the vial was irradiated for 7 min at 160° C. The reaction mixture was transferred to a hydromatrix that was pretreated with water and extracted with EtOAc. The filtrate was collected and concentrated in vacuo, and 20 mg of the concentrate was purified by prep LC/MS yielding the title compound (1 mg). LC/MS: Purity (UV/MS): 100/85.

### 4-(4-Cycloheptycarbamoylmethoxy-5-methyl-thiazol-2-yl)-benzoic acid (Compound of Formula 56)

The title compound was prepared according to GP16 from chloroacetyl chloride (1.1 mmol, 124 mg), cycloheptylamine (1.0 mmol, 113 mg) and methyl 4-(4-hydroxy-5-methyl-1,3-thiazol-2-yl)benzenecarboxylate (0.2 mmol, 50 mg). Purified by prep. LC/MS to yield the title compound (2 mg). LC/MS: Purity (UV/MS): 100/94.

### 4-(4-((isopentylcarbamoyl)methoxy)-5-methylthiazol-2-yl)benzoic acid

The title compound was prepared according to GP16 from chloroacetyl chloride (1.1 mmol, 124 mg), 3-methylbutan-1-amine (1.0 mmol, 87 mg) and methyl 4-(4-hydroxy-5-methyl-1,3-thiazol-2-yl)benzenecarboxylate (0.2 mmol, 50 mg). Purified by prep. LC/MS to yield the title compound (1 mg). LC/MS: Purity (UV/MS): 98/92.

### 4-(4-((cyclohexylcarbamoyl)methoxy)-5-methylthiazol-2-yl)benzoic acid

The title compound was prepared according to GP16 from chloroacetyl chloride (1.1 mmol, 124 mg), cyclohexylamine (1.0 mmol, 99 mg) and methyl 4-(4-hydroxy-5-methyl-1,3-thiazol-2-yl)benzenecarboxylate (0.2 mmol, 50 mg). Purified by prep. LC/MS to yield the title compound (1 mg). LC/MS: Purity (UV/MS): 99/79.

### 4-(4-Cyclopentylcarbamoylmethoxy-5-methyl-thiazol-2-yl)-benzoic acid (Compound of Formula 61)

The title compound was prepared according to GP16 from chloroacetyl chloride (1.1 mmol, 124 mg), cyclopentylamine (1.0 mmol, 85 mg) and methyl 4-(4-hydroxy-5-methyl-1,3-thiazol-2-yl)benzenecarboxylate (0.2 mmol, 50 mg). Purified by prep. LC/MS to yield the title compound (1 mg). LC/MS: Purity (LC/MS): 99/92.

### 4-[5-Methyl-4-[2-[(4-methylcyclohexyl)amino]-2-oxoethoxy]-2-thiazolyl]-benzoic acid, (Compound of Formula 58)

The title compound was prepared according to GP16 from chloroacetyl chloride (1.1 μmol, 124 mg), 4-methylcyclohexylamine (1.0 mmol, 113 mg) and methyl 4-(4-hydroxy-5-methyl-1,3-thiazol-2-yl)benzenecarboxylate (0.2 mmol, 50 mg). Purified by prep. LC/MS to yield the title compound (1 mg). LC/MS: Purity (UV/MS): 97/88.

### 4-[4-[2-[(1,4-Dimethylpentyl)amino]-2-oxoethoxy]-5-methyl-2-thiazolyl]-benzoic acid (Compound of Formula 57)

The title compound was prepared according to GP16 from chloroacetyl chloride (1.1 mmol, 124 mg), 5-methylhexan-2-amine (1.0 mmol, 115 mg) and methyl 4-(4-hydroxy-5-methyl-1,3-thiazol-2-yl)benzenecarboxylate (0.2 mmol, 50 mg). Purified by prep. LC/MS to yield the title compound (3 mg). LC/MS: Purity (UV/MS): 100/98.

### Example 25

### General Procedure 17 (GP17) (Based on General Scheme 7 and 8)

### 4-{4-[2-(2-Ethoxy-ethoxy)-ethoxy]-5-methyl-triazol-2-yl}-benzoic acid (Compound of Formula 64)

Methyl 4-(4-hydroxy-5-methyl-1,3-thiazol-2-yl)benzenecarboxylate (0.2 mmol, 50 mg), 1-(2-bromoethoxy)-2-ethoxyethane (0.4 mmol, 79 mg), potassium carbonate (0.4 mmol, 60 mg), potassium iodide (0.2 mmol, 33 mg) was transferred to a MW vial and 1 mL of CH₃CN was added. The vial was capped and the mixture was irradiated for 15 min at 180° C. The vial was decapped and lithium hydroxide monohydrate (0.5 mmol, 21 mg) and 0.3 mL H₂O were added and the vial was irradiated for 7 min at 160° C. The reaction mixture was transferred to a hydromatrix that was pretreated with water and extracted with EtOAc. The filtrate was collected and concentrated in Vacuo, and 20 mg of the concentrate was purified by prep LC/MS yielding the title compound (2 mg). LC/MS: Purity (UV/MS): 100/96.

### 4-[5-Methyl-4-(2-propoxy-ethoxy)-thiazol-2-yl]-benzoic acid (Compound of Formula 33)

The title compound was prepared according to GP17 from methyl 4-(4-hydroxy-5-methyl-1,3-thiazol-2-yl)benzenecarboxylate (0.2 mmol, 50 mg) and 2-chloroethyl N-propyl ether (0.4 mmol, 49 mg). Purified by prep. LC/MS to yield the title compound (2 mg). LC/MS: Purity (UV/MS): 100/96.

### 4-[4-[2-(2-Methoxyethoxy)ethoxy]-5-methyl-2-thiazolyl]-benzoic acid

The title compound was prepared according to GP17 from methyl 4-(4-hydroxy-5-methyl-1,3-thiazol-2-yl)benzenecarboxylate (0.2 mmol, 50 mg) and 1-bromo-2-(2-methoxyethoxy)ethane (0.4 mmol, 73 mg). Purified by prep. LC/MS to yield the title compound (8 mg). LCMS: Purity (UV/MS): 100/93.

### Example 26

### Based on General Scheme 3

### Imidazol-1-yl-(4-octyl-biphenyl-4-yl)-methanone (Compound of Formula 8)

4′-Octyl-4-biphenylcarboxylic acid (0.5 mmol, 155 mg) was added 3 mL THF and 1,1-Carbonyldiimidazole (2.5 mmol, 405 mg) and the mixture was heated to 66° C. for 4 days. The reaction mixture was cooled to rt and extracted with DCM and washed with water and brine. The combined organic phases were dried over Na₂SO₄, filtered and concentrated in vacuo, yielding the title compound (175 mg, 97%). %). ¹H NMR (400 MHz, CDCl₃) δ: 8.08 (s, 1H), 7.86-7.76 (m, 2H), 7.75-7.64 (m, 2H), 7.56-7.46 (m, 3H), 7.30-7.22 (m, 2H), 7-13 (s, 1H), 2.61 (t, J=7.3 Hz, 2H), 1.67-1.13 (m, 12H), 0.83 (t, J=6.6 Hz, 3H). ¹³C NMR (100 MHz, CDCl₃) δ: 166.1, 146.7, 144.0, 138.3, 136.7, 131.0, 130.6, 130.2, 129.3, 127.4, 127.3, 118.2, 35.8, 32.0, 31.5, 29.6, 29.5, 29.4, 22.8, 14.2.

### Example 27

### Based on General Scheme 3

### 4′-Heptyl-biphenyl-4-carboxylic acid hydroxyamide (Compound of Formula 37)

4′-Heptyl-4-biphenylcarboxylic acid (0.2 mmol, 50 mg) was added 1 mL thionyl chloride and was heated to 80° C. for 72 h. The resulting acid chloride was concentrated in vacuo and 1 mL of pyridine was added followed by addition of hydroxylamine hydrochloride (0.24 mmol, 18 mg). The reaction mixture was agitated 16 h at r.t. The mixture was transferred to a separation funnel with EtOAc and was washed with 2M NaOH and brine. The combined organic phases were dried over Na₂SO₄, filtered and concentrated in vacuo, yielding the title compound (5 mg, 10%). %). ¹H NMR (400 MHz, MeOD) δ: 7.88-7.74 (m, 2H), 7.74-7.63 (m, 2H), 7.61-7.50 (m, 2H), 7.33-7.21 (m, 2H), 2.64 (t, J=7.1 Hz, 2H), 1.74-1.56 (m, 10H), 0.89 (t, J=6.5 Hz, 3H).

### Example 28

### Based on General Scheme 3

### 4′-Heptyl-biphenyl-4-carboxylic acid hydrazide (Compound of Formula 45)

4′-Heptyl-4-biphenylcarboxylic acid (0.2 mmol, 50 mg) was added 1 mL thionyl chloride and was heated to 80° C. for 72 h. The resulting acid chloride was concentrated in vacuo and 1 mL of pyridine was added followed by addition of hydrazine monohydrate (12 μL, 0.24 mmol, 13 mg). The reaction mixture was agitated 16 h at r.t., then the temperature was raised to 80° C. and the mixture was agitated another 16 h. The mixture was transferred to a separation funnel with EtOAc and was washed with 2M NaOH and brine. The combined organic phases were dried over Na₂SO₄, filtered and concentrated in vacuo, yielding the title compound (9 mg, 17%). ¹H NMR (400 MHz, CDCl₃) δ 7.83-7.77 (m, 2H), 7.67-7.62 (m, 2H), 7.57-7.48 (m, 2H), 7.38 (s, 1H), 7.31-7.22 (m, 2H), 4.12 (s, 2H), 2.65 (t, J=7.3 Hz, 2H), 1.72-1.52 (m, 2H), 1.42-1.18 (m, 8H), 0.89 (t, J=4.2 Hz, 3H).

### Example 29

### Based on General Scheme 3

### N-(4′-Octyl-biphenyl-4-carbonyl)-methanesulfonamide (Compound of Formula 60)

4′-Octyl-4-biphenylcarboxylic acid (0.2 mmol, 50 mg) was added 1 mL thionyl chloride and was heated to 80° C. for 72 h. The resulting acid chloride was concentrated in vacuo and 1.0 mL of pyridine was added. This mixture was added to a solution of methane sulfonamide (0.15 mmol, 14 mg) in 0.5 mL of pyridine. The reaction mixture was agitated 16 h at r.t. The mixture was concentrated in vacuo, taken up in EtOAc and run through a PSA ion-exchange column. The filtrate was concentrated in vacuo, yielding the title compound (23 mg, 37%). ¹H NMR (400 MHz, CDCl₃) δ 7.98-7.87 (m, 2H), 7.76-7.64 (m, 2H), 7.58-7.45 (m, 2H), 7.34-7.23 (m, 2H), 3.45 (s, 3H), 2.66 (t, J=7.2 Hz, 2H), 1.73-1.57 (m, 2H), 1.46-1.17 (m, 10H), 0.89 (t, J=6.2, 3H). (100 MHz, CDCl₃) δ: 165.4, 146.8, 143.9, 136.7, 129.3, 129.3, 128.6, 127.5, 127.4, 42.0, 35.8, 32.0, 31.5, 29.6, 29.5, 29.4, 22.8, 14.2.

### Example 30

### N-Butyl-2-chloro-acetamide

An argon-flushed vial was charged with 10 mL of DCM and chloroacetyl chloride (5.5 mmol, 621 mg). The solution was cooled to 0° C. and n-butylamine (5.0 mmol, 366 mg) was slowly added. Then K₂CO₃ (5.5 mmol, 760 mg) was added and the mixture was agitated for 2 h. The reaction mixture was filtered and concentrated in vacuo. Yield: 584 mg (86%). ¹H NMR (400 MHz, CDCl₃) δ 6.60 (s, 1H), 4.00 (s, 2H), 3.32-3.22 (m, 2H), 1.56-1.44 (m, 2H), 1.40-1.16 (m, 4H), 0.91 (t, J=7.3, 3H), 0.89 (t, J=6.6, 3H). (100 MHz, CDCl₃) δ: 165.8, 42.8, 39.7, 31.5, 20.1, 13.7.

### Example 31

### Based on General Scheme 8

### 4-(4-Butylcarbamoylmethoxy-5-methyl-thiazol-2-yl)-benzoic acid (Compound of Formula 62)

A MW vial was charged with N-butyl-2-chloro-acetamide (83BG73-2) (0.6 mmol, 90 mg), methyl 4-(4-hydroxy-5-methyl-1,3-thiazol-2-yl)benzenecarboxylate (0.3 mmol, 75 mg), potassium carbonate (0.7 mmol, 90 mg) and potassium iodide (0.3 mmol, 55 mg) and 3 mL of DMF was added. The vial was capped and the mixture was irradiated for 15 min at 180° C. The vial was decapped and lithium hydroxide monohydrate (0.9 mmol, 38 mg) and 0.5 mL H₂O were added and the vial was capped and irradiated for 7 min at 160° C. The reaction mixture was transferred to a separation funnel and extracted into EtOAc. The organic phase was washed with 4% MgSO₄, 2M NaOH. The aqueous phase was added EtOAc and neutralized with 2M HCl, the organic phase was washed with brine, dried over Na₂SO₄, filtered and concentrated in vacuo, yielding the title compound (22 mg, 21%). LC/MS: Purity (UV/MS): 100/100.

### Example 32

### Based on General Scheme 2)

### 4-(4-Octyl-piperazin-1-yl)-benzoic acid (Compound of Formula 50)

1-(4-Cyanophenyl)-piperazine hydrochloride (1.0 mmol, 112 mg), 1-iodooctane (1.0 mmol, 240 mg), K₂CO₃ (1.0 mmol, 138 mg) and 1 mL of CH₃CN were transferred to a MW vial. The reaction mixture was irradiated for 10 min at 160° C. After cool down the reaction mixture was poured on to an unbuffered hydromatrix. The matrix was washed with EtOAc and the filtrate was concentrated in vacuo and purified by flash chromatography yielding 102 mg (72%). The alkylated product (0.1 mmol, 25 mg) was mixed with 0.2 mL of H₂O, 0.5 mL of 95-97% sulphuric acid and 0.5 mL of acetic acid and the resulting mixture was heated to 120° C. for 16 h. The reaction mixture was transferred to a separation funnel with EtOAc and water. The organic phase was washed with 2M NaOH and brine. The combined organic phases were dried over Na₂SO₄, filtered and concentrated in vacuo, yielding the title compound (17 mg, 64%). LC/MS: Purity UV/MS): 99/78.

### Example 33

### Based on General Scheme 6

### Cyanomethylene triphenylphosphine hydrochloride

Triphenylphosphine (105 mmol, 27.5 g) and chloroacetonitrile (100 mmol, 6.3 mL) were heated to reflux in toluene for 4 h then cooled to r.t. The precipitate was filtered and washed with 100 mL of heptane then dried under high vacuum o.n. to yielding the title compound as a white powder (18.5 g, 55%). %). ¹H NMR (400 MHz, CDCl₃) δ: 8.04-7.96 (m, 6H), 7.81-7.73 (m, 3H), 7.70-7.62 (m, 6H), 6.79-6.70 (m, 2H).

### Example 34

### Based on General Scheme 6)

### 3-(4-Octyl-biphenyl-4-yl)-3-oxo-2-(triphenyl-lambda*5*-phosphanylidene)-propionitrile

Cyanomethylene triphenylphosphine hydrochloride (3 mmol, 1.01 g) was dissolved in 20 mL of water and 20 mL of DMC, then NaOH (2M, 4.5 mL) was added. The reaction was swirled for 1 minute the layers separated and the aqueous phase was extracted with DCM (20 mL). This was dried over K₂CO₃ and filtered. 4′-Octyl-biphenyl-4-carboxylic acid (1.0 mmol, 310 mg) and EDCI.HCl (1.5 mmol, 288 mg) was added followed by DMAP (2 mg) and the reaction was stirred o.n. at r.t. 20 mL of water was added and the product was extracted into EtOAc. The product was washed with NaHCO₃, brine, dried over K₂CO₃, filtered and concentrated in vacuo to yield a thick oil which was purified by flash chromatography (eluent: 5-75% EtOAc in heptane) yielding the title compound (618 mg, 11%). ¹H NMR (400 MHz, CDCl₃) δ: 8.12-8.07 (m, 2H), 7.76-7.67 (m, 6H), 7.67-7.59 (m, 5H), 7.57-7.50 (m, 8H), 7.27-7.23 (m, 2H), 2.64 (t, J=7.2 Hz, 2H), 1.70-1.58 (m, 2H), 1.41-1.19 (m, 10H), 0.88 (t, J=6.8 Hz, 3H).

### Example 35

### Based on General Scheme 6

### (4′-Octyl-biphenyl-4-yl)-oxo-acetic acid (Compound of Formula 31)

3-(4′-Octyl-biphenyl-4-yl)-3-oxo-2-(triphenyl-lambda*5*-phosphanylidene)-propionitrile (0.03 mmol, 17 mg) was dissolved in 3 mL of DCM. To the mixture at r.t. and open to the air was added 0.5 mL DMDO in acetone. After a few drops the reaction went bright yellow, but this faded over a few minutes. 0.5 mL of H₂O was added and the reaction was stirred at r.t. for 5 min then concentrated in vacuo. The mixture was purified by flash chromatoghrapgy ((eluent: 0-40% EtOAc in heptane) yielding the title compound (5 mg, 54%). ¹H NMR (400 MHz, CDCl₃) δ: 8.11-8.06 (m, 2H), 7.64-7.59 (m, 2H), 7.51-7.46 (m, 2H), 7.24-7.17 (m, 2H), 2.61 (t, J=7.6 Hz, 2H), 1.63-1.53 (m, 2H), 1.34-1.12 (m, 10H), 0.82 (t, J=6.6 Hz, 3H).

### Example 36

### Based on General Scheme 8

### 1-(2-Butoxyethoxy)-4-iodobenzene

4-Iodo-phenol (3.0 mmol, 660 mg) and 1-(2-bromoethoxy)butane (4.5 mmol, 815 mg) were transferred to a 20 mL MW vial. 12 dry DMF was added followed by addition of Cs₂CO₃ (4.5 mmol, 1466 mg). The vial was capped and heated to 180° C. for 25 min by microwave irradiation. The reaction mixture was taken up in EtOAc, filtered through a plug of celite, was washed with 4% MgSO₄ and brine. The org. phases were collected, dried over MgSO₄, filtered and purified by flash chrom. (Hep:EtOAc 10:1). Yield: 915 mg (95%).

¹H NMR (300 MHz, CDCl₃) δ: 7.60-7.50 (m, 2H), 6.75-6.65 (m, 2H), 4.08 (t, J=0.6 Hz, 2H), 3.77 (t, J=5.0 Mz, 2H), 3.53 (t, J=6.6, 2H), 1.65-1.52 (m, 2H), 1.48-1.30 (m, 2H), 0.94 (t, J=7.3, 3H).

¹³C NMR (75 MHz, CDCl₃) δ: 158.7, 138.1, 117.1, 83.0, 71.5, 69.1, 67.7, 31.9, 19.5, 14.2.

### Example 37

### Based on General Scheme 3

### 2-(2-Pyridinyl)ethyl 4-bromo-2-fluorobenzoate

In a dry, nitrogen flushed round bottom flask 4-bromo-2-fluorobenzoic acid (4.0 mmol, 876 mg), 2-(2-hydroxyethyl)pyridine (6.0 mmol, 739 mg), EDCI.HCl (6.0 mmol, 1150 mg) and 1-hydroxybenzotriazole (6.0 mmol, 811 mg) were taken up in dry CH₃CN (20 mL) and DIPEA (6.75 mmol, 683 mg) was added. The reaction mixture was allowed to warm to r.t. o.n., then taken up in EtOAc and washed with 5% citric acid, NaOH (1M), water and brine, then dried over MgSO₄, filtered, concentrated in vacuo and purified by flash chrom. (p.ether/EtOAc 4:1-2:1). Yield: 1209 mg (93%).

¹H NMR (300 MHz, CDCl₃) δ: 8.58-8.52 (m, 1H), 7.78-7.68 (m, 1H), 7.68-7.57 (m, 1H), 7.33-7.14 (m, 4H), 4.72 (t, J=6.6 Hz, 2H), 3.25 (t, J=6.6 Hz, 2H).

¹³C NMR (75 MHz, CDCl₃) δ 163.5 (d, ¹J_{CF}=278.9 Hz), 163.3, 159.8 (da, ¹J^{CF}=278.9 Hz), 157.7, 149.4, 136.5, 133.1, 127.9 (d, ¹J_{CF}=9.5 Hz), 127.8 (d, ¹J_{CF}=9.5 Hz), 127.5 (d, ¹J_{CF}=3.7 Hz), 127.5 (d, ¹J_{CF}=3.7 Hz), 123.6, 121.8, 120.8 (d, ¹J_{CF}=25.5 Hz), 120.5 (d, ¹J_{CF}=25.5 Hz), 117.8 (d, ¹J_{CF}=9.8 Hz), 117.7 (d, ¹J_{CF}=9.8 Hz), 64.8, 37.4.

### Example 38

### Based on General Scheme 4

### 2-(2-Pyridinyl)ethyl 4′-(2-butoxyethoxy)-3-fluoro[1,1′-biphentyl]-4-carboxylate

In a dry and argon flushed vial 1-(2-butoxyethoxy)-4-iodobenzene (1.50 mmol, 480 mg) was taken up in dry THF (1.0 mL), the vial was cooled to 20° C. before slow addition of tBuLi (1.7 M, 3.0 mmol, 1.76 mL). After 1 h at −20° C., ZnBr₂ (1.5 M, 1.65 mmol, 1.10 mL) was added and the cooling stopped. In another dry and argon flushed vial Pd₂ dba₃ (0.05 mmol, 46 mg) and tfp (0.2 mmol, 46 mg) was taken up in dry NMP (1.5 mL), the activated catalyst was added to the zinc reagent via a syringe. Finally 2-(2-pyridinyl)ethyl 4-bromo-2-fluorobenzoate (1.0 mmol, 324 mg) was taken up in dry THF (1.5 mL) and added to the reaction mixture. The reaction was then stirred at r.t. for 16 h. The reaction was quenched with NH₄Cl (aq.), taken up in EtOAc and filtered through a plug of celite. The filtrate was washed with brine, dried over MgSO₄, filtered, concentrated in vacuo and purified by flash chrom. (p.ether/EtOAc 4:1-3:1). Yield: 370 mg (84%). ¹H NMR (300 MHz, CDCl₃) δ: 8.60-8.54 (m, 1H), 7.94-7.85 (m, 1H), 7.68-7.58 (m, 1H), 7.58-7.47 (m, 2H), 7.40-7.22 (m, 3H), 7.21-7.12 (m, 2H), 4.73 (t, J=6.7 Hz, 2H), 4.17 (t, J=4.2 Hz, 2H), 3.82 (t, J=4.7 Hz, 2H), 3.56 (t, J=6.6 Hz, 2H), 3.28 (t, J=6.4 Hz, 2H), 1.70-1.56 (m, 2H), 1.49-1.32 (m, 2H), 0.94 (t, J=7.3 Hz, 3H). LC/MS: purity (UV/MS): 100/100.

### Example 39

### Based on General Scheme 7

### 4′-(2-Butoxyethoxy)-3-fluoro[1,1′-biphenyl]-4-carboxylic acid

2-(2-pyridinyl)ethyl 4′-(2-butoxyethoxy)-3-fluoro[1,1′-biphenyl]-4-carboxylate (0.3 mmol, 131 mg) was taken up in THF (1 mL) and water (0.5 mL), then LiOH monohydrate (0.9 mmol, 38 mg) was added and the reaction heated by microwave irradiation at 160° C. for 5 min. After cooling the reaction mixture was taken up in EtOAc and was washed with water, 1M HCl and brine. The combined organic phases were dried over MgSO₄, filtered and concentrated in vacuo. Yield: 95 mg (95%). LC-MS: purity (UV/MS): 97/100. ¹H NMR (300 MHz, CDCl₃) δ: 10.59 (s, 1H), 8.10-7.99 (m, 1H), 7.60-7.49 (m, 2H), 7.46-7.38 (m, 1H), 7.38-7.28 (m, 1H), 7.07-6.96 (m, 2H), 4.19 (t, J=4.3 Hz, 2H), 3.84 (t, J=4.6 Hz, 2H), 3.58 (t, J=6.7 Hz, 2H), 1.71-1.56 (m, 2H), 1.50-1.33 (m, 2H), 0.95 (t, J=7.4 Hz, 3H). LC/MS: purity (UV/MS): 100/100.

### Example 40

### Receptor Selection and Amplification Technology Assay

The functional receptor assay, Receptor Selection and Amplification Technology (R-SAT), was used to investigate the pharmacological properties of known and novel RARβ agonists and antagonists. R-SAT is disclosed, for example, in U.S. Pat. Nos. 5,707,798, 5,912,132, and 5,955,281, Piu, F., Gauthier, N. K., and Wang, F., \Beta Arrestin 2 modulates the activity of Nuclear Receptor RAR beta 2 through activation of ERK2 kinase, Oncogen (2006) 25(2):218-29 and Burstein, E. S., Piu, F., Ma, J-N., Weissman, J. T., Currier, E. A., Nash, N. R., Weiner, D. M., Spalding, T. A., Schiffer, H. H., Del Tredici, A. L., Brann, M. R. Integrative Functional Assays, Chemical Genomics and High Throughput Screening: Harnessing signal transduction pathways to a common HTS readout Curr Pharm Des (2006) 12(14):1717-29 all of which are hereby incorporated herein by reference in their entireties, including any drawings.

These experiments have provided a molecular profile, or fingerprint, for each of these agents at the human RAR and RXR receptors. As can be seen in Table 1 and Table 2, these compounds of Formula I modulate the RARβ 2 receptor.

TABLE 1

RARβ2

| Compound no. | % Eff. | pEC50 |
|---|---|---|
| 1 | 39 | 8.64 |
| 2 | 126 | 8.10 |
| 3 | 107 | 8.02 |
| 4 | 44 | 7.82 |
| 6 | 104 | 7.73 |
| 8 | 79 | 7.66 |
| 9 | 76 | 7.59 |
| 10 | 64 | 7.58 |
| 11 | 78 | 7.56 |
| 12 | 72 | 7.54 |
| 13 | 85 | 7.38 |
| 15 | 76 | 7.37 |
| 16 | 37 | 7.34 |
| 18 | 108 | 7.32 |
| 20 | 98 | 7.26 |
| 22 | 36 | 7.24 |
| 24 | 58 | 7.21 |
| 25 | 65 | 7.20 |
| 26 | 36 | 7.15 |
| 27 | 95 | 7.12 |
| 29 | 78 | 7.08 |
| 31 | 80 | 7.02 |
| 32 | 70 | 7.02 |
| 33 | 98 | 6.96 |
| 35 | 83 | 6.94 |
| 36 | 42 | 6.91 |
| 37 | 49 | 6.87 |
| 38 | 70 | 6.81 |
| 44 | 41 | 6.61 |
| 45 | 78 | 6.59 |
| 46 | 54 | 6.58 |
| 47 | 58 | 6.58 |
| 49 | 59 | 6.55 |
| 50 | 85 | 6.53 |
| 51 | 59 | 6.51 |
| 52 | 45 | 6.41 |
| 53 | 99 | 6.29 |
| 54 | 39 | 6.18 |
| 55 | 84 | 6.17 |
| 56 | 105 | 6.17 |
| 57 | 77 | 6.17 |
| 58 | 66 | 6.15 |
| 59 | 35 | 6.11 |
| 60 | 51 | 6.08 |
| 61 | 39 | 6.08 |
| 62 | 37 | 6.08 |
| 63 | 36 | 6.05 |
| 64 | 77 | 6.00 |

Efficacy is relative to the maximal response of the reference ligand, Am-580.

TABLE 2

RARβ2

| Compound no. | % Eff. | pEC50 |
|---|---|---|
| 5 | 124 | 7.79 |
| 7 | 95 | 7.71 |
| 14 | 93 | 7.38 |
| 17 | 33 | 7.34 |
| 19 | 36 | 7.28 |
| 21 | 106 | 7.26 |
| 23 | 46 | 7.22 |
| 28 | 70 | 7.09 |
| 30 | 62 | 7.04 |
| 34 | 71 | 6.95 |
| 39 | 42 | 6.76 |
| 40 | 82 | 6.75 |
| 41 | 25 | 6.75 |
| 42 | 46 | 6.71 |
| 43 | 67 | 6.67 |
| 48 | 49 | 6.56 |

### Example 41

### Library Synthesis of Compounds

A library of compounds was synthesized by coupling a series of electrophiles and nucleophiles using the following scheme:

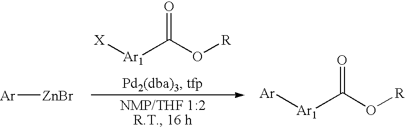

where Ar is a substituted aryl or heteroaryl in the nucleophiles and Ar₁ is an aryl or heteroaryl in the electrophile, R is an alkyl optionally substituted with an heteroaryl, and X is a halogen.

150 parallel reactions were conducted to obtain resulting diaryls. The nucleophiles used in the library synthesis included the following compounds:

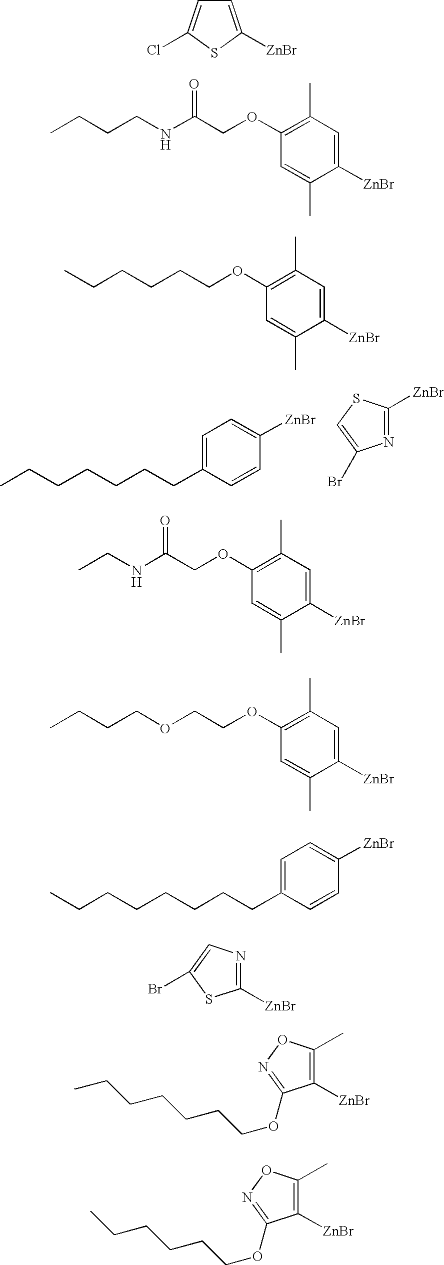

The electrophiles used in the synthesis included the following compounds:

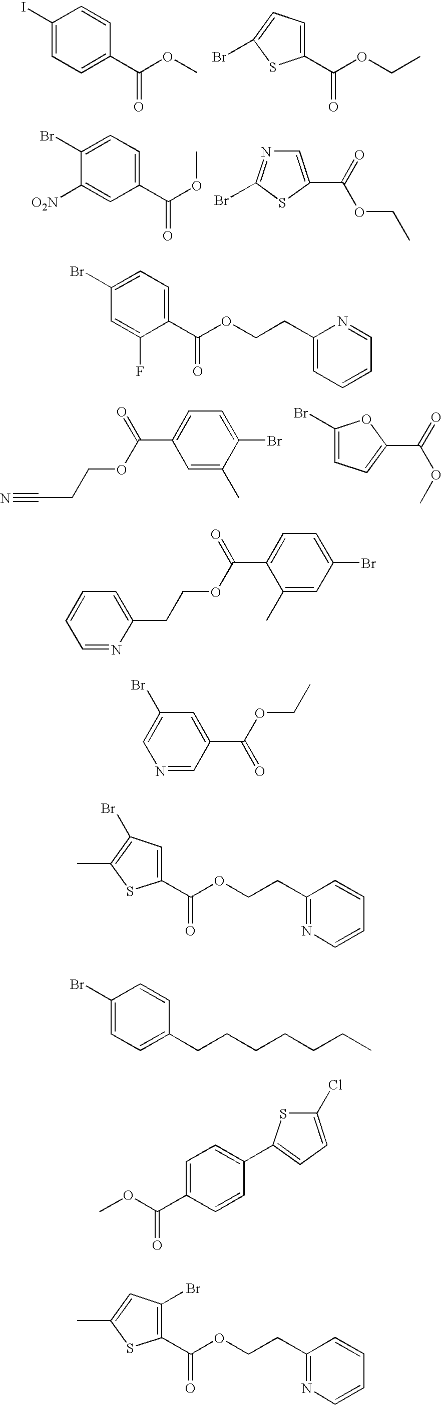

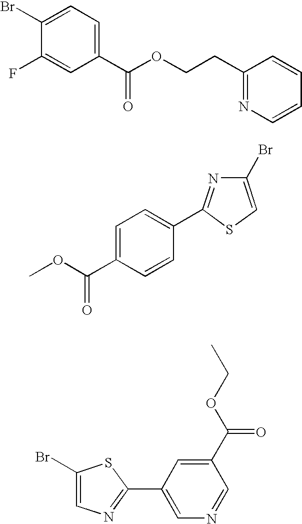

1. A compound of Formula I 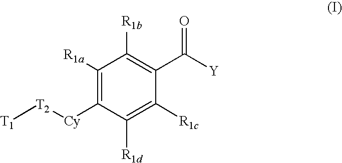
or a single isomer, mixture of isomers, racemic mixture of isomers, solvate, polymorph, metabolite, or pharmaceutically acceptable salt or prodrug thereof wherein:
R₁ₐ R_{1b}, R_{1c}, R_{1d} are independently selected from the group consisting of hydrogen, cyano, halogen, C₁₋₅ substituted or unsubstituted straight chained or branched alkyl, and substituted or unsubstituted cycloalkyl;
Cy is: 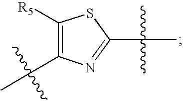
T₁ is selected from the group consisting of substituted or unsubstituted C₃-C₁₀ straight chained or branched alkyl, substituted or unsubstituted C₂-C₁₀ straight chained or substituted or unsubstituted branched alkenyl, C₂-C₁₀ straight chained or branched alkynyl, C₃-C₁₀ substituted or unsubstituted cycloalkyl, haloalkyl, —OR₂, —R₃OR₂, —OR₃OR₂, —N(R₂)(R₂ₐ), —C(═O)R₂, —C(═O)OR₂, —OC(═O)R₂, —C(═O)N(R₂)(R₂ₐ), —N(R₂)C(═O)(R₂ₐ), —N(R₂)C(═O)N(R₂ₐ)(R_{2b}), and —C═NN(R₂)(R₂ₐ);
T₂ is selected from the group consisting of C₂-C₁₀ unsubstituted straight chained or branched alkylene, C₃-C₁₀ substituted straight chained alkylene, C₄-C₁₀ substituted branched alkylene, C₂-C₁₀ substituted or unsubstituted straight chained or branched alkenylene, C₂-C₁₀ substituted or unsubstituted straight chained or branched acetylene, C₃-C₁₀ substituted or unsubstituted cycloalkylene, C₃-C₁₀ substituted or unsubstituted heterocycloalkylene, —OR₃—, —N(R₂)—, —C(═O)—, —C(═O)O—, —OC(═O)—, —C(═O)N(R₂)—, —N(R₂)C(═O)—, —N(R₂)C(═O)N(R₂)—, and —C═NN(R₂)—;
Y is selected from the group consisting of —OH, —NR₄R₄ₐ, —C(═O)OH, —OR₉, and —C(═O)OR₉;
R₄ and R₄ₐ are independently selected from the group consisting of hydrogen, —NH₂—OH, —SO₂CH₃, C₁-C₁₀ substituted or unsubstituted alkyl, substituted or unsubstituted heteroaryl, substituted or unsubstituted aryl, and substituted or unsubstituted heterocycle, or R₄ and R₄ₐ together form a C₃-C₈ heteroaryl optionally substituted with —NR₄C(═O)R₂;
R₅, is selected from the group consisting of hydrogen, optionally substituted C₁-C₅ straight chained alkyl or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, —CN, —C(═O)R₆, —C(═O)OR₆, —C(═O)N(R₆)R₆ₐ, —N(R₆)—C(═O)R₆ₐ, —N(R₆)—C(═O)N(R₆)R_{6b}, —N(R₆)—S(═O)₂R₆ₐ, —OC(═O)R₆, —S(═O)₂N(R₆)R₆ₐ, —S(═O)N(R₆)R₆ₐ, —SO₂R₆, and —SR₆; and
R₆, R₆ₐ and R_{6b} are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle;
R₂, R₂ₐ, and R_{2b} are independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, substituted or unsubstituted C₃-C₉ cycloalkyl, substituted or unsubstituted C₅-C₇ cycloalkenyl, substituted or unsubstituted aryl, and substituted or unsubstituted heteroaryl;
R₃ is selected from the group consisting of substituted or unsubstituted C₁-C₁₀ straight chained or branched alkylene, substituted or unsubstituted C₂-C₆ straight chained or branched alkenylene, C₂-C₆ substituted or unsubstituted straight chained or branched alkynylene, C₃-C₇ substituted or unsubstituted cycloalkylene, CH₂CH₂CH═C(CHCH₂CH₂)₂, and C₅-C₇ substituted or unsubstituted cycloalkenylene; and
R₉ is selected from C₁-C₂₀ substituted or unsubstituted, straight chained or branched alkyl, substituted or unsubstituted heteroaryl, substituted or unsubstituted heterocycle, substituted or unsubstituted cycloalkyl, and substituted or unsubstituted aryl.

2. A compound of Formula I 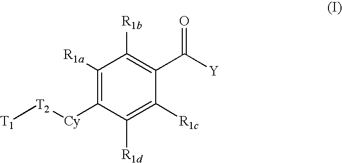
or a single isomer, mixture of isomers, racemic mixture of isomers, solvate, polymorph, metabolite, or pharmaceutically acceptable salt or prodrug thereof, wherein:
R₁ₐ R_{1b}, R_{1c}, R_{1d} are independently selected from the group consisting of hydrogen, cyano, halogen, C₁₋₅ substituted or unsubstituted straight chained or branched alkyl, and substituted or unsubstituted cycloalkyl;
Cy is: 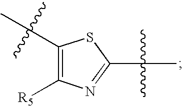
T₁ is selected from the group consisting of hydrogen, substituted or unsubstituted C₁-C₁₀ straight chained or branched alkyl, substituted or unsubstituted C₂-C₁₀ straight chained or substituted or unsubstituted branched alkenyl, C₂-C₁₀ straight chained or branched alkynyl, C₃-C₁₀ substituted or unsubstituted cycloalkyl, haloalkyl, —OR₂, —R₃OR₂, —OR₃OR₂, —N(R₂)(R₂ₐ), —C(═O)R₂, —C(═O)OR₂, —OC(═O)R₂, —C(═O)N(R₂)(R₂ₐ), —N(R₂)C(═O)(R₂ₐ), —N(R₂)C(═O)N(R₂ₐ)(R_{2b}), and —C═NN(R₂)(R₂ₐ);
T₂ is selected from the group consisting of C₃-C₁₀ substituted or unsubstituted straight chained or branched alkylene, C₂-C₁₀ substituted or unsubstituted straight chained or branched alkenylene, C₂-C₁₀ substituted or unsubstituted straight chained or branched acetylene, C₃-C₁₀ substituted or unsubstituted cycloalkylene, C₃-C₁₀ substituted or unsubstituted heterocycloalkylene, —O—, —OR₃—, —N(R₂)—, —OC(═O)—, —N(R₂)C(═O)—, —N(R₂)C(═O)N(R₂)—, and —C═NN(R₂)—;
Y is selected from the group consisting of —OH, —NR₄R₄ₐ, —C(═O)OH, —OR₉, and —C(═O)OR₉;
R₄ and R₄ₐ are independently selected from the group consisting of hydrogen, —NH₂, —OH, —SO₂CH₃, C₁-C₁₀ substituted or unsubstituted alkyl, substituted or unsubstituted heteroaryl, unsubstituted aryl, and substituted or unsubstituted heterocycle, or R₄ and R₄ₐ together form a C₃-C₈ heteroaryl optionally substituted with —NR₄C—O)R₂;
R₅, is selected from the group consisting of hydrogen, optionally substituted C₁-C₅ straight chained alkyl or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, —CN, —C(═O)R₆, —C(═O)OR₆, —C(═O)N(R₆)R₆ₐ, —N(R₆)—C(═O)R₆ₐ, —N(R₆)—C(═O)N(R₆ₐ)R_{6b}, —N(R₆)—S(═O)₂R₆ₐ, —OC(═O)R₆, —S(═O)₂N(R₆)R₆ₐ, —S(═O)N(R₆)R₆ₐ, —SO₂R₆, and —SR₆; and
R₆, R₆ₐ, and R_{6b} are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle;
R₂, R₂ₐ, and R_{2b} are independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, substituted or unsubstituted C₃-C₉ cycloalkyl, substituted or unsubstituted C₅-C₇ cycloalkenyl, substituted or unsubstituted aryl, and substituted or unsubstituted heteroaryl;
R₃ is selected from the group consisting of substituted or unsubstituted C₁-C₁₀ straight chained or branched alkylene, substituted or unsubstituted C₂-C₆ straight chained or branched alkenylene, C₂-C₆ substituted or unsubstituted straight chained or branched alkynylene, C₃-C₇ substituted or unsubstituted cycloalkylene, CH₂CH₂CH═C(CHCH₂CH₂)₂, and C₅-C₇ substituted or unsubstituted cycloalkenylene; and
R₉ is selected from C₁-C₂₀ substituted or unsubstituted, straight chained or branched alkyl, substituted or unsubstituted heteroaryl, substituted or unsubstituted heterocycle, substituted or unsubstituted cycloalkyl, substituted or unsubstituted phenyl, unsubstituted naphthalene, and substituted or unsubstituted azulene.

3. The compound according to claim 1, wherein said prodrug is selected from an ester derivative, amide derivative, carbohydroxamic acid derivative, imidazole derivative, carbohydrazide derivative, or peptide derivative of said compound.

4. The compound according to claim 1, wherein Y is —OR₉, C(═O)OH or —C(═O)OR₉.

5. The compound according to claim 1, selected from the group consisting of:

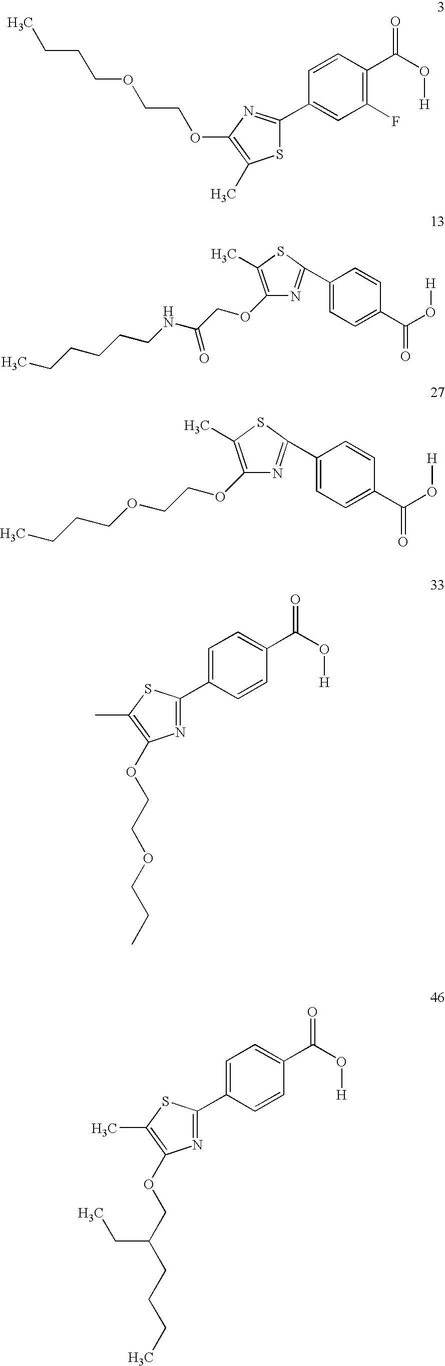

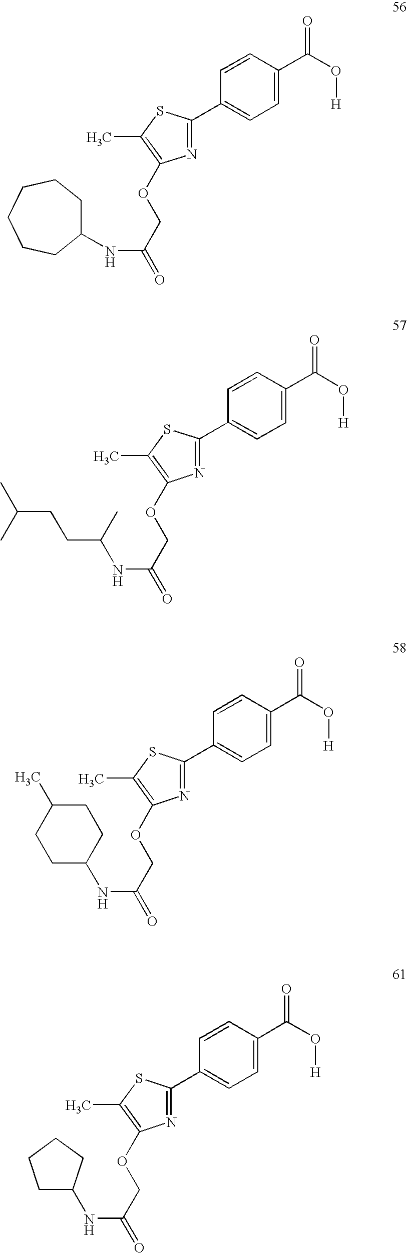

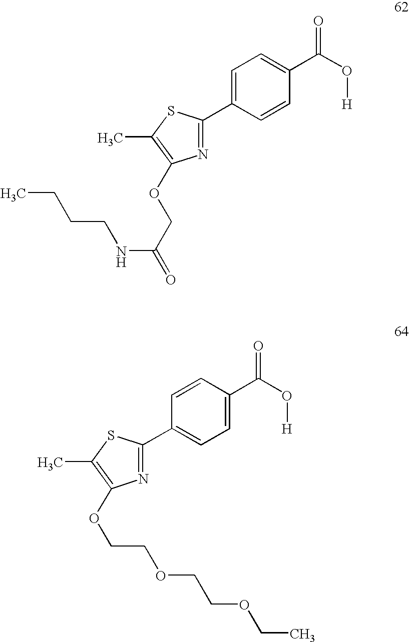

6. The compound according to claim 1, wherein said compound has activity at RARβ receptor subtypes.

7. The compound according to claim 1, wherein said compound has activity at the retinoic acid receptor subtype β isoform 2 (RARβ2).

8. A pharmaceutical composition comprising a compound for treating or alleviating symptoms of a disease or disorder associated with the RARβ receptor subtypes, wherein the compound is a compound of Formula I, 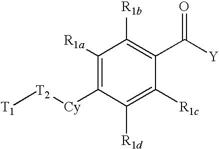
or a single isomer, mixture of isomers, racemic mixture of isomers, solvate, polymorph, metabolite, or pharmaceutically acceptable salt or prodrug thereof, wherein:
R₁ₐ, R_{1b}, R_{1c}, R_{1d} are independently selected from the group consisting of hydrogen, cyano, halogen, C₁₋₅ substituted or unsubstituted straight chained or branched alkyl, and substituted or unsubstituted cycloalkyl;
Cy is selected from the group consisting of: 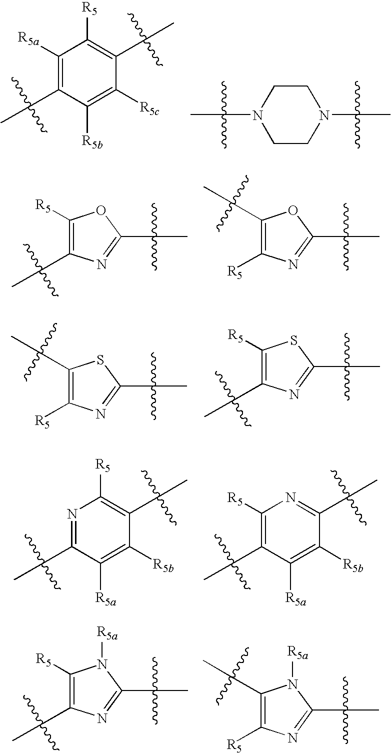
T₁ is selected from the group consisting of hydrogen, substituted or unsubstituted C₁-C₁₀ straight chained or branched alkyl, substituted or unsubstituted C₁-C₁₀ straight chained or substituted or unsubstituted branched alkenyl, C₁-C₁₀ straight chained or branched alkynyl, C₁-C₁₀ substituted or unsubstituted cycloalkyl, haloalkyl, —OR₂, —R₃OR₂, —OR₃OR₂, N(R₂)(R₂ₐ), —C(═O)R₂, —C(═O)OR₂, —OC(═O)R₂, —C(═O)N(R₂)(R₂ₐ), —N(R₂)C(═O)(R₂ₐ), —N(R₂)C(═O)N(R₂ₐ)(R_{2b}), and —C═NN(R₂)(R₂ₐ);
T₂ is selected from the group consisting of C₁-C₁₀ substituted or unsubstituted straight chained or branched alkylene, C₁-C₁₀ substituted or unsubstituted straight chained or branched alkenylene, C₁-C₁₀ substituted or unsubstituted straight chained or branched acetylene, C₁-C₁₀ substituted or unsubstituted cycloalkylene, C₁-C₁₀ substituted or unsubstituted heterocycloalkylene, —OR₃—, —O—, —N(R₂)—, —C(═O)—, —C(═O)O—, —OC(═O)—, —C(═O)N(R₂)—, —N(R₂)C(═O)—, —N(R₂)C(═O)N(R₂)—, and —C═NN(R₂)—;
Y is selected from the group consisting of —OH, —NR₄R₄ₐ, —C(═O)OH, —OR₉, and —C(═O)OR₉;
R₄ and R₄ₐ are independently selected from the group consisting of hydrogen, —NH₂, —OH, —SO₂CH₃, C₁-C₁₀ substituted or unsubstituted alkyl, substituted or unsubstituted heteroaryl, substituted or unsubstituted aryl, and substituted or unsubstituted heterocycle, or R₄ and R₄ₐ together form a C₃-C₈ heteroaryl optionally substituted with —NR₄C(═O)R₂;
R₅, R₅ₐ, R_{5b} and R_{5c} are independently selected from the group consisting of hydrogen, optionally substituted C₁-C₅ straight chained or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, —CN, —C(═O)R₆, —C(═O)OR₆, —C(═O)N(R₆)R₆ₐ, N(R₆)—C(═O)R₆ₐ, N(R₆)C(═O)N(R₆ₐ)R_{6b}, —N(R₆)—S(═O)₂ R₆ₐ —OC(═O)R₆, —S(═O)₂N(R₆)R₆ₐ, —S(═O)N(R₆)R₆ₐ, —SO₂R₆ and —SR₆; and
R₆, R₆ₐ and R_{6b} are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle
R₂, R₂ₐ, and R_{2b} are independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, substituted or unsubstituted C₃-C₉ cycloalkyl, substituted or unsubstituted C₅-C₇ cycloalkenyl, substituted or unsubstituted aryl, and substituted or unsubstituted heteroaryl;
R₃ is selected from the group consisting of substituted or unsubstituted C₁-C₁₀ straight chained or branched alkylene, substituted or unsubstituted C₂-C₆ straight chained or branched alkenylene, C₂-C₆ substituted or unsubstituted straight chained or branched alkynylene, C₃-C₇ substituted or unsubstituted cycloalkylene, CH₂CH₂CH═C(CHCH₂CH₂)₂, and C₅-C₇ substituted or unsubstituted cycloalkenylene;
R₉ is selected from C₁-C₂₀ substituted or unsubstituted, straight chained or branched alkyl, substituted or unsubstituted heteroaryl, substituted or unsubstituted heterocycle, substituted or unsubstituted cycloalkyl, and substituted or unsubstituted aryl;
and a pharmaceutically acceptable carrier.

9. The pharmaceutical composition according to claim 8, wherein Cy is selected from

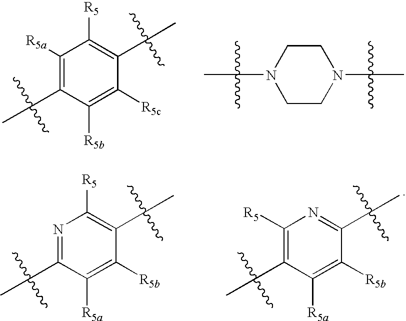

10. The pharmaceutical composition according to claim 9, wherein Cy is selected from:

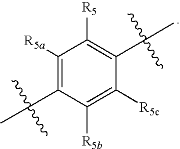

11. The pharmaceutical composition according to claim 9, wherein the compound is selected from

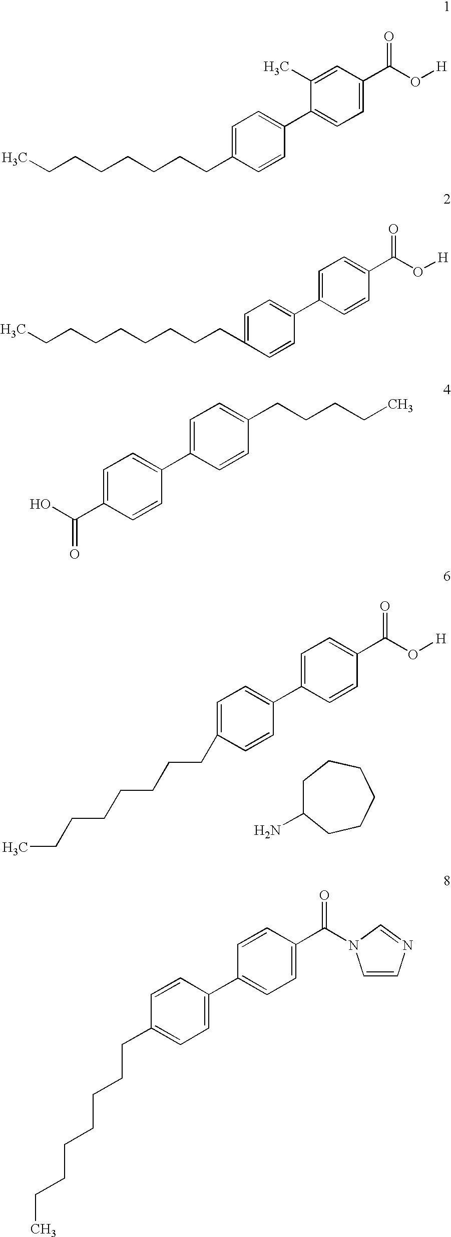

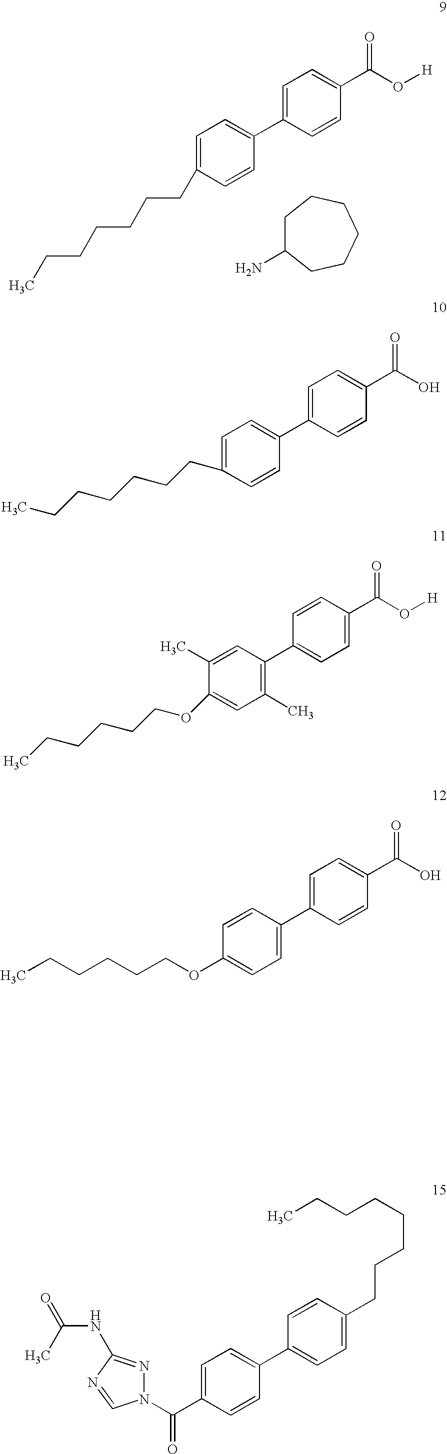

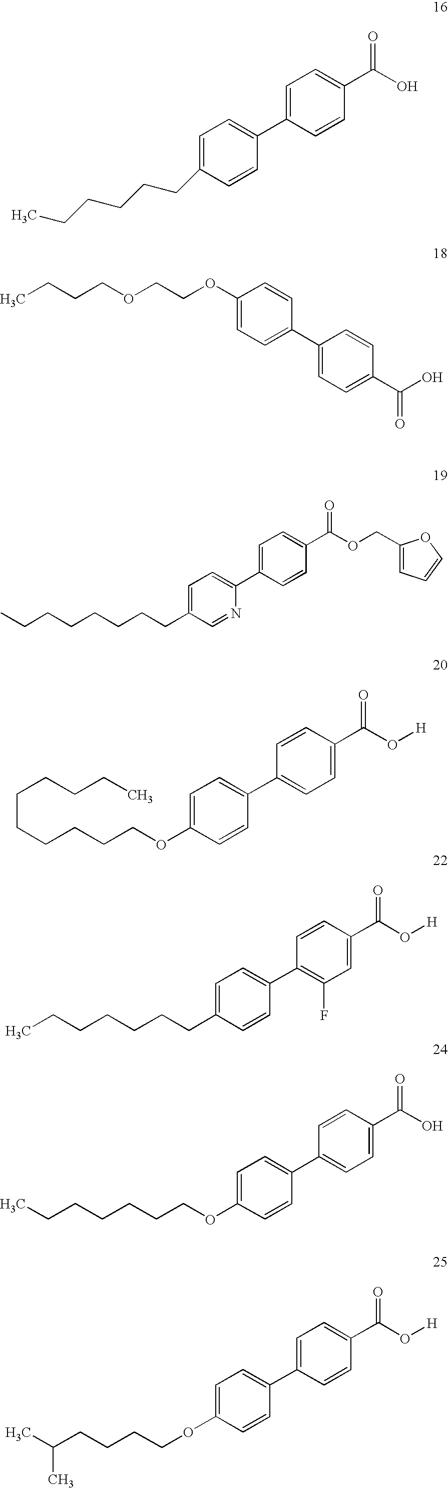

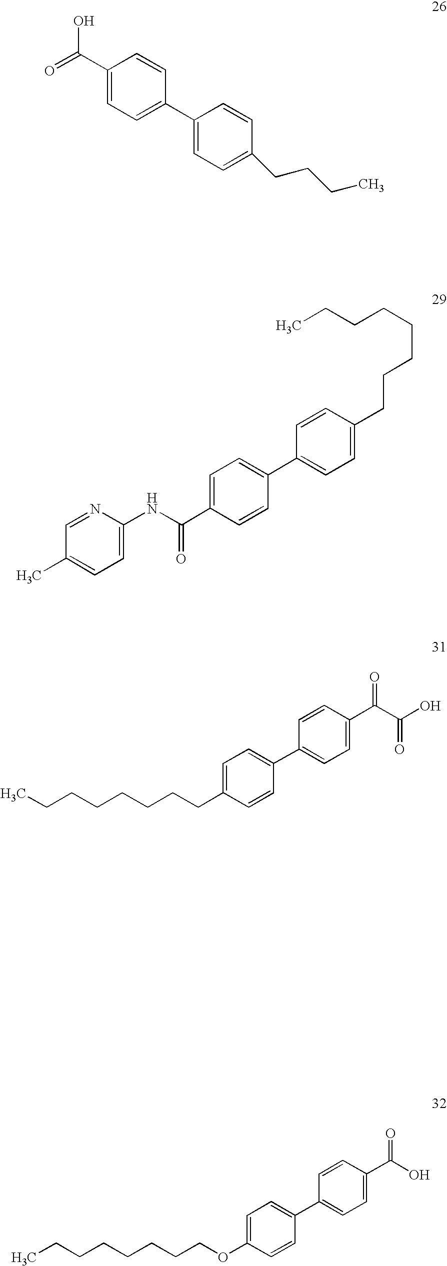

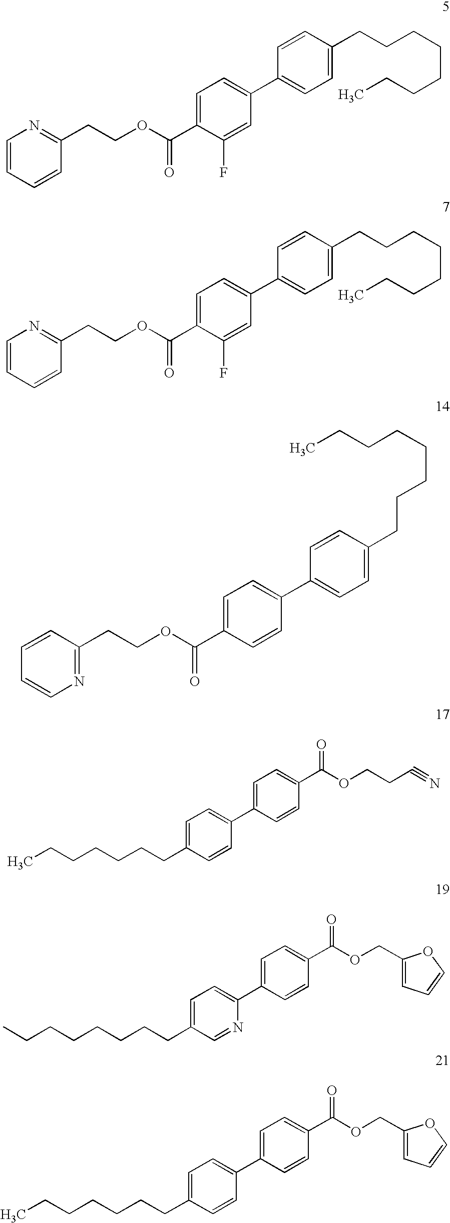

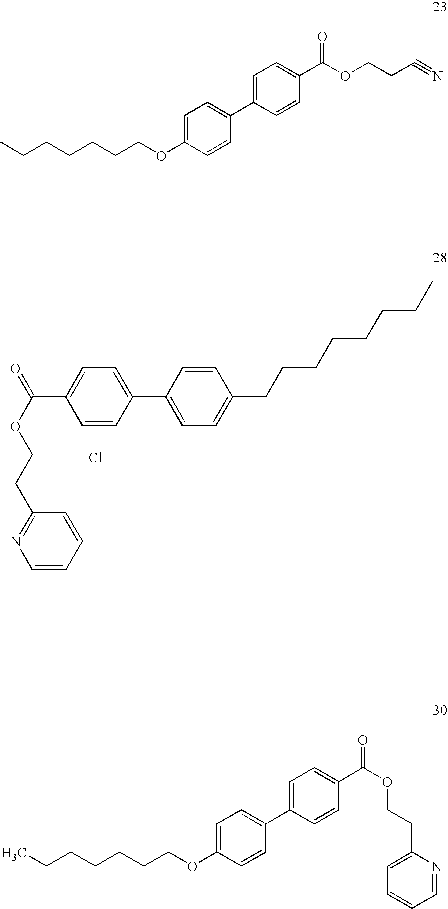

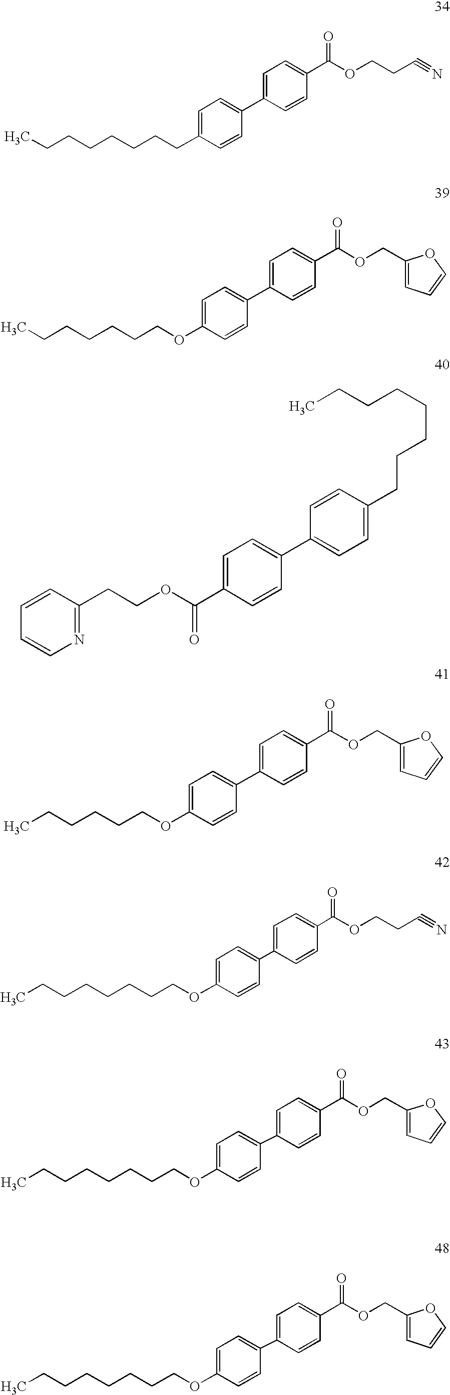

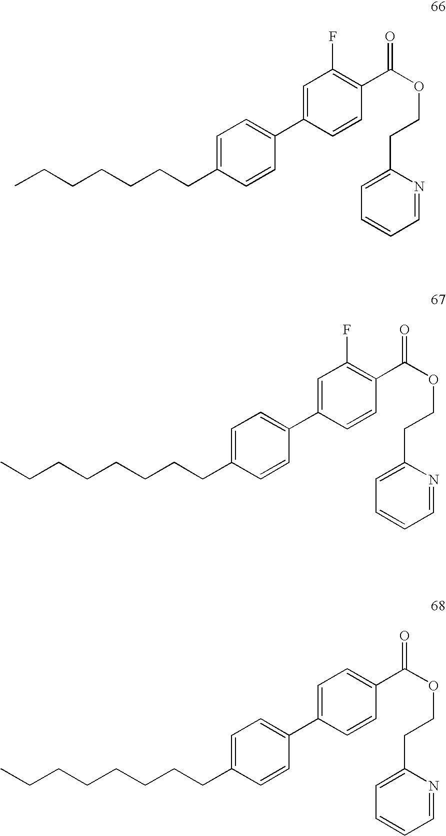

12. The pharmaceutical composition according to claim 8, wherein Cy is selected from

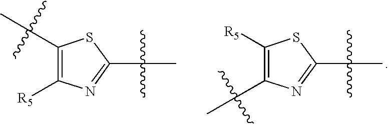

13. The pharmaceutical composition according to claim 12, wherein the compound is selected from

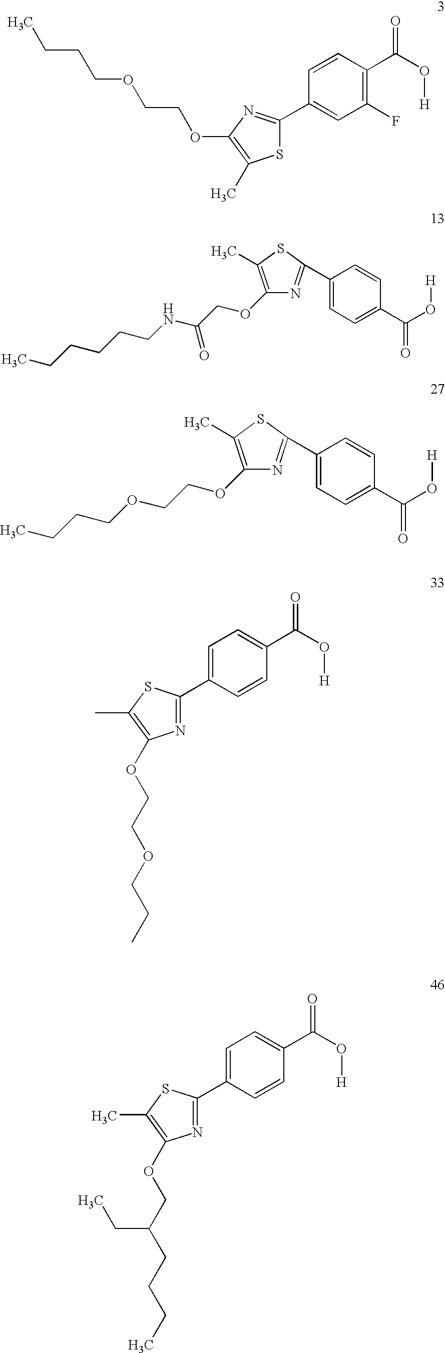

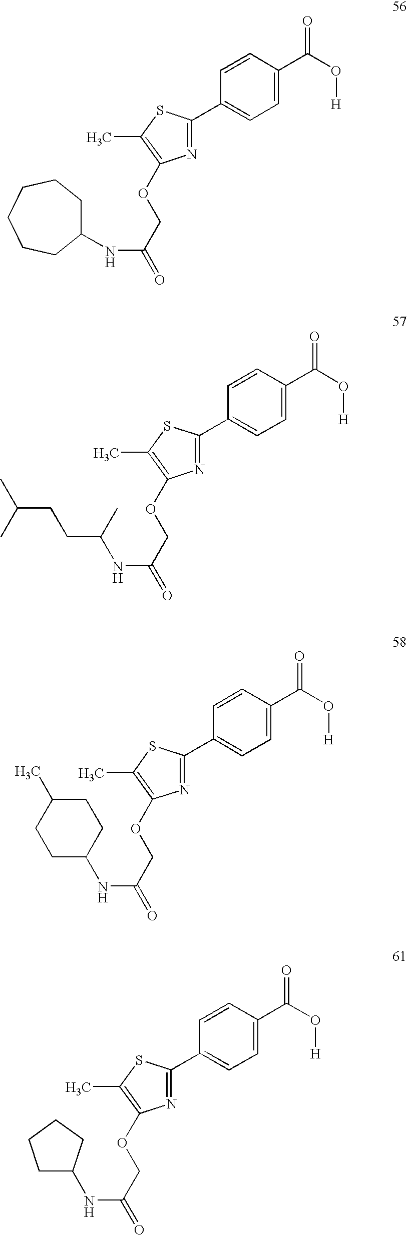

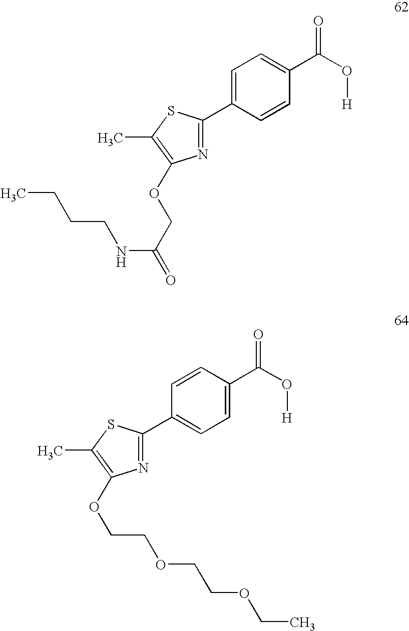

14. The pharmaceutical composition according to claim 9, wherein said RARβ receptor subtype is selected from isoform 2.

15. The pharmaceutical composition according to claim 9, wherein said symptoms, diseases or disorders are selected from cancer, a neurological disorder, a neurodegenerative disorder, an inflammatory disorder.

16. The pharmaceutical composition according to claim 15, wherein said cancer comprises a malignant tumor.

17. The pharmaceutical composition according to claim 15, wherein said cancer is selected from the group consisting of breast carcinoma and tumors in head, neck, lung, esophagus, mammary gland, pancreas, or cervix.

18. The pharmaceutical composition according to claim 15, wherein said neurological disorder is selected from the group consisting of performance deficits in spatial learning, memory tasks and age-related memory deficit, a disorder wherein cognition is altered, and schizophrenia.

19. The pharmaceutical composition according to claim 15, wherein said neurodegenerative disorder is Parkinson's disease, Alzheimer's disease, or a motor neuron disease.

20. The pharmaceutical composition according to claim 15, wherein said neurodegenerative disorder is caused by a stroke, nerve cell damage, nerve cell damage due to spinal cord injury, nerve cell damage due to damage of cardiac muscles, islet cell damage in diabetes, or multiple sclerosis.

21. A method for modulating a RARβ receptor using a compound, wherein the compound is a compound of Formula I, 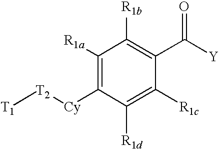
or a single isomer, mixture of isomers, racemic mixture of isomers, solvate, polymorph, metabolite, or pharmaceutically acceptable salt or prodrug thereof, wherein:
R₁ₐ R_{1b}, R_{1c}, R_{1d} are independently selected from the group consisting of hydrogen, cyano, halogen, C₁₋₅ substituted or unsubstituted straight chained or branched alkyl, and substituted or unsubstituted cycloalkyl;
Cy is selected from the group consisting of: 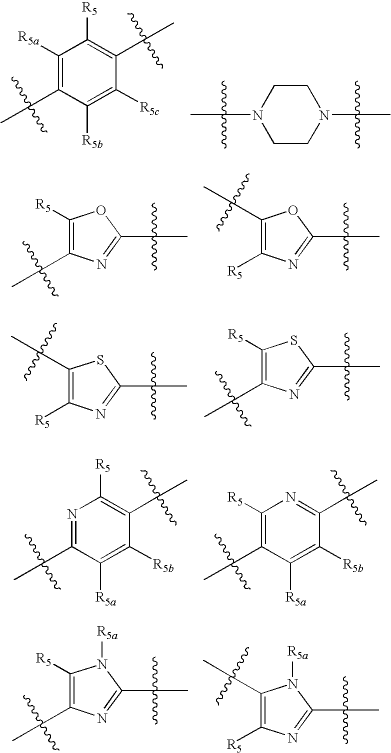
T₁ is selected from the group consisting of hydrogen, substituted or unsubstituted C₁-C₁₀ straight chained or branched alkyl, substituted or unsubstituted C₁-C₁₀ straight chained or substituted or unsubstituted branched alkenyl, C₁-C₁₀ straight chained or branched alkynyl, C₁-C₁₀ substituted or unsubstituted cycloalkyl, haloalkyl, —OR₂, —R₃OR₂, —OR₃OR₂, —N(R₂)(R₂ₐ), —C(═O)R₂, —C(═O)OR₂, —OC(═O)R₂, —C(═O)N(R₂)(R₂ₐ), —N(R₂)C(═O)(R₂ₐ), —N(R₂)C(═O)N(R₂ₐ)(R_{2b}), and —C═NN(R₂)(R₂ₐ);
T₂ is selected from the group consisting of C₁-C₁₀ substituted or unsubstituted straight chained or branched alkylene, C₁-C₁₀ substituted or unsubstituted straight chained or branched alkenylene, C₁-C₁₀ substituted or unsubstituted straight chained or branched acetylene, C₁-C₁₀ substituted or unsubstituted cycloalkylene, C₁-C₁₀ substituted or unsubstituted heterocycloalkylene, —OR₃—, —O—, —N(R₂)—, —C(═O)—, —C(═O)O—, —OC(═O)—, —C(═O)N(R₂)—, —N(R₂)C(═O)—, —N(R₂)C(═O)N(R₂)—, and —C═NN(R₂)—;
Y is selected from the group consisting of —OH, —NR₄R₄ₐ, —C(═O)OH, —OR₉, and —C(═O)OR₉;
R₄ and R₄ₐ are independently selected from the group consisting of hydrogen, —NH₂, —OH, —SO₂CH₃, C₁-C₁₀ substituted or unsubstituted alkyl, substituted or unsubstituted heteroaryl, substituted or unsubstituted aryl, and substituted or unsubstituted heterocycle, or R₄ and R₄ₐ together form a C₃-C₈ heteroaryl optionally substituted with —NR₄C(═O)R₂;
R₅, R₅ₐ, R_{5b} and R_{5c} are independently selected from the group consisting of hydrogen, optionally substituted C₁-C₅ straight chained or branched alkyl, optionally substituted C₂-C₅ straight chained or branched alkenyl, optionally substituted C₂-C₅ straight chained or branched alkynyl, optionally substituted C₃-C₆ cycloalkyl, hydroxy, nitro, amino, halogen, sulfonate, haloalkyl, —OR₆, —N(R₆)R₆ₐ, —CN, —C(═O)R₆, —C(═O)OR₆, —C(═O)N(R₆)R₆ₐ, —N(R₆)—C(═O)R₆ₐ, —N(R₆)—C(═O)N(R₆ₐ)R_{6b}, —N(R₆)—S(═O)₂R₆ₐ, —OC(═O)R₆, S(═O)₂N(R₆)R₆ₐ, —S(═O)N(R₆)R₆ₐ, —SO₂R₆, and —SR₆; and
R₆, R₆ₐ and R_{6b} are independently selected from the group consisting of hydrogen, C₁-C₅ straight chained or branched alkyl optionally substituted with an aryl or heteroaryl, C₂-C₅ straight chained or branched alkenyl optionally substituted with an aryl or heteroaryl, C₂-C₆ straight chained or branched alkynyl optionally substituted with an aryl or heteroaryl, C₃-C₆ cycloalkyl, and C₅-C₆ cycloalkenyl, or two of R₆, R₆ₐ and R_{6b} and the atom to which they are attached may together form a heterocycle
R₂, R₂ₐ, and R_{2b} are independently selected from the group consisting of hydrogen, C₁-C₁₀ straight chained or branched alkyl optionally substituted with an aryl or heteroaryl, C₂-C₁₀ straight chained or branched alkenyl optionally substituted with an aryl or heteroaryl, C₂-C₁₀ straight chained or branched alkynyl optionally substituted with an aryl or heteroaryl, substituted or unsubstituted C₃-C₉ cycloalkyl, substituted or unsubstituted C₅-C₇ cycloalkenyl, substituted or unsubstituted aryl, and substituted or unsubstituted heteroaryl;
R₃ is selected from the group consisting of substituted or unsubstituted C₁-C₁₀ straight chained or branched alkylene, substituted or unsubstituted C₂-C₆ straight chained or branched alkenylene, C₂-C₆ substituted or unsubstituted straight chained or branched alkynylene, C₃-C₇ substituted or unsubstituted cycloalkylene, CH₂CH₂CH═C(CHCH₂CH₂)₂, and C₅-C₇ substituted or unsubstituted cycloalkenylene; and
R₉ is selected from C₁-C₂₀ substituted or unsubstituted, straight chained or branched alkyl, substituted or unsubstituted heteroaryl, substituted or unsubstituted heterocycle, substituted or unsubstituted cycloalkyl, and substituted or unsubstituted aryl.

22. The method for modulating a RARβ receptor according to claim 21, wherein Cy is selected from

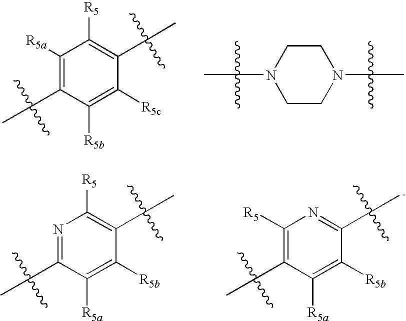

23. The method for modulating a RARβ receptor according to claim 22, wherein Cy is selected from

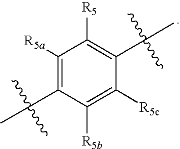

24. The method for modulating a RARβ receptor according to claim 22, wherein the compound is selected from

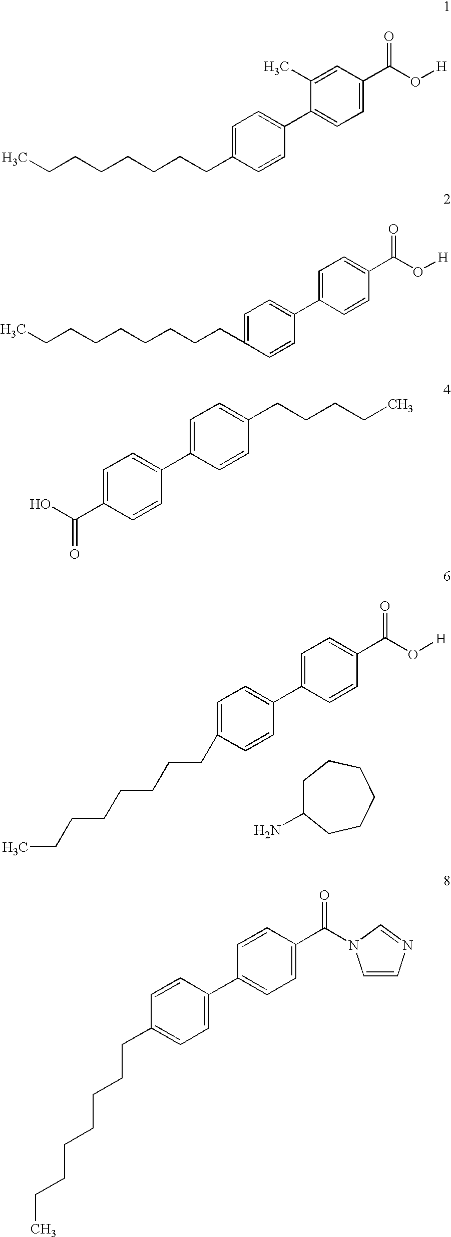

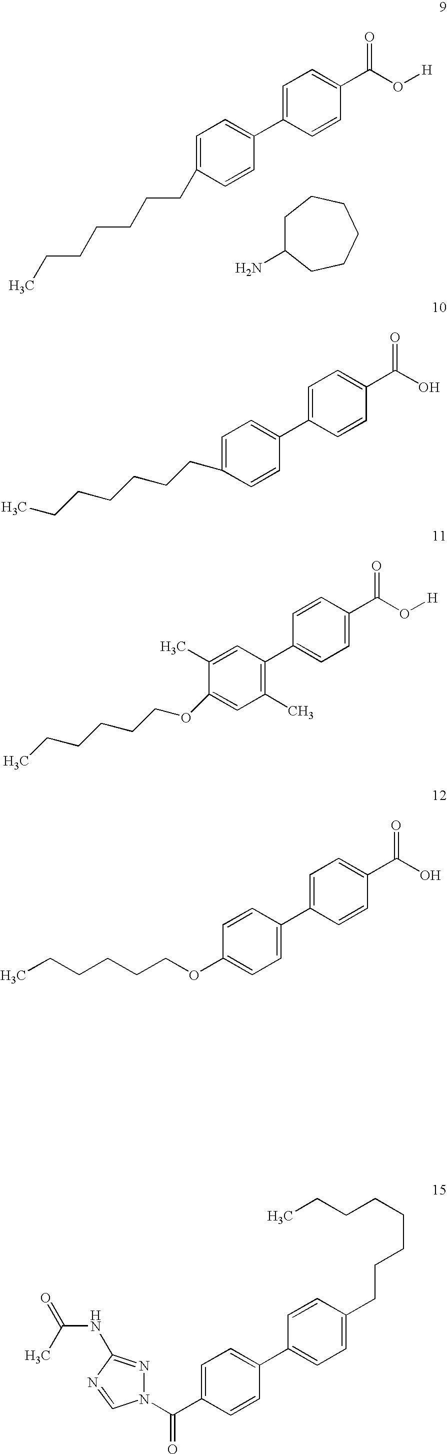

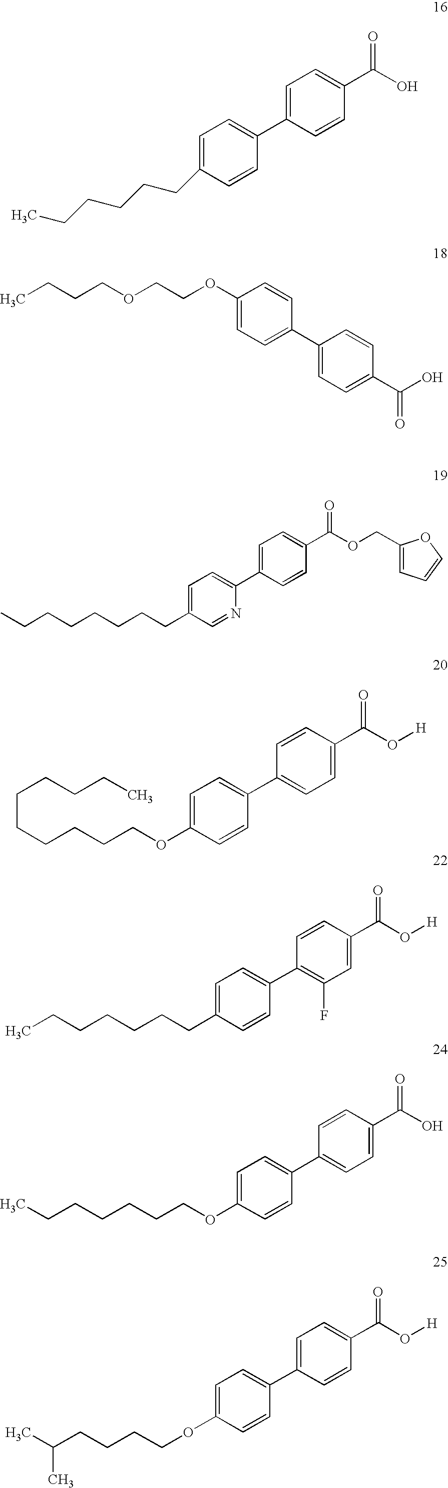

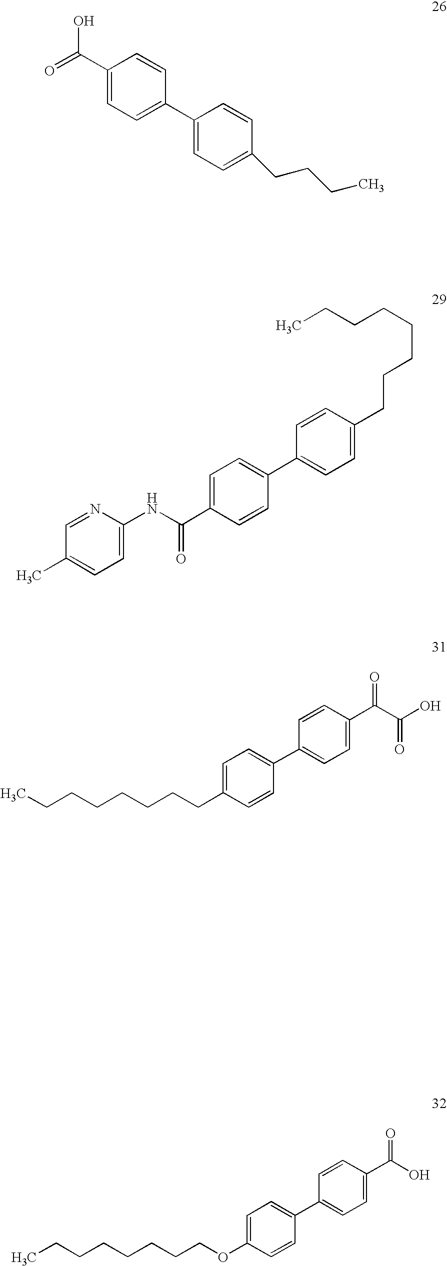

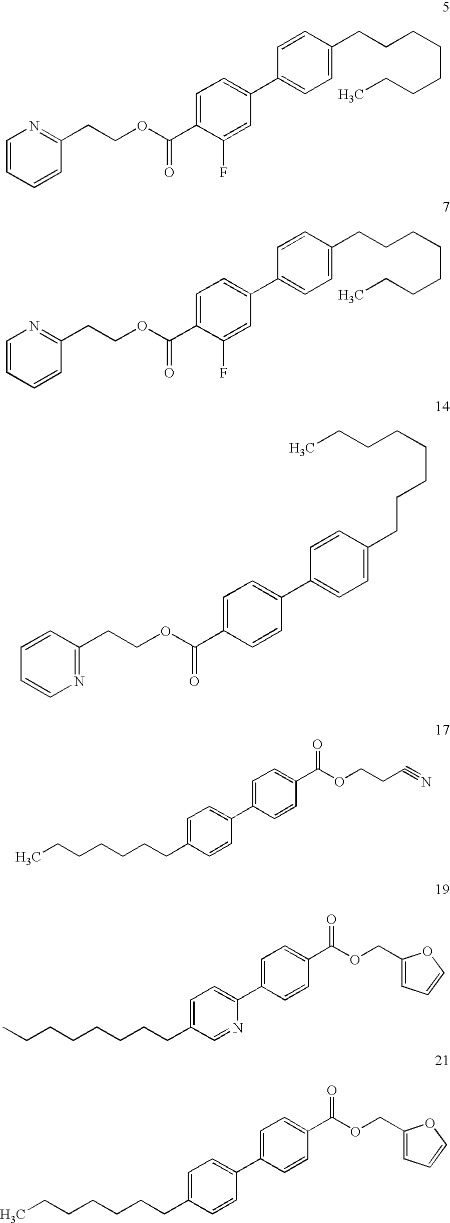

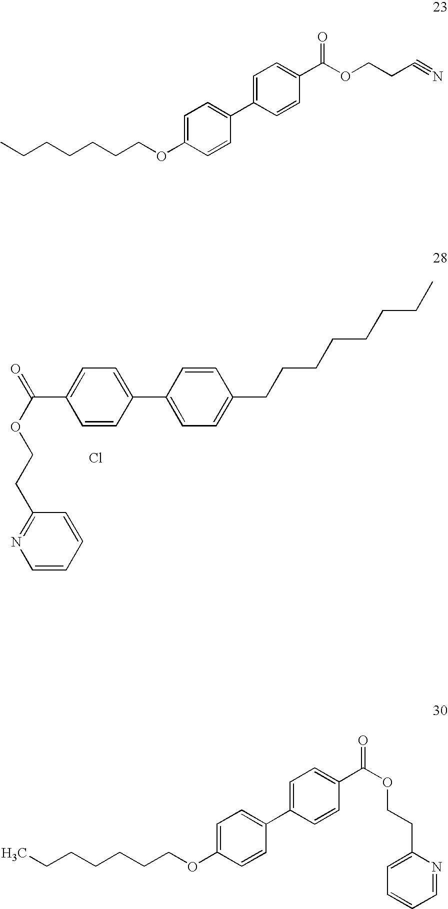

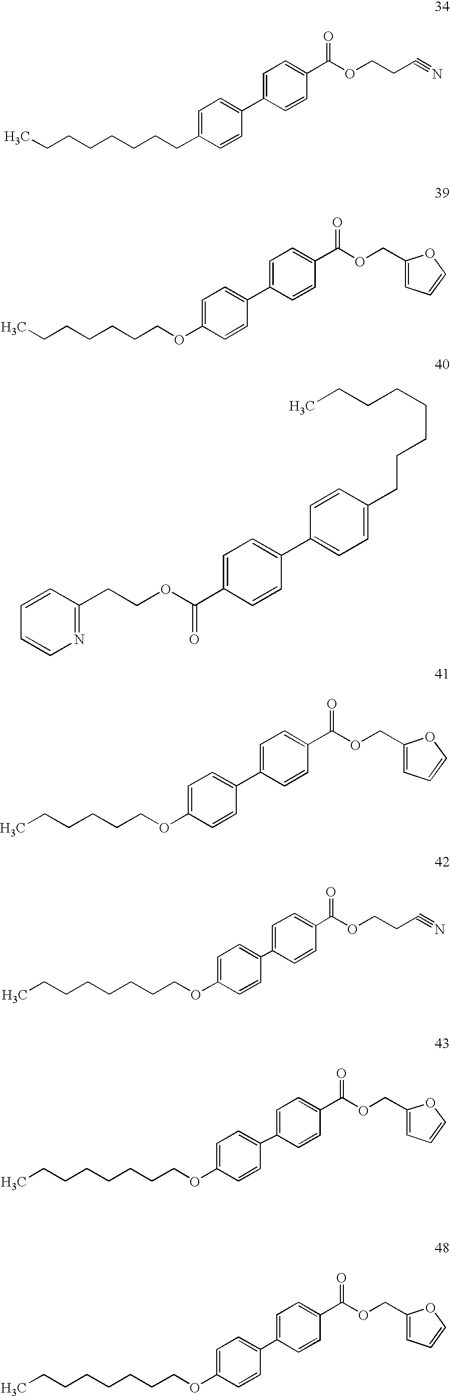

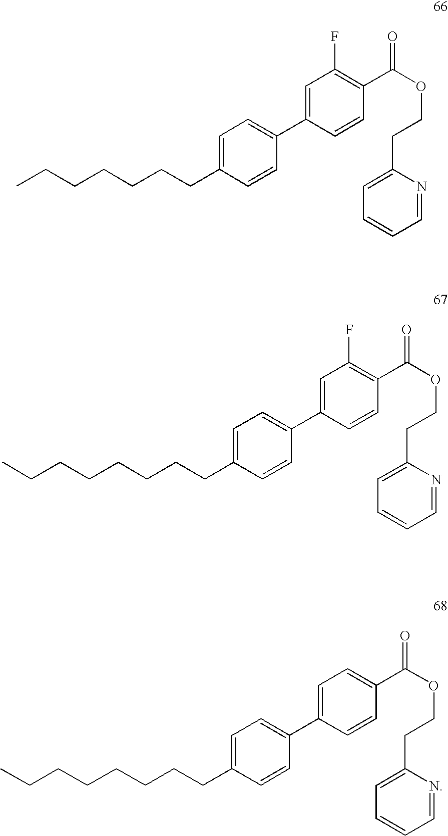

25. The method for modulating a RARβ receptor according to claim 21, wherein Cy is selected from

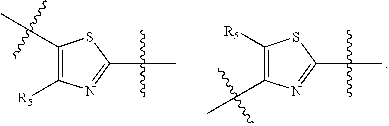

26. The method for modulating a RARβ receptor according to claim 25, wherein the compound is selected from

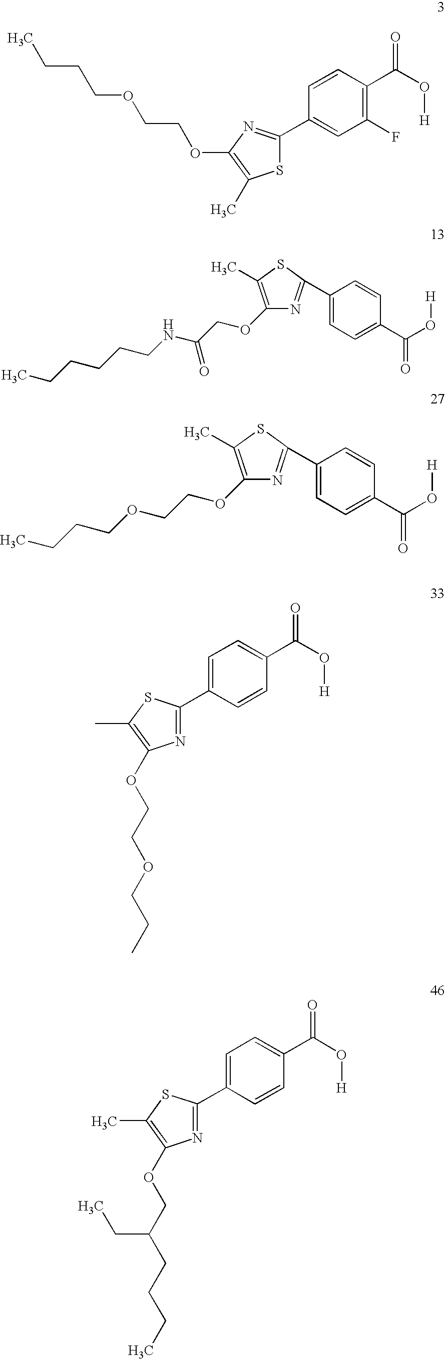

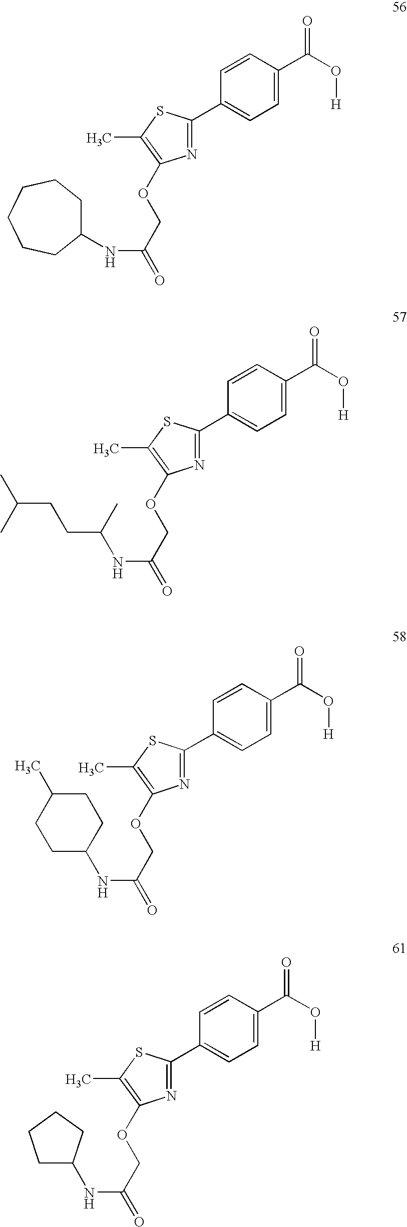

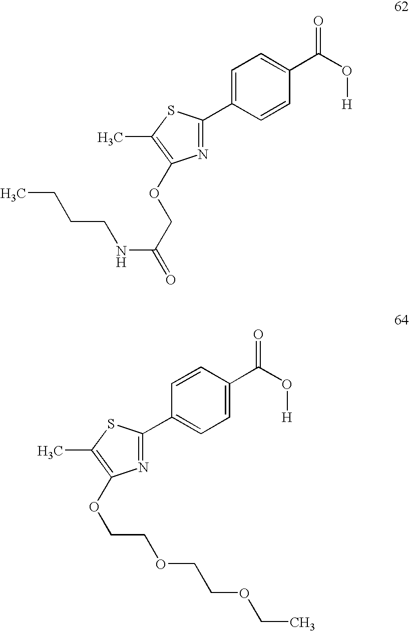

27. A method for the treatment of cancer or for alleviating cancer symptoms, comprising administering to a subject a therapeutically effective amount of at least one compound according to claim 1.

28. The method according to claim 27, further comprising coadministering a chemotherapeutic agent or radiation therapy.

29. The method according to claim 28, wherein the chemotherapeutic agent or radiation therapy is effective for the treatment of a cancer comprising a malignant tumor.

30. The method according to claim 29, wherein said cancer is selected from the group consisting of breast carcinoma and tumors in head, neck, lung, esophagus, mammary gland, pancreas, or cervix.

31. A method for the treatment of or for alleviating symptoms of a neurological disorder, comprising administering to a subject a therapeutically effective amount of at least one compound according claim 1.

32. The method according to claim 31, wherein said neurological disorder is selected from the group consisting of performance deficits in spatial learning and memory tasks, a disorder wherein cognition is altered, schizophrenia, and age-related memory deficit.

33. (canceled)

34. (canceled)

35. A method for the treatment of or for alleviating symptoms of a neurodegenerative disorder, comprising administering to a subject a therapeutically effective amount of at least one compound according to claim 1.

36. The method according to claim 35, wherein said neurodegenerative disorder is selected from the group consisting of Parkinson's disease, a motor neuron disease, a neurodegenerative disorder caused by a stroke, neurodegenerative disorder caused by nerve cell damage, neurodegenerative disorder caused by nerve cell damage due to spinal cord injury, a neurodegenerative disorder caused by nerve cell damage due to damage of cardiac muscles, a neurodegenerative disorder caused by islet cell damage in diabetes, a neurodegenerative disorder caused by multiple sclerosis, and Alzheimer's disease.

37. (canceled)

38. (canceled)

39. (canceled)

40. (canceled)

41. (canceled)

42. (canceled)

43. (canceled)

44. A method for the treatment of or for alleviating symptoms of a hyperproliferative or inflammatory disorder, comprising administering to a subject a therapeutically effective amount of at least one compound according to claim 1.

45. The method according to claim 44, wherein the inflammatory disorder is selected from the group consisting of a chronic inflammatory disorder, psoriasis, and rheumatoid arthritis.

46. (canceled)

47. The method according to claim 44, wherein said compound is given in combination with another drug effective to treat a hyperproliferative or inflammatory disorder.

48. The method according to claim 47, wherein said another drug is a TNF modulator, corticosteroid, or T-cell activation modulator.

49. The method according to claim 48, wherein said TNF modulator or T-cell activation modulator is selected from the group consisting of adalimumab, infliximab, etanercept, and efalizumab.

50. A method for treatment of or for alleviating symptoms of an eye disorder or an eye condition, comprising administering to a subject a therapeutically effective amount of at least one compound according to claim 1.

51. A method for treatment of or for alleviating symptoms of depression, comprising administering to a subject a therapeutically effective amount of at least one compound according to claim 1.

52. A method of identifying a compound which is an agonist, inverse agonist, or antagonist of one or more RARβ receptors, comprising:
contacting an RARβ receptor with at least one test compound according to claim 1; and determining any change in activity level of said one or more RARβ receptors so as to identify the test compound as an agonist, inverse agonist, or antagonist of one or more RARβ receptors.

53. A pharmaceutical composition comprising a compound according to claim 1 and at least one pharmaceutically acceptable adjuvant, excipient or carrier.

54. A compound according to claim 1 for use in the treatment of cancer or for alleviation of cancer symptoms.

55. The compound according to claim 54, wherein said treatment of cancer or alleviation of cancer symptoms is combined with chemotherapy or radiation therapy.

56. The compound according to claim 54, wherein the cancer comprises malignant tumors.

57. The compound according to claim 54, wherein said cancer is selected from the group consisting of breast carcinoma and tumors in head, neck, lung, esophagus, mammary gland, pancreas, or cervix.

58. A compound according to claim 1 for use in the treatment of or alleviating symptoms of a neurological disorder.

59. The compound according to claim 58, wherein said neurological disorder is selected from the group consisting of a neurological disorder is a disorder wherein cognition is altered, schizophrenia, and a performance deficit in spatial learning and memory tasks and/or an age-related memory deficit.

60. (canceled)

61. (canceled)

62. A compound according to claim 1 for use in the treatment of or alleviating symptoms of a neurodegenerative disorder.

63. The compound according to claim 62, wherein the neurodegenerative disorder is Parkinson's disease or Alzheimer's disease.

64. A compound according to claim 1 for use in the treatment of or alleviating symptoms of a hyperproliferative or inflammatory disorder.

65. The compound according to claim 64, wherein the inflammatory disorder is selected from the group consisting of a chronic inflammatory disorder, psoriasis, and rheumatoid arthritis.

66. (canceled)

67. A compound according to claim 1 for use in the treatment of or alleviating symptoms of an eye disorder or an eye condition.

68. A compound according to claim 1 for use in the treatment of or alleviating symptoms of depression.

69. A method for the preparation of a medicament for treating or alleviating symptoms of a disease or disorder associated with the RARβ receptor subtypes by using a compound, wherein the compound is a compound according to claim 1.

70. A method for modulating a RARβ receptor by using a compound, wherein the compound is a compound according to claim 1.
